(19) United States
(12) Patent Application Publication
(10) Pub. No.: US 2026/0137788 A1
(43) Pub. Date: May 21, 2026

(54) INFLAMMATION STIMULI RESPONSIVE PEPTIDE BRUSH POLYMERS

(71) Applicants: NORTHWESTERN UNIVERSITY, Evanston, IL (US); THE REGENTS OF THE UNIVERSITY OF CALIFORNIA, Oakland, CA (US)

(72) Inventors: Nathan C. GIANNESCHI, Evanston, IL (US); Matthew Peter THOMPSON, Evanston, IL (US); Spencer Tyler BURTON, Evanston, IL (US); Mary F. CASSIDY, Evanston, IL (US); Kendal P. CARROW, Evanston, IL (US); Karen L. CHRISTMAN, Oakland, CA (US); Joshua MESFIN, Oakland, CA (US); Holly SULLIVAN, Oakland, CA (US)

(73) Assignees: NORTHWESTERN UNIVERSITY, Evanston, IL (US); THE REGENTS OF THE UNIVERSITY OF CALIFORNIA, Oakland, CA (US)

(21) Appl. No.: 18/704,051

(22) PCT Filed: Nov. 11, 2022
(86) PCT No.: PCT/US2022/079733

§ 371 Date: Apr. 23, 2024

Related U.S. Application Data
(60) Provisional application No. 63/278,890, filed on Nov. 12, 2021.

(51) Int. Cl. A61K 47/58; A61K 47/62; A61P 29/00; B82Y 5/00

(52) U.S. Cl. CPC A61K 47/58; A61K 47/62; A61P 29/00; B82Y 5/00

(57) ABSTRACT

In an aspect, the invention provides compositions comprising brush block copolymers incorporating a combination of targeting agents and therapeutic agents. Compositions of the invention comprising brush block copolymers include high-density brush polymers for the targeted delivery of therapeutic agents. In an embodiment, brush block copolymers of the invention exhibit proteolysis-resistant characteristics and maintain their biological function during formulation and administration. The invention also includes methods of using compositions comprising brush block copolymers.

### CROSS-REFERENCE TO RELATED APPLICATIONS

This application claims the benefit of and priority to U.S. Provisional Patent Application No. 63/278,890, filed Nov. 12, 2021, which is hereby incorporated by reference in its entirety.

### STATEMENT REGARDING FEDERALLY SPONSORED RESEARCH OR DEVELOPMENT

This invention was made with government support under grant number HL139001 awarded by the National Institutes of Health. The government has certain rights in the invention.

### REFERENCE TO AN ELECTRONIC SEQUENCE LISTING

The content of the electronic sequence listing (339419_110-21_WO_ST1.xml; Size: 245,952 bytes; and Date of Creation: Nov. 11, 2022) is herein incorporated by reference in its entirety.

### BACKGROUND OF INVENTION

The use of protein and peptide therapeutics continues to increase dramatically for diverse clinical applications. However, inefficiencies in cellular uptake and rapid digestion by proteases are two problems that have limited the clinical adoption of peptide-based therapeutics. Accordingly, many peptide therapeutics are incompatible with systemic administration and, therefore, must be administered by injection at the site of action due to poor in vivo stability. This can result in poor patient compliance and, as such, many peptide therapies only are used clinically as salvage treatments.

One particular area of interest is in developing therapeutic delivery to treat myocardial infarctions. Myocardial infarction (MI), or a heart attack, affects 800,000 people a year in the United States. Atherosclerotic plaque buildup in the coronary arteries is one of the typical causes of MI due to a lack of blood flow to the heart, resulting in ischemic damage. The ischemic tissue damage leads to negative left ventricle (LV) remodeling, LV dilation, wall thinning, and finally heart failure.

Current traditional treatments for MI include stent administration, bypass grafting, drug therapies, and lifestyle changes. While methods such as coronary artery bypass grafting are effective for restoring blood flow, open-heart surgeries are higher risk. Catheter-based interventions, such as stent administration, are less invasive than surgical methods as they open occluded vessels. However, these treatments are only able to prevent future myocardial infarctions and are unable to prevent negative LV remodeling which eventually lead to heart failure and death. In addition, molecular-based therapies, such as anti-inflammatories, serve an important purpose towards treating the effects of improving cardiac function and mitigating negative LV remodeling. Yet, most anti-inflammatories target systemic pathways, which may exhibit a more detrimental patient outcome. As MI is a leading cause of heart failure, local delivery of these therapies would be beneficial to truly mitigate negative LV remodeling post-MI, improve patient outcomes, and prevent heart failure in MI patients.

Nanocarriers (NCs) present a potential non-invasive, targeted method of delivering therapeutic small molecules and peptides to the damaged myocardium post-MI. As seen in the acute MI disease phenotype, NCs have potential to deliver payloads directly to the target site of interest due the enhanced permeability and retention (EPR) effect, or otherwise known as “leaky vasculature”. Thus, NCs may be able to extravasate through the leaky vasculature in the infarct due to varying sizes of NCs ranging from 6 to 200 nm, allowing them to reach the damaged myocardium. Importantly, NCs have emerged as an attractive option for treating acute MI due to its minimally invasive nature. With NCs administered intravenously, there a possible an option to treat patients at acute timepoints. Finally, NCs are potentially compatible with a wide variety of drugs, leading to specific targeting of a therapeutic payload and a decreased drug dosage, which both are important to mitigating off-target effects due to systemic free-drug administration.

Enzyme responsive nanoparticles (NPs) are a type of NC that may aggregate and localize to areas with high matrix metalloproteinase (MMP) 2/9 activity. MMPs are enzymes that degrade the extracellular matrix in the heart, and most importantly are involved in the inflammatory cascade post-MI, seen as early as a few hours. Example NPs are made from peptide-polymer amphiphiles with a polynorbornene backbone and a MMP 2/9 cleavable peptide sequence, leading to NPs with hydrophilic and hydrophobic regions. After intravenous administration, these MMP-responsive NPs may utilize the EPR effect to reach the infarcted myocardium. In some embodiments, MMPs may cleave the MMP cleavable peptide present on the MMP-responsive NP, exposing the hydrophobic region of the NP, forming aggregates in the infarcts, and/or leading to retention of these micron-sized aggregates within the infarct.

MMP-responsive NP platform has potential for localization and retention in the injured myocardium. Considering the success of utilizing an inflammatory target seen in MI, inflammatory targets within the MI phenotype may be more intrinsic to negative LV remodeling for better targeting. LOX is an enzyme that is known to play a significant role in fibrotic diseases and disorders, such as MI, and is particularly known to contributing toward cardiac oxidative stress and the cardiac inflammatory response post-MI. Present in the extracellular matrix (ECM), LOX is an enzyme that is known to crosslink collagen and elastin, and has been shown to affect the metabolism of patients suffering from MI. Due to the crosslinking of collagen and elastin within the infarct which is present at later MI timepoints, LOX activity is suggested to increase over time. LOX activity leads to the creation of a fibrotic scar in the LV, to negative LV remodeling, and to less heart compliance over time. Utilizing the enzyme LOX as a mechanism to target the infarcted myocardium post-MI would be beneficial as it plays a significant role in negative LV remodeling and activity may increase over time. For example, as LOX may be a more chronic enzyme it may be relevant to determine whether LOX is expressed as early as MMPs are expressed in the MI phenotype for use with a responsive NC.

In addition, protein-like polymers (PLP) have promise for enzyme targeting in other tissues. PLPs exhibit hundreds of hours of half-life after systemic injection and have tunable properties, such as varying molecular weight, the ability to combine targeting groups with therapeutics, and have a more amorphous structure, leading to more protein-like behavior in circulation. Therefore, utilizing PLPs that more specifically target MI pathology may present a viable opportunity to treat MI while minimizing off target NC retention.

While LOX has been studied in chronic diseases and within MI, LOX is less well understood as a potential target of interest to target the infarcted heart in acute MI. Utilizing a LOX-responsive NC for clinical translation, therefore, has potential to open a wider time window for treating acute MI. Finally, conjugating a drug to either version of the NC may provide for improved retention over free-drug administration or drug encapsulation. Thus, the LOX-responsive NCs may be compatible with systemic injection in the presence of LOX in the infarct and allow for a less invasive method of administration to treat MI.

Another area of interest is in developing therapeutics targeting the protein-protein interaction between Nuclear factor (erythroid-derived 2)-like 2 (Nrf2) and Kelch-like ECH-Associating protein 1 (Keap1). The Keap1/Nrf2 interaction is important in a number of conditions including, for example, neurodegenerative diseases such as Alzheimer's and Parkinson's disease as well as heart and skin diseases among others. A therapeutic that successfully inhibits Keap1/Nrf2 binding can enhance the antioxidant and anti-inflammatory response to provide cytoprotective and neuroprotective effects for a number of disease states.

In this regard, Colarusso et al. (Bioorganic Med. Chem., 28; 1-12 (2020)) reports the optimization of linear and cyclic peptide inhibitors of Keap1/Nrf2 protein-protein interaction. More particularly, Colarusso et al. reports a library of linear peptides based on the Nrf2-binding motif SEQ ID NO: 1 (LDEETGEFL). However, the linear and cyclic peptide inhibitors of Keap1/Nrf2, disclosed by Colarusso et al. may suffer from substantial lack of cell permeability and were inactive. Thus, there remains a need for therapeutics targeting the protein-protein interaction between Nuclear factor (erythroid-derived 2)-like 2 (Nrf2) and Kelch-like ECH-Associating protein 1 (Keap1), which are cell permeable and enhance cellular Nrf2 activity.

Once the inefficiencies of cellular uptake are addressed, there remains the issue of digestion by proteases. Several approaches for producing peptides protected from proteolysis involve chemical modification of the amino acid sequence, which may be addressed via multiple rounds of structure-function studies to confirm that the activity of the peptide is not altered. Other approaches not using chemical modification of the amino acid sequence involve conjugation of the peptide to a pre-formed higher molecular weight structure, such as a polymer or nanomaterial. The downside of these approaches includes requiring additional conjugation and purification steps, as well as the formation of, and release from, the high molecular weight carrier.

Despite these challenges, there remains significant interest in developing improved delivery systems to enhance clinical applicability and overall efficacy for therapies involving NP therapeutic agents. Thus, there remains a need for NC delivery systems and methods for therapeutic agents, such as those targeting inflammation sites and the protein-protein interaction between Nrf2 and Keap1, which provide improved pharmacokinetic properties, administration routes and overall efficacies.

While the foregoing describes therapies directed to inflammation sites, MI reperfusion injuries, and protein-protein interactions between Nfr2 and Keap, it will be understood by one having skill in the art that the inventive delivery systems disclosed herein may be configured to deliver any of the therapeutic agents described herein.

### SUMMARY OF THE INVENTION

In an aspect, the invention provides a block copolymer characterized by a formula (FX1a):

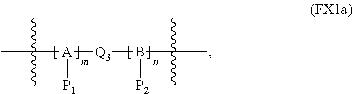

wherein each A is independently a first backbone monomer; each B is independently a second backbone monomer, wherein B is more hydrophilic than A; m is an integer selected from the range of 1 to 1000; n is an integer selected from the range of 1 to 1000; Q₃ is optionally present and is a linking group, optionally one or more polymer grafting groups; each P₁ is optionally present and is a therapeutic agent, optionally a therapeutic peptide, provided that at least one P₁ is present, and optionally a plurality of P₁ is present; and each P₂ is optionally present and is a targeting agent, optionally a targeting peptide, provided that at least one P₂ is present, and optionally a plurality of P₂ is present. In some embodiments, the block copolymer is characterized by a formula (FX1b):

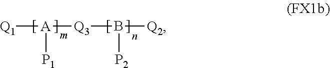

wherein Q₁ and Q₂ are each independently a polymer block terminating group.

In other aspects, the present invention comprises a pharmaceutical composition comprising any one of the block copolymers described herein and a pharmaceutically acceptable excipient.

The present invention further includes a method of treating or managing a condition of a subject comprising: administering to the subject a therapeutically effective amount of a block copolymer or a pharmaceutical composition comprising any one of the block copolymers described herein and a pharmaceutically acceptable excipient; wherein the administering results in the treating or managing of a condition of the subject.

Preferably in any embodiment of a block copolymer, a composition, a formulation, or a method of the invention disclosed herein, the block copolymers exhibit proteolysis-resistant characteristics and maintain their biological function during formulation and in vivo administration to a subject. In some embodiments, conjugation of the therapeutic agent to the first backbone monomer renders it more resistant to in vivo degradation by proteolytic enzymes as compared to a free therapeutic agent. Moreover, the higher molecular weight of the block copolymer, relative to its free therapeutic agent analogue, confers longer circulation time than the free therapeutic agent. As a result, the therapeutic polymers can be administered less frequently and in smaller doses than the free peptide therapeutics used in the clinic. Further, the enhanced stability and resistance to degradation of the present block copolymer therapeutic agents allows for more versatility with respect to administration route and conditions, including in injection at the site of action and systemic administration. Alternatively, or in addition to, the block copolymers of the invention may exhibit stronger binding affinity than the free therapeutic agent.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In addition, the drawings are not necessarily drawn to scale. In the drawings:

FIG. 1A depicts an exemplary structure of a block copolymer protein-like polymer (PLP) system.

FIG. 1B depicts a TEM image of aggregated PLPs following thermolysin treatment compared to drug-loaded nanoparticles (NPs) aggregation.

FIG. 1C depicts a cytocompatibility assessment of PLPs at various physiologically relevant concentrations when incubated with L929s.

FIG. 1D depicts a comparison of MMP-responsive PLP and MMP-responsive NP biodistribution imaged via LI-COR Odyssey at one-day post-injection.

FIG. 1E depicts two-way ANOVA results with MatLab quantification of scans from FIG. 1D. Šidik correction, ****p<0.0001, **p<0.01.

FIG. 2 depicts intracellular MMP-responsive PLP localization to the infarcted region of a left ventricle and colocalization with cardiomyocytes within the necrotic core of the infarct.

FIG. 3 depicts a comparison of PLP localization in vital organs based on PLP characteristics.

FIG. 4 depicts MMP-PLP retention in vital organs over a 28-day period post-injection.

FIG. 5 depicts MatLab quantification of LI-COR scans (not shown) of the heart and satellite organs comparing MMP-responsive PLP accumulation in vital organs for animals injected with DOX and animals injected with control (PBS).

FIG. 6A depicts the assembly of an exemplary LOX responsive PLP (LOX-PLP) through ring-opening metastasis polymerization (ROMP).

FIG. 6B depicts an exemplary structure of a LOX-PLP, the first magnified portion demonstrating a polynorborene backbone and the second magnified portion demonstrating an exemplary side chain.

FIG. 6C depicts potential modifications to a LOX-PLP formula for improvement, refinement, and/or adjustment of desirable properties.

FIG. 6D depicts functional domains of LOX enzyme.

FIG. 7A depicts an analytical HPLC absorbance spectrum for AAK monomer.

FIG. 7B depicts a Mass Spectra of AAK monomer.

FIG. 8A-8B depict a summary of reactions for the synthesis of AAK₂₀ (30 mM) PLPs from solid phase peptide synthesis and by reacting AAK monomer with Grubbs catalyst (III).

FIG. 8C depicts an NMR showing the rate of polymerization for the synthesis of AAK₂₀ PLP from AAK monomer.

FIG. 9A depicts the mass of the AAK₂₀ PLP on an SDS Page.

FIG. 9B-9D depict the physical characterizations of AAK₂₀ PLPs including dynamic light scattering (DLS) demonstrating of AAK₂₀ PLP diameter in PBS, diffusion ordered spectroscopy (DOSY) NMR demonstrating of AAK₂₀ PLP diffusion coefficient, and dry state TEM demonstrating of AAK₂₀ PLP globular assemblies.

FIG. 10A depicts LOX-PLP biodistribution during acute time points in heart and satellite organs by LI-COR.

FIG. 10B depicts a magnified portion of FIG. 10A, highlighting the LOX-PLP biodistribution in the heart.

FIG. 11A depicts LOX-PLP retention in infarcted myocardium through LI-COR intensity imaging.

FIG. 11B depicts quantification of LI-COR intensity images of FIG. 11A, comparing the intensity measurements of the LV with intensity measurements of the RV and septum.

FIG. 12A depicts a comparison of retentions of LOX and LOX-responsive PLP in an infarct with their retentions in a remote myocardium.

FIG. 12B depicts a confocal microscopy image showing LOX and LOX-responsive PLP colocalization with LOX and CD68 macrophages in an infarct.

FIG. 13A depicts a Volcano Plot comparing gene expression of tissue treated with MMPi responsive PLPs vs. saline.

FIG. 13B depicts an Enrichr graph displaying the enrichment of differentially expressed genes from samples treated with MMPi-responsive PLPs.

FIG. 13C depicts an Enhanced Volcano package in R to display differentially expressed genes between conditions.

FIG. 14A depicts qPCR results of LOX-responsive PLP, all statistical analysis was performed with 1-way-ANOVA. **: p<0.01; ***: p<0.001; ****: p<0.0001.

FIG. 14B depicts activity assay results of LOX-responsive PLP with LOX enzyme, all statistical analysis was performed with 1-way-ANOVA. **: p<0.01; ***: p<0.001; ****: p<0.0001.

FIG. 14C depicts a LOX-AAK₂₀ PLP Activity Assay in 3 healthy hearts as a control.

FIG. 14D depicts LOX-responsive PLP retention in an MI over 3 day period.

### STATEMENTS REGARDING CHEMICAL COMPOUNDS AND NOMENCLATURE

The following abbreviations are used herein: Keap1 refers to Kelch-like ECH-associated protein 1; Nrf2 refers to Nuclear factor-erythroid factor 2-related factor 2; MI refers to myocardial infarction; BBB refers to blood brain barrier; CNS refers to central nervous system; SPPS refers to solid phase peptide synthesis; ROMP refers to ring-opening metathesis polymerization; RAFT refers to reversible addition fragmentation chain transfer polymerization; DMF refers to dimethylformamide; TFA refers to trifluoroacetic acid; TIPS refers to triisopropyl silane; DTT refers to dithiothreitol; LJ refers to Lennard-Jones; RP-HPLC refers to reverse-phase high performance liquid chromatography; ESI-MS refers to electrospray ionization mass spectrometry; NMR refers to nuclear magnetic resonance spectrometry; MALDI-MS refers to matrix-assisted laser desorption/ionization mass spectrometry; SEC-MALS refers to size-exclusion chromatography coupled with multiangle light scattering; GPC refers to gel permeation chromatography; SDS-PAGE refers to sodium dodecyl sulfate-polyacrylamide gel electrophoresis; CD refers to circular dichroism; SAXS refers to Small-angle X-ray scattering; ARE refers to antioxidant response element; BSA refers to bovine serum albumin; tBHQ refers to tert-Butylhydroquinone; PLP refers to protein-like polymer; NP refers to nanoparticle; PDI refers to polydispersity index; MW refers to molecular weight; and DP refers to degree of polymerization.

Nanoparticles (NPs) are a type of nanocarrier (NC) capable of transporting small molecules throughout a subject, providing protection to small molecules from a surrounding environment, protecting the surrounding environment from biological activity of small molecules, and/or targeting delivery of small molecules to a specific site. NPs may be polymeric NPs, which generally have a size between the range of 1 to 1000 nm. NPs are generally categorized as nanospheres or nanocapsules. See Zielińska et al., Molecules, 25: 3731 (2020). As used herein, NPs configured to transport therapeutic agents are referred to as “drug-loaded NPs.”

In an embodiment, a peptide, a polymer, or a composition (e.g., formulation) of the invention is isolated or purified. In an embodiment, an isolated or purified peptide, polymer, or composition (e.g., formulation) is at least partially isolated or purified as would be understood in the art. In an embodiment, the peptide, polymer, or composition (e.g., formulation) of the invention has a chemical purity of at least 95%, optionally for some applications at least 99%, optionally for some applications at least 99.9%, optionally for some applications at least 99.99%, and optionally for some applications at least 99.999% pure. The invention includes isolated and purified compositions of any of the block copolymers (e.g., peptide brush copolymers) described herein including the brush block copolymers having one or more side chains comprising the peptide analogues, derivative, variants or fragments.

As used herein, the term “polymer” refers to a molecule composed of repeating structural units connected by covalent chemical bonds often characterized by a substantial number of repeating units (e.g., equal to or greater than 3 repeating units, optionally, in some embodiments equal to or greater than 5 repeating units, in some embodiments greater or equal to 10 repeating units) and an average molecular weight greater than or equal to 1 kDa, in some embodiments greater than or equal to 5 kDa, in some embodiments greater than or equal to 10 kDa and in some embodiments greater than or equal to 10 kDa. In some embodiments, the polymer is compatible with renal clearance and is characterized by an average molecular weight less than 50 kDa, optionally less than or equal to 35 kDa, and optionally less than or equal to 20 kDa. In some embodiments, the polymer is compatible with renal clearance and is characterized by an average molecular weight of 10 kDa to less than 50 kDa and optionally of 15 kDa to 35 kDa and optionally of 20 kDa to 35 kDa.

Polymers are commonly the polymerization product of one or more monomer precursors. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits (e.g., 3 or more monomer subunits, 4 or more monomer subunits, 5 or more monomer subunits, 6 or more monomer subunits), and include random, block, brush, brush block, alternating, segmented, grafted, tapered and other architectures. In some embodiments, copolymers of the invention comprise from 2 different monomer subunits. Useful polymers include organic polymers that may be in amorphous, semi-amorphous, crystalline or semi-crystalline states. Cross linked polymers having linked monomer chains are useful for some applications, for example linked by one or more disulfide linkages. The invention provides polymers comprising therapeutic agents, such as brush polymers having at least a portion of the repeating units comprising polymer side chains such as peptide side chains. The invention also provides polymers comprising targeting agents, such as block copolymers having at least a portion of the repeating units comprising polymer side chains such as targeting agent side chains.

As used herein, the term “polymer segment” (e.g., first polymer segment, second polymer segment, etc.) refers to a section (e.g., portion) of the polymer comprising a particular monomer or arrangement of monomers. A polymer segment can be a homopolymer or a copolymer. In embodiments where a polymer segment is a copolymer, the copolymer can exist in any suitable arrangement of monomers (e.g., random, block, brush, brush block, alternating, segmented, grafted, tapered, statistical and other architectures). In some embodiments, the polymer segments are homopolymers, random copolymers, statistical copolymers, or block copolymers. Any polymer (e.g., brush polymer) described herein can have a single polymer segment or multiple polymer segments. In embodiments where the polymer has multiple polymer segments, the polymer segments can exist in any suitable arrangement (random, block, brush, brush block, alternating, segmented, grafted, tapered, statistical, and other architectures).

As used herein, the term “backbone monomer” (e.g., first backbone monomer and second backbone monomer) refers to a section of a polymer backbone comprising a particular monomer. The present invention preferably comprises a block copolymer wherein the second backbone monomer is more hydrophilic than the first backbone monomer.

An “oligomer” refers to a molecule composed of repeating structural units connected by covalent chemical bonds often characterized by a number of repeating units less than that of a polymer (e.g., equal to or less than 3 repeating units) and a lower molecular weights (e.g., less than or equal to 1,000 Da) than polymers. Oligomers may be the polymerization product of one or more monomer precursors.

A “peptide” or “oligopeptide” herein are used interchangeably and refer to a polymer of repeating structural units connected by a peptide bond. Typically, the repeating structural units of the peptide are amino acids including naturally occurring amino acids, non-naturally occurring amino acids, analogues of amino acids or any combination of these. The number of repeating structural units of a peptide, as understood in the art, are typically less than a “protein”, and thus the peptide often has a lower molecular weight than a protein. In some embodiments, a peptide has a chain length of 3 to 150 amino acids, optionally 3 to 100 amino acids, optionally 5 to 50 amino acids, and optionally 5 to 20 amino acids.

As used herein, the term “targeting agent” refers to an agent that directs transport of a polymer to a specific region of a subject. In embodiments, the targeting agent directs transport to a disease site of a subject, an inflammation site of a subject, a tumor of a subject, a tissue of a subject, an organ or an organelle of a subject, a cell of a subject, an intracellular receptor of a subject, an extracellular receptor of a subject, a transmembrane receptor of a subject, an enzyme of a subject, a protein-protein interaction of a subject, or any combination thereof. In embodiments, the targeting agent facilitates localization and/or aggregation of the polymer at the target site. In some embodiments, the targeting agent is a targeting peptide. In some embodiments, a targeting agent comprises a targeting peptide having a chain length of 3 to 150 amino acids, optionally of 3 to 100 amino acids, optionally 5 to 50 amino acids, optionally 5 to 20 amino acids. The targeting peptide may be a naturally-occurring peptide, a synthetic peptide, or a purified recombinant peptide. In some embodiments, the targeting peptide is characterized by an average molecular weight less than or equal to 40 kDa, optionally less than or equal to 30 kDa, optionally less than or equal to 20 kDa, optionally less than or equal to 10 kDa, and optionally less than or equal to 5 kDa. In some embodiments, the targeting peptide is characterized by an average molecular weight of 0.5 kDa to 20 kDa, optionally of 0.5 kDa to 10 kDa and optionally of 1 kDa to 5 kDa.

A “LOX peptide” herein refers to a targeting agent configured to target a lysyl oxidase (LOX) enzyme. In some embodiments, the LOX peptide has an amino acid sequence corresponding to a substrate of a LOX enzyme. In embodiments, the substrate may be an activator or an inhibitor of the LOX enzyme. In some aspects, the LOX peptide facilitates crosslinking of the block copolymer with tissue of the target site by the LOX enzyme.

A “MMP peptide” herein refers to a targeting agent configured to target a matrix metalloproteinase (MMP) enzyme (also known as matrix metallopeptidase or matrixin). In some embodiments, the MMP peptide has an amino acid sequence corresponding to a substrate of a MMP enzyme. In embodiments, the substrate may be an activator or an inhibitor of the MMP enzyme.

As used herein, the term “responsive” refers to an agent or a peptide wherein at least a portion of its formulation is capable of interacting with at least a portion of a specific molecule. For example, a responsive peptide may include an amino acid sequence corresponding to a cut-site for a specific enzyme.

As used herein, the term “nonresponsive” refers to an agent or a peptide having a formulation that is not known to interact with a specific molecule.

The term “therapeutic agent” as used herein refers to a class of agents capable of treating or managing a disease, illness, or other condition of a subject. In some embodiments, the therapeutic agent is a pharmaceutical or biological agent or component or fragment thereof. In an embodiment, the therapeutic agent is a therapeutic peptide. In embodiments, the therapeutic agent may be a therapeutic peptide having a chain length of 3 to 150 amino acids, optionally of 3 to 100 amino acids, optionally 5 to 50 amino acids and optionally 5 to 20 amino acids. The therapeutic peptide may be a naturally-occurring peptide, a synthetic peptide, or a purified recombinant peptide. In other embodiments, the therapeutic agent may be a small molecule therapeutic. In examples, the small molecule therapeutic comprises a low molecular weight organic compound having a size less than or equal to 20 nm, optionally less than or equal to 15 nm, optionally less than or equal to 10 nm. In some embodiments, the therapeutic peptide is characterized by an average molecular weight less than or equal to 40 kDa, optionally less than or equal to 30 kDa, optionally less than or equal to 20 kDa, optionally less than or equal to 10 kDa, and optionally less than or equal to 5 kDa. In some embodiments, the therapeutic peptide is characterized by an average molecular weight of 0.5 kDa to 20 kDa, optionally of 0.5 kDa to 10 kDa and optionally of 1 kDa to 5 kDa.

“Block copolymers” are a type of copolymer comprising blocks or spatially segregated domains, wherein different domains comprise different polymerized monomers, for example, including at least two chemically distinguishable blocks. Block copolymers may further comprise one or more other structural domains, such as hydrophobic groups, hydrophilic groups, etc. In a block copolymer, adjacent blocks are constitutionally different, i.e., adjacent blocks comprise constitutional units derived from different species of monomer or from the same species of monomer but with a different composition or sequence distribution of constitutional units. Different blocks (or domains) of a block copolymer may reside on different ends or the interior of a polymer (e.g., [A][B]), or may be provided in a selected sequence ([A][B][A][B]). “Diblock copolymer” refers to block copolymer having two different polymer blocks. “Triblock copolymer” refers to a block copolymer having three different polymer blocks, including compositions in which two non-adjacent blocks are the same or similar. “Pentablock” copolymer refers to a copolymer having five different polymer including compositions in which two or more non-adjacent blocks are the same or similar.

“Random copolymers” are a type of copolymer comprising spatially randomized units, wherein at least two chemically distinguishable polymerized monomers are randomly distributed throughout the polymer.

As used herein, “Protein-like Polymers (PLPs)” are polymers capable of targeting and delivering therapeutic agents to a specific site within a subject with enhanced circulation time and without rapid proteolytic peptide degradation upon systemic administration. PLPs comprise a single, dissolved, dispersed polymer chain with tunable molecular weight, and protein-like qualities. In embodiments, PLPs are capable of delivering therapeutic agents, such as anti-inflammatory therapeutics, with high efficacy, without non-specific accumulation in satellite organs.

“Polymer backbone group” refers to groups that are covalently linked to make up a backbone of a polymer, such as a block copolymer or a random copolymer. Polymer backbone groups may be linked to side chain groups, such as polymer side chain groups. Some polymer backbone groups useful in the present compositions are derived from polymerization of a monomer selected from the group consisting of a substituted or unsubstituted norbornene, olefin, cyclic olefin, norbornene anhydride, cyclooctene, cyclopentadiene, styrene, acrylamide, and acrylate. Some polymer backbone groups useful in the present compositions are obtained from a ring opening metathesis polymerization (ROMP) reaction. Polymer backbones may terminate in a range of backbone terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen, C₁-C₁₀ alkyl or C₅-C₁₀ aryl.

“Polymer side chain group” refers to a group covalently linked (directly or indirectly) to a polymer backbone group that comprises a polymer side chain, optionally imparting steric properties to the polymer. In an embodiment, for example, a polymer side chain group is characterized by a plurality of repeating units having the same, or similar, chemical composition, such as a plurality of amino acids. In preferred embodiments, a polymer side chain group is characterized by either a therapeutic agent or a targeting agent. A polymer side chain group may be directly or indirectly linked to the polymer backbone groups. In some embodiments, polymer side chain groups provide steric bulk and/or interactions that result in an extended polymer backbone and/or a rigid polymer backbone. Some polymer side chain groups useful in the present compositions include unsubstituted or substituted peptide groups. Some polymer side chain groups useful in the present compositions comprise repeating units obtained via anionic polymerization, cationic polymerization, free radical polymerization, group transfer polymerization, or ring-opening polymerization. A polymer side chain may terminate in a wide range of polymer side chain terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen or C₁-C₅ alkyl.

As used herein, the term “degree of polymerization” refers to the average number of monomer units per polymer chain. For example, for certain polymers described herein, comprising A, B, and/or Q₃ monomer units, the degree of polymerization would be represented by the sum total of A, B, and Q₃ monomer units. Since the degree of polymerization can vary from polymer to polymer, the degree of polymerization is generally represented by an average.

In embodiments, Q₃ is a linking group, and optionally a linking group comprising a polymer grafting group. In some embodiments, Q₃ comprises one or more first backbone monomers, second backbone monomers, additional backbone monomers or any derivatives, substituents, portions or fragments thereof, optionally functionalized by one or more additional substitutents, such as peptide substituents or substituents derived from small molecules. In some embodiments, Q₃ comprises a linking group selected from the group consisting of single bond, —O—, C₁-C₂₀ alkylene, C₂-C₂₀ heteroalkylene, C₃-C₂₀ cycloalkylene, C₃-C₂₀ arylene, C₃-C₂₀ heteroarylene, C₂-C₂₀ alkenylene, C₂-C₂₀ cycloalkenylene, C₁-C₂₀ alkoxy, C₁-C₁₀ acyl and combinations thereof. In some embodiment, Q₃ comprises a dye, chromaphore, flourophor or other probe, visualization or imaging agent.

As used herein, the term “brush polymer” refers to a polymer comprising repeating units each independently comprising a polymer backbone group covalently linked to at least one polymer side chain group. A brush polymer may be characterized by “brush density” which refers to the percentage of the repeating units comprising polymer side chain groups, wherein the polymer side chain groups are independently a therapeutic agent or a targeting agent. Brush polymers of certain aspects have a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects have a brush density selected from the range 50% to 100%, optionally some embodiments a density selected from the range of 75% to 100%, or optionally for some embodiments a density selected from the range of 90% to 100%. Brush polymers of certain aspects have a “high brush density” selected from the range 90% to 100%, optionally some embodiments a density selected from the range of 95% to 100%, or optionally for some embodiments a density selected from the range of 99% to 100%.

As used herein, the term “P₁ density” refers to the percentage of first backbone monomer units in the polymer chain which have a therapeutic agent covalently linked thereto. The percentage is based on the overall sum of first backbone monomer units in the polymer chain. For example, for certain polymers described herein, each P₁ is the polymer side chain comprising the therapeutic agent, each P₀ indicates the absence of the therapeutic agent polymer side chain. Thus, the P₁ density, or percentage of first backbone monomer units comprising the therapeutic agent, may be represented by the formula:

$$\frac{{P}_{1}}{{P}_{1} + {P}_{0}} \times 100 ,$$

where each variable refers to the number of monomer units of that type in the polymer chain. Polymers of certain aspects have a P₁ density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Polymers of certain aspects have a P₁ density selected from the range 50% to 100%, optionally some embodiments a density selected from the range of 75% to 100%, optionally for some embodiments a density selected from the range of 90% to 100%, optionally for some embodiments a density selected from the range of 95% to 100%, or optionally for some embodiments a density selected from the range of 99% to 100%. In some embodiments, the brush density is equal to the P₁ density. In embodiments wherein the P₁ density is less than 100%, at least a portion of the monomers will comprise the backbone unit, or a derivative, substituent, portion or fragment thereof, without P₁ present. For example, when the P₁ density is less than 100%, monomers without P₁ present can be any monomer described herein wherein P₁ has been replaced by any suitable substituent (e.g., hydrogen, OH, or NH₂) such that the valency on the monomer is filled.

As used herein, the term “P₂ density” refers to the percentage of second backbone monomer units in the polymer chain which have a targeting agent covalently linked thereto. The percentage is based on the overall sum of second backbone monomer units in the polymer chain. For example, for certain polymers described herein, each P₂ is the polymer side chain comprising the targeting agent, each P₀ indicates the absence of the targeting agent polymer side chain. Thus, the P₂ density, or percentage of second backbone monomer units comprising the targeting agent, may be represented by the formula:

$$\frac{{P}_{2}}{{P}_{2} + {P}_{0}} \times 100 ,$$

where each variable refers to the number of monomer units of that type in the polymer chain. Polymers of certain aspects have a P₂ density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Polymers of certain aspects have a P₂ density selected from the range 50% to 100%, optionally some embodiments a density selected from the range of 75% to 100%, optionally for some embodiments a density selected from the range of 90% to 100%, optionally for some embodiments a density selected from the range of 95% to 100%, or optionally for some embodiments a density selected from the range of 99% to 100%. In some embodiments, the brush density is equal to the P₂ density. In embodiments wherein the P₂ density is less than 100%, at least a portion of the monomers will comprise the backbone unit, or a derivative, substituent, portion or fragment thereof, without P₂ present. For example, when the P₂ density is less than 100%, monomers without P₂ present can be any monomer described herein wherein P₂ has been replaced by any suitable substituent (e.g., hydrogen, OH, or NH₂) such that the valency on the monomer is filled.

In an aspect, the polymer side chain groups can have any suitable spacing on the polymer backbone. Typically, the space between adjacent polymer side chain groups is from 3 angstroms to 30 angstroms, and optionally 5 to 20 angstroms and optionally 5 to 10 angstroms. By way of illustration, in certain embodiments having a brush density of 100%, the polymer side chain groups typically are spaced 6±5 angstroms apart on the polymer backbone. In some embodiments the brush polymer has a high a brush density (e.g., greater than 70%), wherein the polymer side chain groups are spaced 5 to 20 angstroms apart on the polymer backbone.

The term “sequence homology” or “sequence identity” means the proportion of amino acid matches between two amino acid sequences. When sequence homology is expressed as a percentage, e.g., 50%, the percentage denotes the fraction of matches over the length of sequence that is compared to some other sequence. Gaps (in either of the two sequences) are permitted to maximize matching; for example, wherein gap lengths of 5 amino acids or less, optionally 3 amino acids or less, are usually used. In other words, a sequence having 75% or greater sequence identity of SEQ ID NO: 1 (LDEETGEFL) or SEQ ID NO: 2 (LDPETGEFL) can indicate that the foregoing sequences can have one or two point mutations (i.e., amino acid change), one or two amino acid deletions, one or two amino acid additions, one point mutation and one amino acid deletion, or one point mutation and one amino acid addition. Similarly, a sequence having 85% or greater sequence identity of SEQ ID NO: 1 (LDEETGEFL) or SEQ ID NO: 2 (LDPETGEFL) indicates that the foregoing sequences can have one point mutation (i.e., amino acid change), one amino acid deletion, or one amino acid addition.

The term “fragment” refers to a portion, but not all of, a composition or material, such as a peptide composition or material. In an embodiment, a fragment of a peptide refers to 50% or more of the sequence of amino acids, optionally 70% or more of the sequence of amino acids and optionally 90% or more of the sequence of amino acids.

As used herein, the phrase “charge modulating domain” refers to one or more amino acids added to the peptide sequences described herein to modulate the charge of the peptide. For example, the charge modulating domain can be a TAT sequence, a glycine-serine domain, a cationic residue domain, or a combination thereof, or optionally a glycine-serine domain, a cationic residue domain, or a combination thereof. In certain embodiments, the charge modulating domain has from 2 to 7 amino acid residues. The 2 to 7 amino acids can be added in a single block containing from 2 to 7 amino acid residues or more than one block containing from 1 to 6 amino acid residues. In preferred embodiments, the charge modulating domain is a cationic residue domain having from 2 to 7 amino acid residues selected from lysine, arginine, histidine, or a combination thereof. Generally, the charge modulating domain modulates the charge of the peptide to have a net positive charge. Without wishing to be bound by any particular theory, it is believed that the net positive charge increases the cellular uptake of the peptide or polymer comprising the peptide. The overall charge of the peptide or copolymer comprising the peptide can be determined by any suitable means. For example, the overall charge can be determined by (i) structural analysis of the functional residues on the peptide sequence and their respective pKa, (ii) physical characterization by measuring the zeta potential, and/or (iii) by virtue of the material moving towards a negative pole in an electrophoresis polymer gel. In certain embodiments, the overall charge of the peptide or copolymer comprising the peptide is determined by measuring the zeta potential.

“Polymer blend” refers to a mixture comprising at least one polymer, such as a brush polymer, e.g., brush block copolymer or brush random copolymer, and at least one additional component, and optionally more than one additional component. In some embodiments, for example, a polymer blend of the invention comprises a first brush copolymer and one or more addition brush polymers having a composition different than the first brush copolymer. In some embodiments, for example, a polymer blend of the invention further comprises one or more additional brush block copolymers, brush random copolymers, homopolymers, copolymers, block copolymers, random copolymers, brush block copolymers, oligomers, solvent, small molecules (e.g., molecular weight less than 500 Da, optionally less than 100 Da), or any combination of these. Polymer blends useful for some applications comprise a first brush polymer, and one or more additional components comprising polymers, block copolymers, brush polymers, linear block copolymers, random copolymers, homopolymers, or any combinations of these. Polymer blends of the invention include mixture of two, three, four, five and more polymer components.

As used herein, the term “compound” can be used to refer to any of the agents, peptides, or polymers described herein. Alternatively, or additionally, the term compound can refer to any of the synthetic precursors, reagents, additives, excipients, etc. used in preparation of or formulation with the agents, peptides, or polymers described herein.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

As used herein, the term “substituted” refers to a compound wherein a hydrogen is replaced by another functional group.

Unless otherwise specified, the term “average molecular weight,” refers to number average molecular weight. Number average molecular weight is the defined as the total weight of a sample volume divided by the number of molecules within the sample. As is customary and well known in the art, peak average molecular weight and weight average molecular weight may also be used to characterize the molecular weight of the distribution of polymers within a sample.

As is customary and well known in the art, hydrogen atoms in formulas (FX1a)-(FX1e3) are not always explicitly shown, for example, hydrogen atoms bonded to the carbon atoms of aromatic, heteroaromatic, and alicyclic rings are not always explicitly shown in formulas ((FX1a)-(FX1e3)). The structures provided herein, for example in the context of the description of formulas (FX1a)-(FX1e3) and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g., L₁-L₂).

As used herein, the terms “heteroalkylene” and “heteroalkylene group” are used synonymously and refer to a divalent group derived from an alkyl group as defined herein, which further comprises one or more heteroatoms (e.g., polyethylene glycol, polypropylene glycol, or combinations thereof). Heteroalkylene groups in some compounds function as linking and/or spacer groups. Compounds of the invention may have substituted and/or unsubstituted C₁-C₂₀ heteroalkylene, C₁-C₁₀ heteroalkylene and C₁-C₅ heteroalkylene groups, for example, as one or more linking groups (e.g., L₁-L₂).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g., L₁-L₂).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g., L₁-L₂).

As used herein, the terms “heteroarylene” and “heteroarylene group” are used synonymously and refer to a divalent group derived from a heteroaryl group as defined herein. The invention includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g., L₁-L₂).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g., L₁-L₂).

As used herein, the terms “cycloalkenylene” and “cycloalkenylene group” are used synonymously and refer to a divalent group derived from a cycloalkenyl group as defined herein. The invention includes compounds having one or more cycloalkenylene groups. Cycloalkenylene groups in some compounds function as linking and/or spacer groups. Compounds of the invention include substituted and/or unsubstituted C₃-C₂₀ cycloalkenylene, C₃-C₁₀ cycloalkenylene and C₃-C₅ cycloalkenylene groups, for example, as one or more linking groups (e.g., L₁-L₂).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g., L₁-L₂).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy)n-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides are comprised of two or more amino acids connected via peptide bonds.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—. Compositions of some embodiments of the invention comprise alkyl groups as terminating groups, such as polymer backbone terminating groups and/or polymer side chain terminating groups.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including —CN;

—COOR where R is a hydrogen or an alkyl group or an aryl group and more specifically where R is a methyl, ethyl, propyl, butyl, or phenyl group all of which groups are optionally substituted;

—COR where R is a hydrogen or an alkyl group or an aryl group and more specifically where R is a methyl, ethyl, propyl, butyl, or phenyl group all of which groups are optionally substituted;

—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;

—SR, where R is hydrogen or an alkyl group or an aryl group and more specifically where R is hydrogen, methyl, ethyl, propyl, butyl, or a phenyl group, which are optionally substituted;

—SO₂R, or —SOR where R is an alkyl group or an aryl group and more specifically where R is a methyl, ethyl, propyl, butyl, or phenyl group, all of which are optionally substituted;

—OCOOR where R is an alkyl group or an aryl group;

—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;

—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, which is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or D- or L-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. Isomers include structural isomers and stereoisomers such as enantiomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to a subject, such as a patient in need of treatment; slowing in the rate of degeneration or decline; making the final point of degeneration less debilitating; improving a subject's physical or mental well-being. The treatment or amelioration of symptoms can be based on objective or subjective parameters; including the results of a physical examination, neuropsychiatric exams, and/or a psychiatric evaluation.

An “effective amount” is an amount sufficient to accomplish a stated purpose (e.g., achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g., transcription factor) relative to the absence of the antagonist. An “activity increasing amount,” as used herein, refers to an amount of agonist (activator) required to increase the activity of an enzyme or protein (e.g., transcription factor) relative to the absence of the agonist. A “function disrupting amount,” as used herein, refers to the amount of antagonist (inhibitor) required to disrupt the function of an enzyme or protein (e.g., transcription factor) relative to the absence of the antagonist. A “function increasing amount,” as used herein, refers to the amount of agonist (activator) required to increase the function of an enzyme or protein (e.g., transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g., agonist) interaction means positively affecting (e.g., increasing) the activity or function of the protein.

The term “modulator” refers to a composition that increases or decreases the level of a target molecule or the function of a target molecule.

“Patient” “subject” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a mammal. In some embodiments, a patient is a mouse. In some embodiments, a patient is an experimental animal. In some embodiments, a patient is a rat. In some embodiments, a patient is a test animal.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent or chemotherapeutic). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995); as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm.46:1576-1587, 1989).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der waal's bond(s)/interactions, hydrogen bond(s), polar bond(s), or combinations or mixtures thereof).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

### DETAILED DESCRIPTION OF THE INVENTION

In the following description, numerous specific details of the compounds, compositions components and methods of the present invention are set forth in order to provide a thorough explanation of the precise nature of the invention. It will be apparent, however, to those of skill in the art that the invention can be practiced without these specific details.

In an aspect, the invention provides a block copolymer that is characterized by a formula (FX1a):

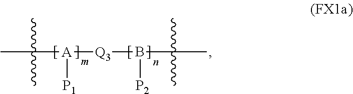

wherein each A is independently a first backbone monomer; each B is independently a second backbone monomer, wherein B is more hydrophilic than A; m is an integer selected from the range of 1 to 1000; n is an integer selected from the range of 1 to 1000; Q₃ is optionally present and is a linking group, optionally a polymer grafting group; each P₁ is optionally present and is a therapeutic agent, optionally a therapeutic peptide provided that at least one P₁ is present, and optionally a plurality of P₁ is present; and each P₂ is optionally present and is a targeting agent, optionally a targeting peptide provided that at least one P₂ is present, and optionally a plurality of P₂ is present.

In embodiments, the second backbone monomer being more hydrophilic than the first backbone monomer contributes to enhanced biological activities, such as higher binding affinities to targets, increased cellular penetration, improved circulation half-life in a subject, and non-specific enzyme resistance. In certain aspects, the relative hydrophilicity of the backbone monomers contributes to stability in a subject for at least 3 days, optionally at least 7 days. In certain embodiments, the present invention is stable in a subject for at least 14 days, optionally 30 days.

In aspects of the invention, at least one of the first backbone monomers will have a therapeutic agent attached. In embodiments where at least one of the first backbone monomers does not have a therapeutic agent attached, at least one of said first backbone monomers may have a spacer group attached. In further aspects, the spacer group may comprise an alkylene group, a heteroalkylene group, a cycloalkyl group, an arylene group, a heteroarylene group, an alkenylene group, a cycloalkenylene group, or an alkynylene group.

In aspects of the invention, at least one of the second backbone monomers will have a targeting agent attached. In embodiments where at least one of the second backbone monomers does not have a targeting agent attached, at least one of said second backbone monomers may have a spacer group attached. In further aspects, the spacer group may comprise an alkylene group, a heteroalkylene group, a cycloalkyl group, an arylene group, a heteroarylene group, an alkenylene group, a cycloalkenylene group, or an alkynylene group.

Provided in further aspects of the invention, the block copolymer is characterized by a formula (FX1b):

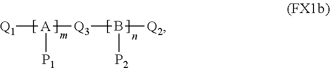

wherein Q₁ and Q₂ are each independently a polymer block terminating group. Each of Q₁ and Q₂ may independently comprise hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen, C₁-C₁₀ alkyl or C₅-C₁₀ aryl.

The inventive block copolymer may comprise a first backbone monomer characterized by a formula (FX1c):

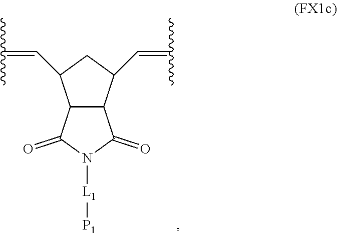

wherein L₁ is optionally present and is a first linking group.

In some embodiments, the block copolymer comprises a second backbone monomer characterized by a formula (FX1d) or (FX1e):

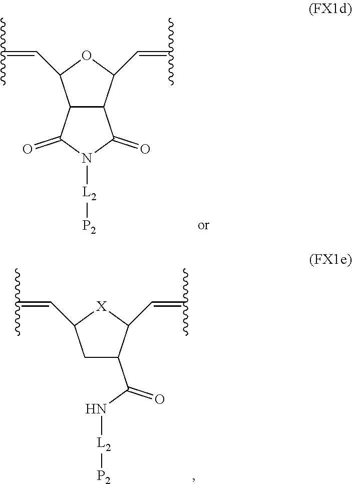

wherein L2 is optionally present and is a second linking group, and X is CH₂ or O. In other embodiments, the second backbone monomer is characterized by a formula (FX1e):

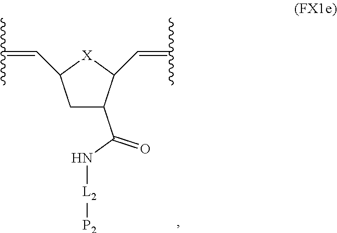

wherein X is CH₂.

In aspects, each of the linking groups (i.e., L₁ and L2) may independently comprise an alkylene group, a heteroalkylene group, a cycloalkyl group, an arylene group, a heteroarylene group, an alkenylene group, a cycloalkenylene group, an alkynylene group, an alkoxy group, an acyl group, a triazole group, a diazole group, a pyrazole group, and combinations thereof. In aspects, the inventive block copolymer may have substituted and/or unsubstituted C₁-C₂₀ alkylene, C₁-C₁₀ alkylene and C₁-C₅ alkylene groups, for example, as one or more linking groups. In aspects, the inventive block copolymer may have substituted and/or unsubstituted C₁-C₂₀ heteroalkylene, C₁-C₁₀ heteroalkylene and C₁-C₅ heteroalkylene groups, for example, as one or more linking groups. In aspects, the inventive block copolymer may include substituted and/or unsubstituted C₃-C₂₀ cycloalkylene, C₃-C₁₀ cycloalkylene and C₃-C₅ cycloalkylene groups, for example, as one or more linking groups. In aspects, the inventive block copolymer may have substituted and/or unsubstituted C₃-C₃₀ arylene, C₃-C₂₀ arylene, C₃-C₁₀ arylene and C₁-C₅ arylene groups, for example, as one or more linking groups. In aspects, the inventive block copolymer may include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups. In aspects, the inventive block copolymer may include substituted and/or unsubstituted C₂-C₂₀ alkenylene, C₂-C₁₀ alkenylene and C₂-C₅ alkenylene groups, for example, as one or more linking groups. In aspects, the inventive block copolymer may include substituted and/or unsubstituted C₃-C₂₀ cycloalkenylene, C₃-C₁₀ cycloalkenylene and C₃-C₅ cycloalkenylene groups, for example, as one or more linking groups. In aspects, the inventive block copolymer may include substituted and/or unsubstituted C₂-C₂₀ alkynylene, C₂-C₁₀ alkynylene and C₂-C₅ alkynylene groups, for example, as one or more linking groups. In certain embodiments, each of L₁ and L2 is independently selected from a single bond, O—, C₁-C₁₀ alkyl, C₂-C₁₀ alkylene, C₁-C₁₀ heteroalkylene, C₃-C₁₀ arylene, C₁-C₁₀ alkoxy, C₁-C₁₀ acyl and combinations thereof.

In some embodiments, each of L₁ and L2 is independently selected from C₁-C₂₀ alkylene and C₁-C₂₀ heteroalkylene, optionally terminated in a carbonyl, an amine, or an amide. Thus, in some embodiments, each of L₁ and L2 is independently selected from C₁-C₂₀ alkylene and C₁-C₂₀ heteroalkylene, which C₁-C₂₀ alkylene and C₁-C₂₀ heteroalkylene further comprises the necessary atoms (e.g., a carbonyl or an amine) to form an amide bond with the peptide. In other words, L₁ and L2 can be alkylene or heteroalkylene groups, which are bound to the peptide via an amide bond. For example, L₁ and L2 can be independently selected from —(CH₂)ₙNR—, —(CH₂)ₙC(O)NR—, —(CH₂)ₙNRC(O)—, —(CH₂)ₙC(O)— and —(CH₂)ₙ—, wherein n is an integer from 1 to 20 and R is hydrogen or a C₁-C₅ alkyl. In some embodiments, L₁ and L₂ can be independently selected from —(CH₂)ₙNR—, —(CH₂)ₙC(O)NR—, —(CH₂)ₙNRC(O)—, —(CH₂)ₙC(O)— and —(CH₂)ₙ—, wherein n is an integer from 1 to 10 and R is hydrogen.

In some embodiments, each of L₁ and/or L₂ further comprises an enzymatically degradable linker, and wherein at least a portion of each of P₁ and/or P₂ is independently linked to the enzymatically degradable linker. The enzymatically degradable linker may comprise a MMP cleavage sequence, a cathepsin B cleavage sequence, an ester bond, a reductive sensitive bond-disulfide bond, a pH sensitive bond-imine bond, or any combination thereof. In embodiments, the enzymatically degradable linker is a carbamate.

In some embodiments, the inventive block copolymer comprises a first backbone monomer characterized by a formula (FX1c1), (FX1c2), or (FX1c3):

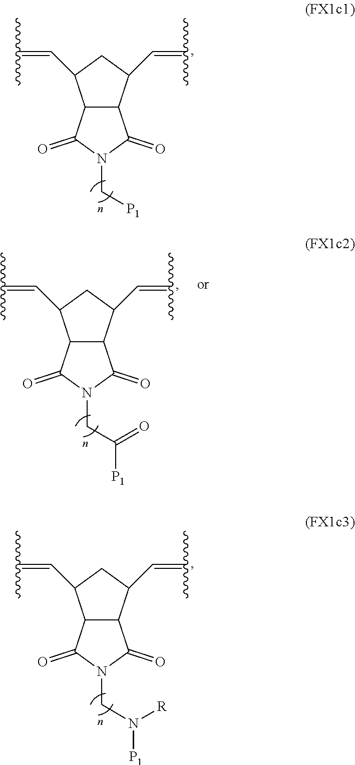

wherein n is an integer from 1 to 20 (e.g., 1, 2, 3, 4, 5, 6, 7, 8, 9, 10, 11, 12, 13, 14, 15, 16, 17, 18, 19, or 20) and R is hydrogen or a C₁-C₅ alkyl. In preferred embodiments, R is hydrogen.

In some embodiments, the inventive block copolymer comprises a second backbone monomer characterized by a formula (FX1d1), (FX1d2), (FX1d3), (FX1e1), (FX1e2), or (FX1e3):

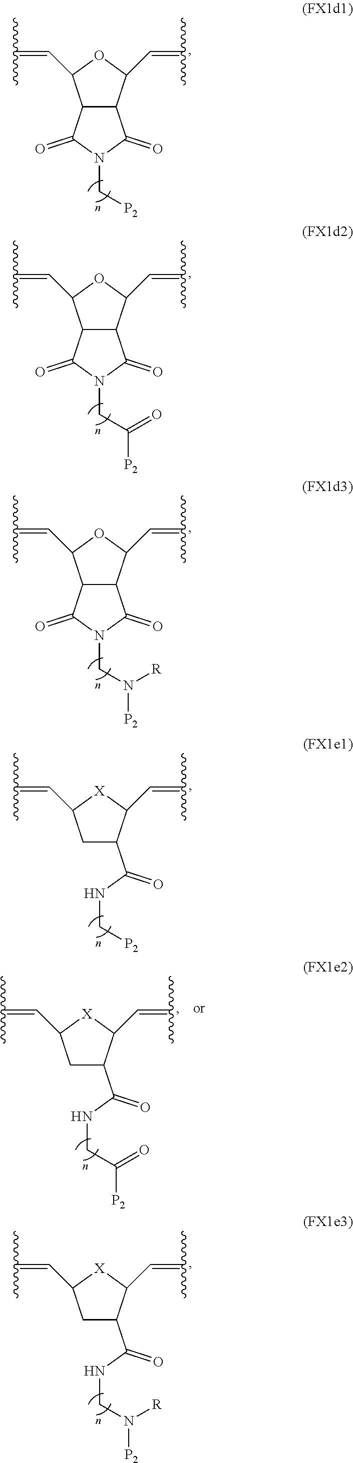

wherein n is an integer from 1 to 20 (e.g., 1, 2, 3, 4, 5, 6, 7, 8, 9, 10, 11, 12, 13, 14, 15, 16, 17, 18, 19, or 20), X is CH₂ or O, and R is hydrogen or a C₁-C₅ alkyl. In preferred embodiments, R is hydrogen. In other embodiments, the second backbone monomer is characterized by a formula (FX1e1), (FX1e2), or (FX1e3).

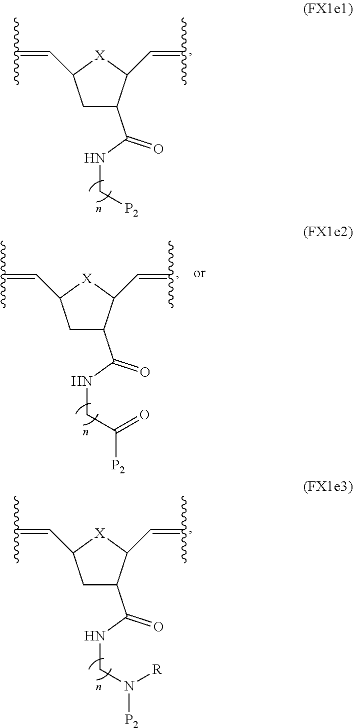

wherein n is an integer from 1 to 20 (e.g., 1, 2, 3, 4, 5, 6, 7, 8, 9, 10, 11, 12, 13, 14, 15, 16, 17, 18, 19, or 20), X is CH₂, and R is hydrogen.

In embodiments of the invention, the invention provides a block copolymer having a length from 1 nm to 20 nm. In some embodiments, the block copolymer has a length between 2 nm and 15 nm. In embodiments, the block copolymer may have less than or equal to 100 backbone monomers (e.g., less than or equal to 90 backbone monomers, less than or equal to 80 backbone monomers, less than or equal to 70 backbone monomers, less than or equal to 60 backbone monomers, less than or equal to 50 backbone monomers, less than or equal to 40 backbone monomers, less than or equal to 30 backbone monomers, less than or equal to 20 backbone monomers, less than or equal to 10 backbone monomers, less than or equal to 5 backbone monomers, or less than or equal to 2 backbone monomers). In some embodiments, the backbone monomer comprises less than or equal to 50 backbone monomers.

In some embodiments, the block copolymer polymer is compatible with renal clearance. In some embodiments, the block copolymer is characterized by an average molecular weight average less than 50 kDa, optionally less than or equal to 35 kDa, and optionally less than or equal to 20 kDa. In some embodiments, the block copolymer is characterized by an average molecular weight of 10 kDa to less than 50 kDa and optionally of 15 kDa to 35 kDa and optionally of 20 kDa to 35 kDa.

In some embodiments, the invention provides a block copolymer configured to deliver a therapeutic agent that is capable of treating, managing, stabilizing, relieving, healing, curing, and/or improving a condition of a subject. In aspects, the therapeutic agent may comprise an anti-inflammatory peptide, an anti-microbial peptide, a wound healing peptide, a myocardial infarction treatment peptide, or any combination thereof. In aspects, the therapeutic agent comprises a peptide for treatment of a neurodegenerative disease, inflammation, myocardial infarction, or any combination thereof.

In some aspects of the invention, the therapeutic agent comprises a peptide capable of treating MIs, peripheral neuropathies, and/or inflammation, such as a natriuretic peptide. In some aspects, the natriuretic peptide is naturally occurring. In other aspects, the natriuretic peptide is synthetic. In these aspects of the invention, the therapeutic agent has from 11 to 45 amino acid residues. For example, the therapeutic agent may comprise a sequence having 75% or greater sequence identity of any one of the sequence identities of SEQ ID NO: 172-SEQ ID NO: 183. In some aspects, the therapeutic agent may comprise SEQ ID NO: 225 (RPKPQQFFGLM) (i.e., Substance P) or SEQ ID NO: 223 (GYGSSSRRAPQT) (i.e., IGF-1C). For additional examples of therapeutic agents capable of treating MI reperfusion injury, see Fernandez Rico, et al., Front. Cardiovasc. Med. 9:792-885, 2022. The entire contents, including sequences, of each article are incorporated herein by reference in their entirety for all purposes.

Other examples of therapeutic agents capable of treating MIs, peripheral neuropathies, and/or inflammation include vascular endothelial growth factor (VEGF) peptides, Annexin-A1 N-Terminal peptide, and ghrelin peptides. Evidence of therapeutic effectiveness of these peptides are disclosed in Yang et al., J. Controlled Release, 213:27-35, 2015, Qin et al., Front. Pharmacol. 10:269, 2019, and Huang et al., Peptides, 30:2286-2291, 2009, respectively, the entire contents of each article are hereby incorporated by reference. In these examples of the invention, the therapeutic agent may comprise a sequence having 75% or greater sequence identity of any one of a (VEGF) peptide, an Annexin-A1 N-Terminal peptide, or a ghrelin peptide.

In an aspect, the invention provides a therapeutic agent comprising a therapeutic peptide having from 11 to 16 amino acid residues (e.g., 11 amino acid residues, 12 amino acid residues, 13 amino acid residues, 14 amino acid residues, 15 amino acid residues, or 16 amino acid residues) wherein the therapeutic agent further comprises a charge modulating domain having from 2 to 7 amino acid residues. In some further aspects, the therapeutic agent comprises a sequence having 85% or greater sequence identity of SEQ ID NO: 1 (LDEETGEFL), wherein the therapeutic agent further comprises a charge modulating domain having from 2 to 7 amino acid residues. In some embodiments, the therapeutic agent has 11 to 15 amino acid residues or the therapeutic agent has 12 to 14 amino acid residues.

As used herein, a sequence having 85% or greater sequence identity of SEQ ID NO: 1 (LDEETGEFL) indicates that the foregoing sequence can have one point mutation (i.e., amino acid change), one amino acid deletion, or one amino acid addition. In certain embodiments, the sequence having 85% or greater sequence identity of SEQ ID NO: 1 (LDEETGEFL) has a point mutation to comprise a proline residue. For example, any one of the glutamate residues can be changed to a proline residue. In some embodiments, the sequence having 85% or greater sequence identity of SEQ ID NO: 1 (LDEETGEFL) is SEQ ID NO: 1 (LDEETGEFL). In other embodiments, the sequence having 85% or greater sequence identity of SEQ ID NO: 1 (LDEETGEFL) is SEQ ID NO: 2 (LDPETGEFL).

In this aspect of the invention, the therapeutic agent comprises a charge modulating domain having from 2 to 7 amino acid residues. Typically, the charge modulating domain is a glycine-serine domain, a cationic residue domain, or a combination thereof. In certain embodiments, the charge modulating domain is a cationic residue domain having from 2 to 7 amino acid residues selected from lysine, arginine, histidine, and a combination thereof. In preferred embodiments, the charge modulating domain modulates the peptide to have a net positive charge. Without wishing to be bound by any particular theory, it is believed that the net positive charge increases the cellular uptake of the therapeutic agent or polymer comprising the therapeutic agent. Additionally, the addition of residues to form a net positive charge may enhance the aqueous solubility of the compound to facilitate therapeutic use.

In some embodiments, the therapeutic agent having from 11 to 16 amino acid residues comprises a sequence having 75% or greater (e.g., 80% or greater, 85% or greater, 90% or greater, 95% or greater, or 100%) sequence identity of SEQ ID NO: 1-SEQ ID: 138, SEQ ID: 140, or SEQ ID: 141. In some embodiments, the therapeutic agent having from 11 to 16 amino acid residues is selected from SEQ ID NO: 3-SEQ ID NO: 136, SEQ ID NO: 140, and SEQ ID NO: 141. In certain embodiments, the therapeutic agent has from 11 to 16 amino acid residues and is selected from SEQ ID NO: 3-SEQ ID NO: 136.

In another aspect, the invention provides a block copolymer comprising a first polymer segment comprising at least 2 first repeating units; wherein each of the first repeating units of the first polymers comprises a first backbone monomer group directly or indirectly covalently linked to a first polymer side chain group comprising a therapeutic agent; wherein the therapeutic agent comprises a sequence having 75% or greater (e.g., 80% or greater, 85% or greater, 90% or greater, 95% or greater, or 100%) sequence identity of SEQ ID NO: 1 (LDEETGEFL). The inventive polymer can be any suitable polymer type described herein and can comprise, or be derived from, any suitable number of monomers. For example, in some embodiments, the polymer is a homopolymer (i.e., derived from one type of monomer). Alternatively, in some embodiments, the polymer can be a copolymer comprising (e.g., derived from) more than one type of monomer (e.g., from 2 to 10 types of monomers). It will be understood that the inventive polymer, along with the linked polymer side chains, can have any suitable configuration. For example, in some embodiments wherein the polymer is a homopolymer, the polymer can be a brush polymer. In other embodiments wherein the polymer is a copolymer, the polymer can be a brush block copolymer or brush random copolymer.

In embodiments, the copolymer comprises a first polymer segment comprising from 1 to 1000 first repeating units. In other embodiments, the copolymer comprises a first polymer segment comprising at least 2 first repeating units, and optionally at least 5 first repeating units (e.g., 2-30, 5-30, 10-30, 15-30, or 20-30 first repeating units); wherein each of the first repeating units of the first polymer segment comprises a first backbone monomer group directly or indirectly covalently linked to a first polymer side chain group comprising a therapeutic agent comprising a sequence having 75% or greater sequence (e.g., 80% or greater, 85% or greater, 90% or greater, 95% or greater, or 100%) identity of SEQ ID NO: 1 (LDEETGEFL).

Thus, at least one polymer side chain (e.g., the first polymer segment) comprises a therapeutic agent. In some examples wherein the copolymer targets a protein-protein interaction between Nrf2 and Keap1, an inflammation site, or a MI reperfusion injury, the therapeutic agent comprises any suitable number of amino acid units so long as the peptide comprises a sequence having 75% or greater (e.g., 80% or greater, 85% or greater, 90% or greater, 95% or greater, or 100%) sequence identity of SEQ ID NO: 1 (LDEETGEFL). In keeping with an aspect of the invention, the therapeutic agent comprises at least 7 amino acid units. For example, the therapeutic agent comprises 7 or more amino acid units, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more amino acid units. Alternatively, or in addition, the therapeutic agent can comprise 100 or less amino acid units, for example, 90 or less, 80 or less, 70 or less, 60 or less, 59 or less, 58 or less, 57 or less, 56 or less, 55 or less, 54 or less, 53 or less, 52 or less, 51 or less, 50 or less, 49 or less, 48 or less, 47 or less, 46 or less, 45 or less, 44 or less, 43 or less, 42 or less, 41 or less, 40 or less, 39 or less, 38 or less, 37 or less, 36 or less, 35 or less, 34 or less 33 or less, 32 or less, or 31 or less amino acid units. Thus, the therapeutic agent can comprise a number of amino acid units bounded by any two of the aforementioned endpoints. For example, the therapeutic agent can comprise 7 to 100 amino acid units, for example, 7 to 90, 7 to 80, 7 to 70, 7 to 60, 7 to 50, 7 to 40, 7 to 30, 7 to 20, 7 to 16, 7 to 15, 7 to 14, 8 to 100, 8 to 90, 8 to 80, 8 to 70, 8 to 60, 8 to 50, 8 to 40, 8 to 30, 8 to 20, 8 to 16, 8 to 15, 8 to 14, 9 to 100, 9 to 90, 9 to 80, 9 to 70, 9 to 60, 9 to 50, 9 to 40, 9 to 30, 9 to 20, 9 to 16, 9 to 15, 9 to 14, 10 to 16, 10 to 15, 10 to 14, 11 to 16, 11 to 15, 11 to 14, 12 to 16, 12 to 15, or 12 to 14 amino acid units. In some embodiments, the therapeutic agent comprises 11 to 16 amino acids. In certain embodiments, the therapeutic agent comprises 11 to 15 amino acids or 12 to 14 amino acids.

In some embodiments, the therapeutic agent comprises a sequence having 85% or greater sequence identity of SEQ ID NO: 1 (LDEETGEFL). In certain embodiments, the therapeutic agent comprises a sequence having 75% or greater or 85% or greater sequence identity of SEQ ID NO: 1 (LDEETGEFL) has a point mutation to comprise a proline residue and/or a point mutation to delete a glutamate residue. In preferred embodiments, the peptide comprises SEQ ID NO: 1 (LDEETGEFL) or SEQ ID NO: 2 (LDPETGEFL). In other embodiments, the therapeutic agent comprises SEQ ID NO: 137 (LDPTGEFL) or SEQ ID NO: 138 (LDPETGFL).

In an embodiment, the therapeutic agent is selected from SEQ ID NO: 1-SEQ ID NO: 136, SEQ ID NO: 140, and SEQ ID NO: 141, optionally wherein the therapeutic agent is selected from SEQ ID NO: 3-SEQ ID NO: 136.

In embodiments, the copolymer regulates activity of MMP. In aspects of the invention, the therapeutic agent comprises a sequence having 75% or greater (e.g., 80% or greater, 85% or greater, 90% or greater, 95% or greater, or 100%) sequence identity of any one of SEQ ID NO: 156-SEQ ID NO: 171. In some aspects of the invention, the therapeutic agent comprises a sequence having 75% or greater (e.g., 80% or greater, 85% or greater, 90% or greater, 95% or greater, or 100%) sequence identity of TIMP-1. For additional information on MMP regulatory molecules, selective inhibitors in particular, including characterizations and sequences thereof, see Ndinguri et al., Molecules, 17: 14230-14248 (2012), which is hereby incorporated by reference in its entirety.

In some embodiments, the therapeutic agent comprising a sequence having 75% or greater (e.g., 80% or greater, 85% or greater, 90% or greater, 95% or greater, or 100%) sequence identity of SEQ ID NO: 165 (VMDGYPMP) or SEQ ID NO: 166 (GYPKSALR) may further comprise an acetyl group on the N-terminus and an amino group on the C-terminus of the sequence.

The therapeutic agent can have any suitable structure (e.g., primary, secondary, tertiary, or quaternary structure) described herein. The therapeutic agent can be a branched peptide, a linear peptide, cyclic peptide, or a cross-linked peptide. In some embodiments, the copolymer is characterized by a structure wherein at least a portion of the therapeutic agent is linked to the copolymer backbone group via an enzymatically degradable linker, such a matrix metalloproteinase (MMP) cleavage sequence, cathepsin B cleavage sequence, ester bond, reductive sensitive bond-disulfide bond, pH sensitive bond-imine bond or any combinations of these. In other embodiments, the copolymer is characterized by a structure wherein at least a portion of the therapeutic agent side-chain is linked to the copolymer backbone or consists of a degradable or triggerable linker. In some embodiments, the therapeutic agent and/or copolymer further comprises a tag for imaging and/or analysis. For example, the therapeutic agent and/or copolymer can further comprise a dye, radiolabeling, an imaging agent, tritiation, and the like.

In aspects of the invention, the therapeutic agent may further comprise a small molecule therapeutic wherein at least a portion of the therapeutic agent side-chain is linked to the small molecule therapeutic. For example, the small molecule therapeutic may comprise a known MMP regulator, such as PD-166793 or doxycycline. In other examples, the therapeutic agent further comprises an additional Keap1 inhibitor or Nrf2 inducer, such as dimethyl fumarate, tert-butylhydroquinone, DL-sulforaphane, or the like. Other small molecule MMP regulators, Keap1 inhibitors, or Nrf2 inducers will be readily apparent to those skill in the art. In certain embodiments, the therapeutic agent further comprises an additional Keap1 inhibiting peptide. In other words, the block copolymer can comprise a protein-like polymer described herein and an additional peptide.

In some embodiments, the therapeutic agent further comprises a charge modulating domain. The charge modulating domain can be any suitable amino acid domain, which increases the positive charge of the therapeutic agent. For example, the charge modulating domain can be a TAT sequence, a glycine-serine domain, a cationic residue domain, or a combination thereof. In some embodiments, the charge modulating domain is a glycine-serine domain, a cationic residue domain, or a combination thereof. In certain embodiments, the charge modulating domain is a cationic residue domain having from 2 to 7 amino acid residues selected from lysine, arginine, histidine, and a combination thereof. In preferred embodiments, the charge modulating domain modulates the therapeutic agent to have a net positive charge.

The present invention provides a modifiable drug delivery system capable of housing a variety of known therapeutic agents, not merely the sequences and peptides disclosed herein. The present disclosure allows a skilled artisan to modify the inventive block copolymer with virtually any therapeutic agent, provided that said therapeutic agent is structurally compatible with the block copolymer and comprises a number of amino acid units between the range of 3 to 150 amino acid units, optionally 5 to 50 amino acid units.

In another aspect, the invention provides a block copolymer comprising a second polymer segment comprising from 1 to 1000 second repeating units; wherein each of the second repeating units of the second polymers comprises a second backbone monomer group directly or indirectly covalently linked to a second polymer side chain group comprising a targeting agent.

In other embodiments, the copolymer comprises a second polym

er segment comprising at least 2 second repeating units, and optionally at least 5 second repeating units (e.g., 2-30, 5-30, 10-30, 15-30, or 20-30 first repeating units); wherein each of the second repeating units of the second polymer segment comprises a second backbone monomer group directly or indirectly covalently linked to a second polymer side chain group comprising a targeting agent.

In some aspects of the invention, the targeting agent comprises a synthetic peptide or a recombinant peptide. In other aspects, the targeting agent comprises a naturally occurring peptide. The targeting agent comprises an interleukin-6 inhibitor, an interleukin-10 inhibitor, a JAK/STAT inhibitor, or a LOX inhibitor in some aspects of the invention.

In embodiments, the targeting agent comprises a sequence having 75% or greater (e.g., 80% or greater, 85% or greater, 90% or greater, 95% or greater, or 100%) sequence identity of SEQ ID NO: 142 (AAKAAKAA) or SEQ ID NO: 150 (GGGGEKGGGGG).

Thus, at least one polymer side chain (e.g., the second polymer segment) comprises a targeting agent. In some examples of the invention targeting the LOX enzyme, the targeting agent comprises any suitable number of amino acid units so long as the targeting agent comprises a sequence having 75% or greater (e.g., 80% or greater, 85% or greater, 90% or greater, 95% or greater, or 100%) sequence identity of SEQ ID NO: 142 (AAKAAKAA). In other examples of the invention targeting the LOX enzyme, the targeting agent comprises any suitable number of amino acid units so long as the targeting agent comprises a sequence having 75% or greater (e.g., 80% or greater, 85% or greater, 90% or greater, 95% or greater, or 100%) sequence identity of SEQ ID NO: 150 (GGGGEKGGGGG).

In certain embodiments, the targeting agent comprises a sequence having 75% or greater or 85% or greater sequence identity of SEQ ID NO: 142 (AAKAAKAA) has a point mutation to comprise an arginine residue and/or a point mutation to comprise a glutamate residue.

In certain embodiments, the targeting agent comprises a sequence having 75% or greater or 85% or greater sequence identity of SEQ ID NO: 150 (GGGGEKGGGGG) has a point mutation to comprise a glutamine residue.

In keeping with the aspects of the invention targeting the LOX enzyme, the targeting agent comprises at least one amino acid residue capable of binding to calcium. The targeting agent preferably comprises a crosslinkable amino acid residue, such as lysine or methylated lysine. In further aspects, the targeting agent comprises an amino acid sequence having a length selected from the range of 4.8 Å to 14.2 Å to correspond to substrate configured to bind with a LOX enzyme at a target site. Without wishing to be bound by any particular theory, it is believed that the LOX enzymatic structure has a maximum binding site length of 14.2 Å in its open state conformation and has a minimum functional domain of 4.8 Å in its closed state conformation.

In some examples of invention targeting the MMP enzyme, the targeting agent comprises any suitable number of amino acid units so long as the targeting agent comprises a sequence having 75% or greater (e.g., 80% or greater, 85% or greater, 90% or greater, 95% or greater, or 100%) sequence identity of SEQ ID NO: 154 (GPLGLAGGWGERDGS). In other examples, the targeting agent comprises any suitable number of amino acid units so long as the targeting agent comprises a sequence having 75% or greater (e.g., 80% or greater, 85% or greater, 90% or greater, 95% or greater, or 100%) sequence identity of SEQ ID NO: 155 (GGSGSGSGWGERDGS).

In an aspect of the invention, the targeting agent comprises at least 3 amino acid units. For example, the peptide comprises 3 or more amino acid units, 4 or more amino acid units, 5 or more amino acid units, 6 or more amino acid units, 7 or more amino acid units, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more amino acid units. Alternatively, or in addition, the peptide can comprise 100 or less amino acid units, for example, 90 or less, 80 or less, 70 or less, 60 or less, 59 or less, 58 or less, 57 or less, 56 or less, 55 or less, 54 or less, 53 or less, 52 or less, 51 or less, 50 or less, 49 or less, 48 or less, 47 or less, 46 or less, 45 or less, 44 or less, 43 or less, 42 or less, 41 or less, 40 or less, 39 or less, 38 or less, 37 or less, 36 or less, 35 or less, 34 or less 33 or less, 32 or less, or 31 or less amino acid units. Thus, the targeting agent can comprise a number of amino acid units bounded by any two of the aforementioned endpoints. For example, the targeting agent can comprise 3 to 100 amino acid units, for example, 3 to 90, 3 to 80, 3 to 70, 3 to 60, 3 to 50, 3 to 40, 3 to 30, 3 to 20, 3 to 16, 3 to 15, 3 to 14, 4 to 90, 4 to 80, 4 to 70, 4 to 60, 4 to 50, 4 to 40, 4 to 30, 4 to 20, 4 to 16, 4 to 15, 4 to 14, 5 to 90, 5 to 80, 5 to 70, 5 to 60, 5 to 50, 5 to 40, 5 to 30, 5 to 20, 5 to 16, 5 to 15, 5 to 14, 6 to 90, 6 to 80, 6 to 70, 6 to 60, 6 to 50, 6 to 40, 6 to 30, 6 to 20, 6 to 16, 6 to 15, 6 to 14, 7 to 90, 7 to 80, 7 to 70, 7 to 60, 7 to 50, 7 to 40, 7 to 30, 7 to 20, 7 to 16, 7 to 15, 7 to 14, 8 to 100, 8 to 90, 8 to 80, 8 to 70, 8 to 60, 8 to 50, 8 to 40, 8 to 30, 8 to 20, 8 to 16, 8 to 15, 8 to 14, 9 to 100, 9 to 90, 9 to 80, 9 to 70, 9 to 60, 9 to 50, 9 to 40, 9 to 30, 9 to 20, 9 to 16, 9 to 15, 9 to 14, 10 to 16, 10 to 15, 10 to 14, 11 to 16, 11 to 15, 11 to 14, 12 to 16, 12 to 15, or 12 to 14 amino acid units. In some embodiments, the therapeutic agent comprises 11 to 16 amino acids. In certain embodiments, the targeting agent comprises 11 to 15 amino acids or 12 to 14 amino acids.

The targeting agent can have any suitable structure (e.g., primary, secondary, tertiary, or quaternary structure) described herein. The targeting agent can be a branched peptide, a linear peptide, cyclic peptide, or a cross-linked peptide. In some embodiments, the polymer is characterized by a structure wherein at least a portion of the peptide is linked to the polymer backbone group via an enzymatically degradable linker, such a matrix metalloproteinase (MMP) cleavage sequence, a lysl oxidase (LOX) cleavage sequence, cathepsin B cleavage sequence, ester bond, reductive sensitive bond-disulfide bond, pH sensitive bond-imine bond or any combinations of these. In other embodiments, the polymer is characterized by a structure wherein at least a portion of the targeting agent side-chain is linked to the polymer backbone or consists of a degradable or triggerable linker. In some embodiments, the targeting agent and/or copolymer further comprises a tag for imaging and/or analysis. For example, the targeting agent and/or copolymer can further comprise a dye (e.g., cyanine5.5 fluorescent dye), radiolabeling, an imaging agent, tritiation, and the like.

In some embodiments, the targeting agent further comprises a charge modulating domain. The charge modulating domain can be any suitable amino acid domain, which increases the positive charge of the targeting agent. In some embodiments, the charge modulating domain is a glycine-serine domain, a cationic residue domain, or a combination thereof. In certain embodiments, the charge modulating domain is a cationic residue domain having from 2 to 7 amino acid residues selected from lysine, arginine, histidine, and a combination thereof. In preferred embodiments, the charge modulating domain modulates the targeting agent to have a net positive charge.

In some embodiments, the targeting agent comprises a sequence having 85% or greater sequence identity of SEQ ID NO: 142 (AAKAAKAA) or SEQ ID NO: 150 (GGGGEKGGGGG). In preferred embodiments, the targeting agent comprises SEQ ID NO: 142 (AAKAAKAA). In other embodiments, the targeting agent is selected from SEQ ID NO: 143-SEQ ID NO: 149. In other embodiments, the targeting agent is selected from SEQ ID NO: 151-SEQ ID NO: 153 and of SEQ ID NO: 189 (GGGDQKGGGGG), SEQ ID NO: 190 (GGGDPKGGGGG), or SEQ ID NO: 191 (GGGQEKGGGGG).

In other embodiments wherein the targeting agent is configured to target the LOX enzyme, the targeting agent comprises a sequence having 75% or greater sequence identity of any one of SEQ ID NO: 195 (ELSYGYDEKSTG)-SEQ ID NO: 210 (VGGEKSG).

In other embodiments wherein the targeting agent is configured to target the LOX enzyme, the targeting agent comprises a sequence having 75% or greater sequence identity of any one of (IKG), (AKG), or SEQ ID NO: 213 (AKGS) wherein the first amino acid of SEQ ID NO: 213 is optionally attached to an N-acetyl group. In embodiments, IKG and/or AKG optionally have a butoxycarbonyl protecting group (Boc) or a tert (t)-Boc attached to the first amino acid of the corresponding sequence. Additional information on these sequences, including conformational features, spectroscopic data, and their use as substrates for LOX enzymes, is provided in Jiang & Ananthanarayanan, J. Biolog. Chem. 266: 22960-22967, 1991, which is hereby incorporated by reference.

Additional examples of targeting agents configured to target LOX include a targeting agent comprising at least a portion of random co-polymers of lysine. For example, the targeting agent may comprise a sequence having 75% or greater sequence identity of SEQ ID NO: 184 (AKAK); SEQ ID NO: 185 (VKVK); SEQ ID NO: 186 (KKKK); or SEQ ID NO: 187 (LKLK), wherein the third and fourth amino acid residues of each sequence identity are optionally repeated between 0 and 49 times. For example, SEQ ID NO: 184 can also be described with the formula: AK(AK)h, wherein h is an integer selected from the range of 0 to 49. For additional information on these random co-polymers of lysine, including their rates of oxidation by LOX enzyme, see Kagan et al., J. Biolog. Chem. 259:11203-11207, 1984, which is hereby incorporated by reference.

In embodiments, the targeting agent may comprise at least a portion of repeat polypeptide models of elastin to target the LOX enzyme. For example, the targeting agent may comprise a sequence having 75% or greater sequence identity of any one of SEQ ID NO: 217 (VPGGV)-SEQ ID NO: 222 (VAPGKGV). In examples, the targeting agent may comprise one or more of the following peptides: (ΦPGG)n-V; (VPGΦG)n-V; or V(APGΦGV)n provided that each (independently comprises V or K at approximately a 4:1 ratio; the first amino acid residue is optionally attached to a carbonyl group; the last amino acid residue is optionally attached to a methoxy group; and each n is an integer having a value greater than or equal to 40. For example the targeting agent may comprise a peptide having the sequence identity of SEQ ID NO: 226, which corresponds to: (VPGG)₄₀(KPGG)₁₀-V, wherein the first amino acid residue is optionally attached to a carbonyl group and the last amino acid residue is optionally attached to a methoxy group. Additional information on polypeptide models of elastin, including sequences and interactions with LOX enzymes is provided in Kagan et al., J. Biolog. Chem. 255:3658-36559, 1980, which is hereby incorporated by reference.

In some embodiments of the invention targeting heart tissue of a subject, the targeting agent comprises any suitable number of amino acid units so long as the targeting agent comprises a sequence having 75% or greater (e.g., 80% or greater, 85% or greater, 90% or greater, 95% or greater, or 100%) sequence identity of SEQ ID NO: 214 (CSTSMLKAC), SEQ ID NO: 215 (CKPGTSSYC), or SEQ ID NO: 216 (CPDRSVNNC). Additional information on SEQ ID NO: 214-SEQ ID NO: 216, such as their capability to selectively target for ischemic myocardium, see Kanki et al., J. Mol. Cell Cardiol. 50(5): 841-848, 2011, which is hereby incorporated by reference.

The present invention provides a modifiable drug delivery system capable of housing a variety of known targeting agents, not merely the sequences and peptides disclosed herein. The present disclosure allows a skilled artisan to modify the inventive block copolymer with virtually any targeting agent, provided that said targeting agent is structurally compatible with the block copolymer and comprises a number of amino acid units between the range of 3 to 150 amino acid units, optionally 5 to 50 amino acid units.

In some embodiments, the targeting agent facilitates the aggregation and/or localization of the block copolymer at a target site of a subject. In examples, the aggregation and/or localization is the result of the formation of cross-linkages between the targeting agent and tissue at the target site of the subject. Alternatively, or additionally, aggregation and/or localization is the result of the formation of cross-linkages of the targeting agent with itself and/or with portions of the block copolymer. In examples, the cross-linkages involving the targeting agent are facilitated by a cross-linking enzyme at the target site. In some embodiments, the cross-linkages are comprised of disulfide bonds.

In an aspect of the invention, the aggregation and/or localization of the block copolymer at a target site of a subject is facilitated by the block copolymer's high renal clearance rate. Without subscribing to a particular theory, it is believed that the chemical structure of the block copolymer facilitates the high renal clearance rate which allows for superior targeting and competitive accumulation.

In another aspect, the invention provides a pharmaceutical composition comprising one or more therapeutic agents and/or one or more block copolymers described herein. In some embodiments, the composition comprises one or more pharmaceutically acceptable excipients. For example, the therapeutic agents and/or copolymers of the invention can be formulated for parenteral administration, such as intravenous (IV) administration or administration into a body cavity or lumen of an organ. Alternatively, the therapeutic agents and/or copolymers can be injected intra-tumorally. Formulations for injection will commonly comprise a solution of the therapeutic agent and/or copolymer dissolved in a pharmaceutically acceptable carrier. Among the acceptable vehicles and solvents that can be employed are water and an isotonic sodium chloride. In addition, sterile fixed oils can conventionally be employed as a solvent or suspending medium. For this purpose, any bland fixed oil can be employed including synthetic monoglycerides or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the therapeutic agent and/or copolymer in these formulations can vary widely, and will be selected primarily based on fluid volumes, viscosities, body weight, and the like, in accordance with the particular mode of administration selected and the patient's needs. In certain embodiments, the concentration of a therapeutic agent and/or copolymer in a solution formulation for injection will range from 0.1% (w/w) to 10% (w/w) or about 0.10% (w/w) to about 10% (w/w).

In another aspect, the invention provides a method of treating or managing a condition comprising administering to a subject an effective amount of a peptide, copolymer, and/or pharmaceutical composition described herein. The peptide, copolymer, and/or pharmaceutical composition can be administered by oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. In some embodiments, the peptide, copolymer, and/or pharmaceutical composition is administered intravenously, subcutaneously, intramuscularly, topically, orally, or a combination thereof.

The methods described herein can comprise contacting a target tissue of the subject with the therapeutic agent and/or copolymer or a metabolite or product thereof, contacting a target cell of the subject with the therapeutic agent and/or copolymer or a metabolite or product thereof, and/or contacting a target receptor of the subject with the peptide and/or polymer or a metabolite or product thereof. In preferred embodiments, the therapeutic agents and/or copolymers described herein pass through the cell membrane and contact an intracellular target. Without wishing to be bound by any particular theory, it is believe that the peptide/polymer structure and charge described herein play an integral role in providing cell permeability.

In some embodiments, the methods described herein interrupt the protein-protein interaction between Nuclear factor (erythroid-derived 2)-like 2 (Nrf2) and Kelch-like ECH-Associating protein 1 (Keap1) (e.g., by inhibiting binding to the ETGE motif of Nrf2). Without wishing to be bound by any particular theory, inhibiting Keap1/Nrf2 binding can enhance the antioxidant and anti-inflammatory response to provide beneficial effects in both the central nervous system (CNS) and/or the non-central nervous system. Thus, the methods described herein can be used to treat and/or manage a condition associated with an inflammatory state, increased oxidative stress, autoimmune pathophysiology, chemo-preventative measures, neurodegeneration, or a combination thereof.

The method includes administering a therapeutically effective amount of a peptide, polymer, and/or composition described herein to a subject in need thereof. For example, the methods can include administering the therapeutic agent, copolymer, and/or composition to provide a dose of from 10 ng/kg to 50 mg/kg to the subject. For example, the therapeutic agent and/or copolymer dose can range from 5 mg/kg to 50 mg/kg, from 10 μg/kg to 5 mg/kg, or from 100 μg/kg to 1 mg/kg. The therapeutic agent and/or copolymer dose can also lie outside of these ranges, depending on the particular peptide and/or polymer as well as the type of disease being treated. Frequency of administration can range from a single dose to multiple doses per week, or more frequently. In some embodiments, the therapeutic agent and/or copolymer is administered from once per month to five times per week. In some embodiments, the peptide and/or polymer is administered once per week.

In some embodiments, the methods described herein can be used to treat or manage an autoimmune disease. For example, the methods described herein can be used to treat or manage multiple sclerosis, systemic lupus erythematous, Sjogren syndrome, rheumatoid arthritis, vitiligo, psoriasis, or the like.

In some embodiments, the methods described herein can be used to treat or manage a respiratory disease. For example, the methods described herein can be used to treat or manage COPD, emphysema, potential treatment for smokers, idiopathic pulmonary fibrosis, chronic sarcoidosis, hypersensitivity pneumonitis, or the like.

In some embodiments, the methods described herein can be used to treat or manage a gastrointestinal disease. For example, the methods described herein can be used to treat or manage ulcerative colitis, ulcers, prevent acetaminophen toxicity, non-alcoholic steatohepatitis, primary biliary cholangitis, cirrhosis, type 2 diabetes, diabetic nephropathy, or the like.

In some embodiments, the methods described herein can be used to treat or manage a cardiovascular disease. For example, the methods described herein can be used to treat or manage cardiac ischemia-reperfusion injury, heart failure, atherosclerosis, or the like.

In some embodiments, the methods described herein can be used to treat or manage a neurodegenerative disease. For example, the methods described herein can be used to treat or manage Alzheimer's disease, Parkinson's disease, amyotrophic lateral sclerosis (ALS), Huntington's disease, Friedreich ataxia, frontotemporal lobar degeneration, or the like. The invention may be further set forth and understood in view of the following non-limiting examples and embodiments which one having skill in the art will readily understand are intended to illustrate specific aspects of the invention.

### ASPECTS OF THE INVENTION

Various aspects are contemplated herein, several of which are set forth in the paragraphs below. It is explicitly contemplated that any aspect or portion thereof can be combined to form an aspect. In addition, it is explicitly contemplated that any aspect (e.g., Aspect A13) that references an aspect (e.g., Aspect A1) for which there are sub-aspects having the same top level number (e.g., Aspect A1a, A1b, A1c, and so forth) necessarily includes reference to those sub-aspects A1a, A1b, A1c, and so forth. In other words, if Aspect A13 refers to Aspect A1, and there are Aspects A1a and A1b present, then Aspect A13 refers to Aspects A1a or A1b. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspects B1-B12, or any preceding aspect, . . . ” means that any aspect prior to aspect B13 is referenced, including aspects B1-B12 and all of the “A” aspects). For example, it is contemplated that, optionally, any method or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y, even through “less than Y” in isolation does not include Y.

Aspect A1: A block copolymer characterized by a formula (FX1a):

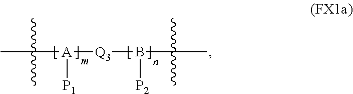

wherein
each A is independently a first backbone monomer;
each B is independently a second backbone monomer, wherein B is more hydrophilic than A;
m is an integer selected from the range of 1 to 1000 (e.g., 1 to 500, 1 to 250, 1 to 100, 1 to 50, 1 to 20, or 1 to 10);
n is an integer selected from the range of 1 to 1000 (e.g., 1 to 500, 1 to 250, 1 to 100, 1 to 50, 1 to 20, or 1 to 10);
Q₃ is optionally present and is a linking group (e.g., single bond;, —O—, or a substituted or unsubstituted C₁-C₂₀ alkylene, C₂-C₂₀ heteroalkylene, C₃-C₂₀ cycloalkylene, C₃-C₂₀ arylene, C₃-C₂₀ heteroarylene, C₂-C₂₀ alkenylene, C₂-C₂₀ cycloalkenylene, C₁-C₂₀ alkoxy, C₁-C₁₀ acyl; one or more methylacrylamide or acrylamide backbone monomers), optionally a polymer grafting group (e.g., one or more backbone monomer such as substitute or unsubstituted norbornene such as norbornene imide, norbornene amide); optionally a dye, chromaphore, flourophor or other probe, visualization or imaging agent.)
each P₁ is optionally present and is a therapeutic agent, optionally a therapeutic peptide, provided that at least one P₁ is present, and optionally a plurality of P₁ is present; and
each P₂ is optionally present and is a targeting agent, optionally a targeting peptide, provided that at least one P₂ is present, and optionally a plurality of P₂ is present.

Aspect A2: The block copolymer of aspect A1, wherein the block copolymer is characterized by a formula (FX1b):

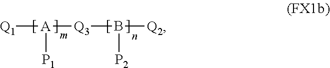

wherein Q₁ and Q₂ are each independently a polymer block terminating group.

Aspect A3: The block copolymer of aspect A1 or aspect A2, wherein monomer [A(P₁)] is characterized by a formula (FX1c):

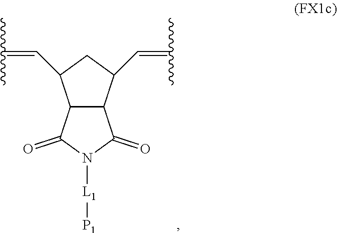

wherein L₁ is optionally present and is a first linking group.

Aspect A4: The block copolymer of any one of aspects A1-A3, wherein monomer [B(P₂)] is characterized by a formula (FX1d) or (FX1e):

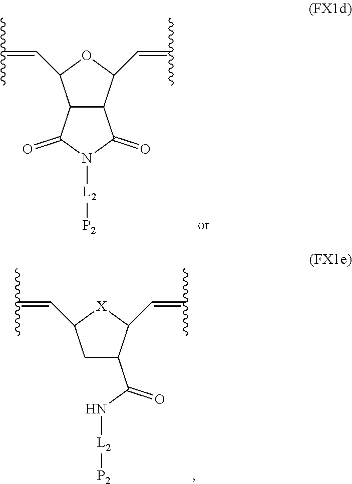

wherein L₂ is optionally present and is a second linking group, and X is CH₂ or O.

Aspect A5: The block copolymer of any one of aspects A1-A4, wherein monomer [B(P₂)] is characterized by a formula (FX1e):

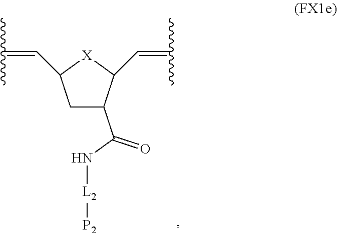

wherein X is CH₂.

Aspect A6: The block copolymer of any one of aspects A1-A5, wherein the block copolymer comprises a first backbone monomer characterized by a formula (FX1c1), (FX1c2), or (FX1c3):

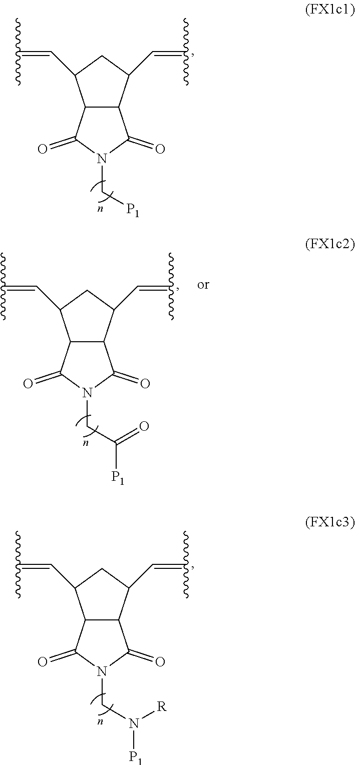

wherein n is an integer from 1 to 20 (e.g., 1, 2, 3, 4, 5, 6, 7, 8, 9, 10, 11, 12, 13, 14, 15, 16, 17, 18, 19, or 20) and R is hydrogen or a C₁-C₅ alkyl.

Aspect A6a: The block copolymer of any one of aspects A1-A6, wherein the block copolymer comprises a first backbone monomer characterized by a formula (FX1c1), (FX1c2), or (FX1c3), wherein n is an integer from 1 to 20 (e.g., 1, 2, 3, 4, 5, 6, 7, 8, 9, 10, 11, 12, 13, 14, 15, 16, 17, 18, 19, or 20) and R is hydrogen.

Aspect A7: The block copolymer of any one of aspects A1-A6a, wherein the block copolymer comprises a second backbone monomer characterized by a formula (FX1d1), (FX1d2), (FX1d3), (FX1e1), (FX1e2), or (FX1e3):

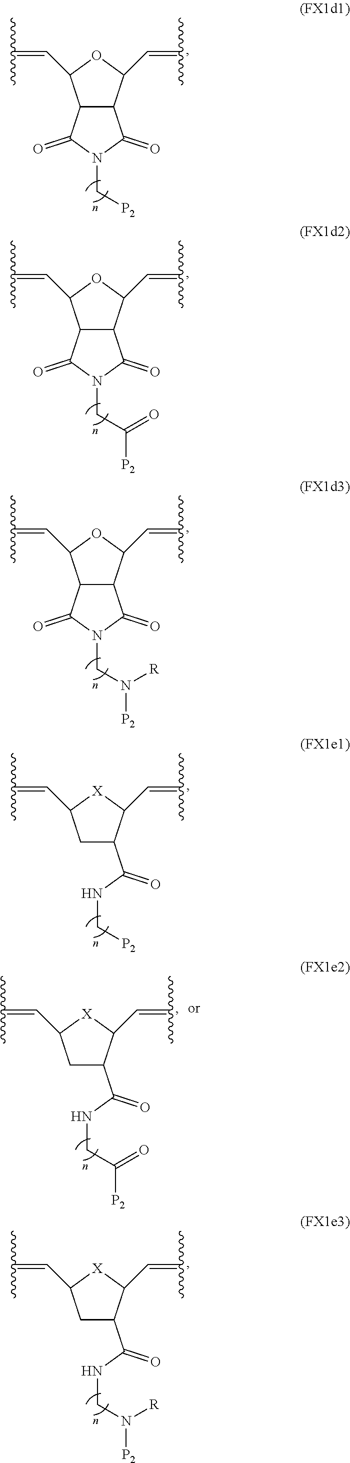

wherein n is an integer from 1 to 20 (e.g., 1, 2, 3, 4, 5, 6, 7, 8, 9, 10, 11, 12, 13, 14, 15, 16, 17, 18, 19, or 20), X is CH₂ or O, and R is hydrogen or a C₁-C₅ alkyl.

Aspect A7a. The block copolymer of any one of aspects A1-A7, wherein the block copolymer comprises a second backbone monomer characterized by a formula (FX1d1), (FX1d2), (FX1d3), (FX1e1), (FX1e2), or (FX1e3), wherein n is an integer from 1 to 20 (e.g., 1, 2, 3, 4, 5, 6, 7, 8, 9, 10, 11, 12, 13, 14, 15, 16, 17, 18, 19, or 20) and R is hydrogen.

Aspect A8: The block copolymer of any one of aspects A1-A7a, wherein the second backbone monomer is characterized by a formula (FX1e1), (FX1e2), or (FX1e3):

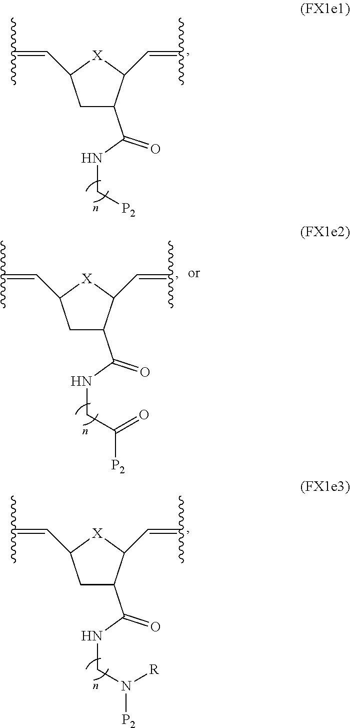

wherein n is an integer from 1 to 20 (e.g., 1, 2, 3, 4, 5, 6, 7, 8, 9, 10, 11, 12, 13, 14, 15, 16, 17, 18, 19, or 20), X is CH₂, and R is hydrogen.

Aspect A9: The block copolymer of any one of aspects A3-A5, or any preceding aspect, wherein each of L₁ and L₂ is independently selected from a single bond, an oxygen, and groups having an alkylene group, a heteroalkylene group, an alkenylene group, an arylene group, an alkoxy group, an acyl group, a triazole group, a diazole group, a pyrazole group, and combinations thereof.

Aspect A10: The block copolymer of any one of aspects A3-A5, or any preceding aspect, wherein each of L₁ and L₂ is independently selected from a single bond, O—, C₁-C₁₀ alkyl, C₂-C₁₀ alkylene, C₁-C₁₀ heteroalkylene, C₃-C₁₀ arylene, C₁-C₁₀ alkoxy, C₁-C₁₀ acyl and combinations thereof.

Aspect A11: The block copolymer of any one of aspects A3-A5, or any preceding aspect, wherein each of L₁ and L₂ is independently selected from C₁-C₂₀ alkylene and C₁-C₂₀ heteroalkylene, optionally terminated in a carbonyl, an amine, or an amide.

Aspect A12: The block copolymer of any one of aspects A3-A5, or any preceding aspect, wherein each of L₁ and L₂ is independently selected from —(CH₂)ₙNR—, —(CH₂)ₙC(O)NR—, —(CH₂)ₙNRC(O)—, —(CH₂)ₙC(O)— and —(CH₂)ₙ—, wherein n is an integer from 1 to 20 (e.g., 1, 2, 3, 4, 5, 6, 7, 8, 9, 10, 11, 12, 13, 14, 15, 16, 17, 18, 19, or 20) and R is hydrogen or a C₁-C₅ alkyl.

Aspect A12a: The block copolymer of aspect A12, or any preceding aspect, wherein each of L₁ and L₂ is independently selected from —(CH₂)ₙNR—, —(CH₂)ₙC(O)NR—, —(CH₂)ₙNRC(O)—, —(CH₂)ₙC(O)— and —(CH₂)ₙ—, wherein n is an integer from 1 to 20 (e.g., 1, 2, 3, 4, 5, 6, 7, 8, 9, 10, 11, 12, 13, 14, 15, 16, 17, 18, 19, or 20) and R is hydrogen.

Aspect A13: The block copolymer of aspect A12 or A12a, or any preceding aspect, wherein n is an integer from 1 to 10 (e.g., 1, 2, 3, 4, 5, 6, 7, 8, 9, or 10) and R is hydrogen.

Aspect A14: The block copolymer of any one of aspects A3-A13, or any preceding aspect, wherein each of L₁ and/or L₂ further comprises an enzymatically degradable linker, and wherein at least a portion of each of P₁ and P₂ is independently linked to the enzymatically degradable linker.

Aspect A15: The block copolymer of aspect A14, or any preceding aspect, wherein the enzymatically degradable linker is a MMP cleavage sequence, a cathepsin B cleavage sequence, an ester bond, a reductive sensitive bond-disulfide bond, a pH sensitive bond-imine bond, or any combination thereof.

Aspect A16: The block copolymer of aspect A15, or any preceding aspect, wherein the enzymatically degradable linker is a carbamate.

Aspect A17: The block copolymer of any one of aspects A1-A16, wherein each of Q₁ and Q₂ is independently selected from a hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ is independently H, C₅-C₁₀ aryl or C₁-C₁₀ alkyl.

Aspect A18: The block copolymer of any one of aspects A2-A17, or any preceding aspect, wherein each of Q₁ and Q₂ is independently selected from a hydrogen, an amino, an acetyl, or a phenyl.

Aspect A19: The block copolymer of any one of aspects A2-A18, wherein Q₃ is present and comprises a dye.

Aspect A20: The block copolymer of aspect A19, or any preceding aspect, wherein the dye is a Cyanine5.5 fluorescent dye.

Aspect A21: The block copolymer of any one of aspects A1-A20, wherein the block copolymer has an average degree of polymerization of 2 to 1000 (e.g., 2 to 500, 2 to 250, 2 to 100, 2 to 50, 2 to 30, 5 to 1000, 5 to 500, 5 to 250, 5 to 100, 5 to 50, 5 to 30, 20 to 500, 20 to 250, 20 to 100, 20 to 50, or 20 to 30).

Aspect A22: The block copolymer of any one of aspects A1-A21, wherein the block copolymer has an average degree of polymerization of 2 to 100 (e.g., 2 to 100, 2 to 90, 2 to 80, 2 to 70, 2 to 60, 2 to 50, 2 to 40, 2 to 30, 2 to 20, 2 to 10, 4 to 100, 4 to 90, 4 to 80, 4 to 70, 4 to 60, 4 to 50, 4 to 40, 4 to 30, 4 to 20, 4 to 10, 10 to 50, 10 to 40, 10 to 30, 20 to 50, 20 to 40, or 20 to 30).

Aspect A23: The block copolymer of any one of aspects A1-A22, wherein the block copolymer has an average degree of polymerization of 2 to 20 (e.g., 2 to 20, 2 to 16, 2 to 12, 2 to 8, 2 to 4, 3 to 20, 3 to 16, 3 to 12, 3 to 8, 3 to 4, 4 to 20, 4 to 16, 4 to 12, 4 to 8, 5 to 20, 5 to 16, 5 to 15, 5 to 12, 5 to 10, 5 to 8, 6 to 20, 6 to 16, 6 to 12, 6 to 8, 7 to 20, 7 to 16, 7 to 14, 7 to 12, 7 to 8; 8 to 20, 8 to 16, 8 to 12, 8 to 10, 9 to 20, 9 to 18, 9 to 16, 9 to 12, 9 to 10, 10 to 20, 10 to 18, 10 to 16, 10 to 15, 10 to 14, 10 to 13, 10 to 12, or 10 to 11).

Aspect A24: The block copolymer of any one of aspects A1-A23, wherein the block copolymer has an average molecular weight of 5 kDa to less than 50 kDa (e.g., 5 kDa to 50 kDa, 5 kDa to 45 kDa, 5 kDa to 40 kDa, 5 kDa to 35 kDa, 5 kDa to 30 kDa, 5 kDa to 25 kDa, 5 kDa to 20 kDa, 5 kDa to 15 kDa, 5 kDa to 10 kDa, 7 kDa to 50 kDa, 7 kDa to 45 kDa, 7 kDa to 40 kDa, 7 kDa to 35 kDa, 7 kDa to 30 kDa, 7 kDa to 25 kDa, 7 kDa to 20 kDa, 7 kDa to 15 kDa, 7 kDa to 10 kDa, 10 kDa to 50 kDa, 10 kDa to 45 kDa, 10 kDa to 40 kDa, 10 kDa to 35 kDa, 10 kDa to 30 kDa, 10 kDa to 25 kDa, 10 kDa to 20 kDa, 10 kDa to 15 kDa, 10 kDa to 12 kDa, 15 kDa to 50 kDa, 15 kDa to 45 kDa, 15 kDa to 40 kDa, 15 kDa to 35 kDa, 15 kDa to 30 kDa, 15 kDa to 25 kDa, 15 kDa to 20 kDa, 20 kDa to 50 kDa, 20 kDa to 45 kDa, 20 kDa to 40 kDa, 20 kDa to 35 kDa, 20 kDa to 30 kDa, 20 kDa to 25 kDa, 30 kDa to 50 kDa, 30 kDa to 45 kDa, 30 kDa to 40 kDa, 30 kDa to 35 kDa, or 40 kDa to less than 50 kDa).

Aspect A25: The block copolymer of any one of aspects A1-A24, wherein the block copolymer has an average molecular weight of less than or equal to 40 kDa (e.g., a kDa value of 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0.5).

Aspect A26: The block copolymer of any one of aspects A1-A25, wherein the block copolymer is characterized by a high-density brush block copolymer having a brush density greater than or equal to 90% (e.g., greater than or equal to 92%, greater than or equal to 94%, greater than or equal to 95%, greater than or equal to 96%, greater than or equal to 98%, or greater than or equal to 99%).

Aspect A27: The block copolymer of any one of aspects A1-A26, wherein the block copolymer is characterized by a high-density brush block copolymer having a brush density greater than or equal to 95% (e.g., greater than or equal to 95%, greater than or equal to 96%, greater than or equal to 97%, greater than or equal to 98%, greater than or equal to 99%).

Aspect A28: The block copolymer of any one of aspects A1-A27, wherein the block copolymer is characterized by a high-density brush block copolymer having a brush density greater than or equal to 99% (e.g., greater than or equal to 99%, greater than or equal to 99.1%, greater than or equal to 99.2%, greater than or equal to 99.3%, greater than or equal to 99.4%, greater than or equal to 99.5%, greater than or equal to 99.6%, or greater than or equal to 99.7%, greater than or equal to 99.8%, or greater than or equal to 99.9%).

Aspect A29: The block copolymer of any one of aspects A1-A28, wherein m is an integer having a value of at least 5 (e.g., at least any of the following: 5, 7, 8, 10, 12, 15, 20, 25, or 30, optionally at least any of the following: 35, 40, 45, 50, 55, 60, 65, 70, 75, 80, 85, or 90, optionally, at least any of the following: 100, 150, 200, 250, 300, 350, 400, 450, or 500, optionally less than any of the following: 1000, 950, 900, 850, 800, 750, 700, 650, 600, or 550).

Aspect A30: The block copolymer of any one of aspects A1-A29, wherein P₁ comprises a therapeutic peptide having a chain length of 3 to 100 amino acid residues (e.g., 3 to 50 amino acid residues, 3 to 40 amino acid residues, 3 to 20 amino acid residues, 3 to 10 amino acid residues, 5 to 50 amino acid residues, 5 to 40 amino acid residues, 5 to 20 amino acid residues, 5 to 10 amino acid residues, 10 to 50 amino acid residues, 10 to 40 amino acid residues, 10 to 20 amino acid residues, or 10 to 15 amino acid residues). For example, the therapeutic peptide can comprise 3 to 90, 3 to 80, 3 to 70, 3 to 60, 3 to 50, 3 to 40, 3 to 30, 3 to 20, 3 to 16, 3 to 15, 3 to 14, 4 to 90, 4 to 80, 4 to 70, 4 to 60, 4 to 50, 4 to 40, 4 to 30, 4 to 20, 4 to 16, 4 to 15, 4 to 14, 5 to 90, 5 to 80, 5 to 70, 5 to 60, 5 to 50, 5 to 40, 5 to 30, 5 to 20, 5 to 16, 5 to 15, 5 to 14, 6 to 90, 6 to 80, 6 to 70, 6 to 60, 6 to 50, 6 to 40, 6 to 30, 6 to 20, 6 to 16, 6 to 15, 6 to 14, 7 to 90, 7 to 80, 7 to 70, 7 to 60, 7 to 50, 7 to 40, 7 to 30, 7 to 20, 7 to 16, 7 to 15, 7 to 14, 8 to 100, 8 to 90, 8 to 80, 8 to 70, 8 to 60, 8 to 50, 8 to 40, 8 to 30, 8 to 20, 8 to 16, 8 to 15, 8 to 14, 9 to 100, 9 to 90, 9 to 80, 9 to 70, 9 to 60, 9 to 50, 9 to 40, 9 to 30, 9 to 20, 9 to 16, 9 to 15, 9 to 14, 10 to 90, 10 to 80, 10 to 70, 10 to 60, 10 to 50, 10 to 40, 10 to 30, 10 to 20, 10 to 16, 10 to 15, 10 to 14, 11 to 16, 11 to 15, 11 to 14, 12 to 16, 12 to 15, 12 to 14, 15 to 90, 15 to 80, 15 to 70, 15 to 60, 15 to 50, 15 to 40, 15 to 30, 15 to 20, 15 to 16, 20 to 90, 20 to 80, 20 to 70, 20 to 60, 20 to 50, 20 to 40, or 20 to 30 amino acid units.

Aspect A31: The block copolymer of any one of aspects A1-A30, wherein P₂ comprises a targeting peptide having a chain length of 3 to 100 amino acid residues (e.g., 3 to 50 amino acid residues, 3 to 40 amino acid residues, 3 to 20 amino acid residues, 3 to 10 amino acid residues, 5 to 50 amino acid residues, 5 to 40 amino acid residues, 5 to 20 amino acid residues, 5 to 10 amino acid residues, 10 to 50 amino acid residues, 10 to 40 amino acid residues, 10 to 20 amino acid residues, or 10 to 15 amino acid residues). For example, the targeting peptide can comprise 3 to 90, 3 to 80, 3 to 70, 3 to 60, 3 to 50, 3 to 40, 3 to 30, 3 to 20, 3 to 16, 3 to 15, 3 to 14, 4 to 90, 4 to 80, 4 to 70, 4 to 60, 4 to 50, 4 to 40, 4 to 30, 4 to 20, 4 to 16, 4 to 15, 4 to 14, 5 to 90, 5 to 80, 5 to 70, 5 to 60, 5 to 50, 5 to 40, 5 to 30, 5 to 20, 5 to 16, 5 to 15, 5 to 14, 6 to 90, 6 to 80, 6 to 70, 6 to 60, 6 to 50, 6 to 40, 6 to 30, 6 to 20, 6 to 16, 6 to 15, 6 to 14, 7 to 90, 7 to 80, 7 to 70, 7 to 60, 7 to 50, 7 to 40, 7 to 30, 7 to 20, 7 to 16, 7 to 15, 7 to 14, 8 to 100, 8 to 90, 8 to 80, 8 to 70, 8 to 60, 8 to 50, 8 to 40, 8 to 30, 8 to 20, 8 to 16, 8 to 15, 8 to 14, 9 to 100, 9 to 90, 9 to 80, 9 to 70, 9 to 60, 9 to 50, 9 to 40, 9 to 30, 9 to 20, 9 to 16, 9 to 15, 9 to 14, 10 to 90, 10 to 80, 10 to 70, 10 to 60, 10 to 50, 10 to 40, 10 to 30, 10 to 20, 10 to 16, 10 to 15, 10 to 14, 11 to 16, 11 to 15, 11 to 14, 12 to 16, 12 to 15, 12 to 14, 15 to 90, 15 to 80, 15 to 70, 15 to 60, 15 to 50, 15 to 40, 15 to 30, 15 to 20, 15 to 16, 20 to 90, 20 to 80, 20 to 70, 20 to 60, 20 to 50, 20 to 40, or 20 to 30 amino acid units.

Aspect A32: The block copolymer of any one of aspects A1-A31, wherein P₁ comprises a therapeutic agent and

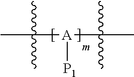

is characterized by a P₁ density of greater than or equal to 90% (e.g., greater than or equal to 92%, greater than or equal to 94%, greater than or equal to 95%, greater than or equal to 96%, greater than or equal to 98%, or greater than or equal to 99%).

Aspect A33: The block copolymer any one of aspects A1-A32, wherein P₁ comprises a therapeutic agent and

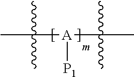

is characterized by a P₁ density of greater than or equal to 95% (e.g., greater than or equal to 95%, greater than or equal to 96%, greater than or equal to 97%, greater than or equal to 98%, greater than or equal to 99%).

Aspect A34: The block copolymer any one of aspects A1-A33, wherein P₁ comprises a therapeutic agent and

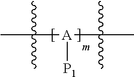

is characterized by a P₁ density of greater than or equal to 99% (e.g., greater than or equal to 99%, greater than or equal to 99.1%, greater than or equal to 99.2%, greater than or equal to 99.3%, greater than or equal to 99.4%, greater than or equal to 99.5%, greater than or equal to 99.6%, or greater than or equal to 99.7%, greater than or equal to 99.8%, or greater than or equal to 99.9%).

Aspect A35: The block copolymer of any one of aspects A1-A34, wherein P₂ comprises a targeting agent and

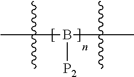

is characterized by a P₂ density of greater than or equal to 90% (e.g., greater than or equal to 92%, greater than or equal to 94%, greater than or equal to 95%, greater than or equal to 96%, greater than or equal to 98%, or greater than or equal to 99%).

Aspect A36: The block copolymer of any one of aspects A1-A35, wherein P₂ comprises a targeting agent and

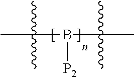

is characterized by a P₂ density of greater than or equal to 95% (e.g., greater than or equal to 95%, greater than or equal to 96%, greater than or equal to 97%, greater than or equal to 98%, greater than or equal to 99%).

Aspect A37: The block copolymer of any one of aspects A1-A36, wherein P₂ comprises a targeting agent and

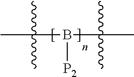

is characterized by a P₂ density of greater than or equal to 99% (e.g., greater than or equal to 99%, greater than or equal to 99.1%, greater than or equal to 99.2%, greater than or equal to 99.3%, greater than or equal to 99.4%, greater than or equal to 99.5%, greater than or equal to 99.6%, or greater than or equal to 99.7%, greater than or equal to 99.8%, or greater than or equal to 99.9%).

Aspect A38: The block copolymer of any one of aspects A1-A37, wherein the block copolymer resists degradation in a subject for at least 3 days. For example, the block copolymer may resist degradation in a subject for 3 or more days, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more days. Alternatively, or in addition, 100 or less days, for example, 90 or less, 80 or less, 70 or less, 60 or less, 59 or less, 58 or less, 57 or less, 56 or less, 55 or less, 54 or less, 53 or less, 52 or less, 51 or less, 50 or less, 49 or less, 48 or less, 47 or less, 46 or less, 45 or less, 44 or less, 43 or less, 42 or less, 41 or less, 40 or less, 39 or less, 38 or less, 37 or less, 36 or less, 35 or less, 34 or less 33 or less, 32 or less, or 31 or less days.

Aspect A39: The block copolymer of any one of aspects A1-A38, wherein at least a portion of the block copolymer is resistant to enzymatic digestion.

Aspect A40: The block copolymer of any one of aspects A1-A39, wherein the block copolymer has a length of less than or equal to 20 nm (e.g., less than or equal to 20 nm, less than to equal to 18 nm, less than or equal to 15 nm, less than or equal to 10 nm, less than or equal to 8 nm, less than or equal to 7 nm, less than or equal to 6 nm, less than or equal to 5 nm, less than or equal to 2 nm, or less than or equal to 1 nm). Alternatively, or in addition, the block copolymer has a length of greater than or equal to 1 nm, greater than or equal to 2 nm, greater than or equal to 3 nm, or greater than or equal to 4 nm.

Aspect A41: The block copolymer of any one of aspects A1-A40, wherein the block copolymer has a length of less than or equal to 15 nm. (e.g., less than or equal to 15 nm, less than or equal to 14 nm, less than or equal to 13 nm, less than or equal to 12 nm, less than or equal to 11 nm, less than or equal to 10 nm, less than or equal to 9 nm, or less than or equal to 8 nm, less than or equal to 7 nm, less than or equal to 6 nm, less than or equal to 5 nm, less than or equal to 2 nm, or less than or equal to 1 nm). Alternatively, or in addition, the block copolymer has a length of greater than or equal to 1 nm, greater than or equal to 2 nm, greater than or equal to 3 nm, or greater than or equal to 4 nm.

Aspect A42: The block copolymer of any one of aspects A1-A41, wherein the block copolymer comprises a formula configured to form crosslinkages with itself.

Aspect A43: The block copolymer of aspect A42, or any preceding aspect, wherein the crosslinkages comprise disulfide bonds.

Aspect A44: The block copolymer of aspect A42 or aspect A43, or any preceding aspect, wherein the formation of crosslinkages with itself results in a block copolymer scaffold.

Aspect A45: The block copolymer of any one of aspects A1-A44, wherein the block copolymer comprises a rigid backbone.

Aspect A46: The block copolymer of any one of aspects A1-A45, wherein the block copolymer comprises an amorphous structure.

Aspect A47. The block copolymer of aspect A46, or any preceding aspect, wherein the amorphous structure facilitates accumulation of the block copolymer at a target site in a subject.

Aspect A48: The block copolymer of aspect A47, or any preceding aspect, wherein the amorphous structure facilitates intracellular penetration of the block copolymer at a target site in a subject.

Aspect A49: The block copolymer of any one of aspects A1-A48, wherein P₂ is a synthetic peptide.

Aspect A50: The block copolymer of any one of aspects A1-A49, wherein P₂ comprises a net cationic sequence.

Aspect A51: The block copolymer of any one of aspects A1-A50, wherein P₂ targets a surface membrane protein.

Aspect A52: The block copolymer of any one of aspects A1-A51, wherein P₂ targets an extracellular receptor or a transmembrane receptor.

Aspect A53: The block copolymer of any one of aspects A1-A52, wherein P₂ comprises a tumor-targeting sequence.

Aspect A54: The block copolymer of any one of aspects A1-A53, wherein P₂ comprises an interleukin-6 inhibitor, an interleukin-10 inhibitor, a JAK/STAT inhibitor, or a LOX inhibitor.

Aspect A55: The block copolymer of any one of aspects A1-A54, wherein P₂ targets an enzyme.

Aspect A56: The block copolymer of aspect A55, or any preceding aspect, wherein P₂ targets an oxidoreductase enzyme or a hydrolase enzyme.

Aspect A57: The block copolymer of aspect A56, or any preceding aspect, wherein P₂ targets an amine oxidase enzyme or a protease enzyme.

Aspect A58: The block copolymer of aspect A57, or any preceding aspect, wherein P₂ targets a lysl oxidase (LOX) enzyme or a matrix metalloproteinase (MMP) enzyme.

Aspect A59: The block copolymer of any one of aspects A51-A58, or any preceding aspect, wherein the term, targets, refers to an interaction with a specific biological molecule.

Aspect A60: The block copolymer of aspect A59, or any preceding aspect, wherein the interaction with a specific biological molecule is an associative interaction.

Aspect A61: The block copolymer of aspect A59 or A60, or any preceding aspect, wherein the specific biological molecule is selected from the group consisting of: a peptide, a polypeptide, a receptor, a surface membrane molecule, an antigen, an enzyme, an ECM protein, an intracellular molecule, or any combination thereof.

Aspect A62: The block copolymer of any one of aspects A59-A61, or any preceding aspect, wherein the specific biological molecule is upregulated in one or more target sites in a subject.

Aspect A63: The block copolymer of aspect A62, or any preceding aspect, wherein the one or more target sites independently comprises an organ, tumor site, an inflammation site, or a tissue,
Aspect A64: The block copolymer of any one of aspects A59-A63, or any preceding aspect, wherein the interaction with a specific biological molecule results in localization of at least a portion of the block copolymer in at least a portion of the one or more target sites.

Aspect A65: The block copolymer of any one of aspects A59-A64, or any preceding aspect, wherein the interaction with a specific biological molecule results in retention of at least a portion of the block copolymer in at least a portion of the one or more target sites.

Aspect A66: The block copolymer of any one of aspects A59-A65, or any preceding aspect, wherein the interaction with a specific biological molecule results in aggregation of at least a portion of the block copolymer with at least a portion of a second block copolymer in at least a portion of the one or more target sites.

Aspect A67: The block copolymer of any one of aspects A1-A66, wherein P₂ comprises a sequence having 75% or greater (e.g., 80% or greater, 85% or greater, 90% or greater, 95% or greater, or 100%) sequence identity of SEQ ID NO: 142 (AAKAAKAA).

Aspect A68: The block copolymer of aspect A67, wherein P₂ comprises a sequence having 85% or greater (e.g., 90% or greater, 95% or greater, or 100%) sequence identity of SEQ ID NO: 142 (AAKAAKAA).

Aspect A69: The block copolymer of aspect A67 or aspect A68, or any preceding aspect, wherein P₂ comprises SEQ ID NO: 142 (AAKAAKAA).

Aspect A70: The block copolymer of aspect A67, wherein the sequence having 75% or greater (e.g., 80% or greater, 85% or greater, 90% or greater, 95% or greater, or 100%) sequence identity of SEQ ID NO: 142 (AAKAAKAA) has a point mutation to comprise an arginine residue.

Aspect A71: The block copolymer of aspect A67 or aspect A70, or any preceding aspect, wherein the sequence having 75% or greater (e.g., 80% or greater, 85% or greater, 90% or greater, 95% or greater, or 100%) sequence identity of SEQ ID NO: 142 (AAKAAKAA) has a point mutation to comprise a glutamate residue.

Aspect A72: The block copolymer of any one of aspects A1-A66, or any preceding aspect, wherein P₂ comprises

SEQ ID NO: 143
(AAKAA);
SEQ ID NO: 144
(AAKAARAA);
SEQ ID NO: 145
(AAKRAA);
SEQ ID NO: 146
(AAKEAA);
SEQ ID NO: 147
(AAEKAA);
SEQ ID NO: 148
(AAEAAKAA);
or
SEQ ID NO: 149
(AAKYAA).

Aspect A73: The block copolymer of any one of aspects A1-A66, or any preceding aspect, wherein P₂ comprises

SEQ ID NO: 184
(AKAK);
SEQ ID NO: 185
(VKVK);
SEQ ID NO: 186
(KKKK);
or
SEQ ID NO: 187
(LKLK)

wherein the third and fourth amino acid residues of each sequence identity are optionally repeated between 0 to 49 times. For example, the third and fourth amino acid residues of each sequence identity may be repeated between 0 to 49, 0 to 40, 0 to 30, 0 to 20, 0 to 15, 0 to 14, 0 to 12, 0 to 10, 0 to 8, 0 to 6, 0 to 5, 0 to 4, 0 to 3, 0 to 2, 0 to 1, 1 to 49, 1 to 40, 1 to 30, 1 to 20, 1 to 15, to 14, to 12, 1 to 10, 1 to 8, 1 to 6, 1 to 5, 1 to 4, 1 to 3, 1 to 2, 2 to 49, 2 to 40, 2 to 30, 2 to 20, 2 to 15, 2 to 14, 2 to 12, 2 to 10, 2 to 8, 2 to 6, 2 to 5, 2 to 4, 2 to 3, 3 to 49, 3 to 40, 3 to 30, 3 to 20, 3 to 15, 3 to 14, 3 to 12, 3 to 10, 3 to 8, 3 to 6, 3 to 5, 3 to 4, 4 to 49, 4 to 40, 4 to 30, 4 to 20, 4 to 15, 4 to 14, 4 to 12, 4 to 10, 4 to 8, 4 to 6, 4 to 5, 5 to 49, 5 to 40, 5 to 30, 5 to 20, 5 to 15, to 14, 5 to 12, 5 to 10, 5 to 8, 5 to 6, 6 to 49, 6 to 40, 6 to 30, 6 to 20, 6 to 15, 6 to 14, 6 to 12, 6 to 10, 6 to 8, 10 to 49, 10 to 40, 10 to 30, 10 to 20, 10 to 15, 10 to 14, or 10 to 12 times.

Aspect A74: The block copolymer of any one of aspects A1-A66, or any preceding aspect, wherein P₂ comprises a sequence having 75% or greater (e.g., 80% or greater, 85% or greater, 90% or greater, 95% or greater, or 100%) sequence identity of SEQ ID NO: 150 (GGGGEKGGGGG).

Aspect A75: The block copolymer of aspect A74, or any preceding aspect, wherein P₂ comprises a sequence having 85% or greater (e.g., 90% or greater, 95% or greater, or 100%) sequence identity of SEQ ID NO: 150 (GGGGEKGGGGG).

Aspect A76: The block copolymer of aspect A74 or aspect A75, or any preceding aspect, wherein P₂ comprises SEQ ID NO: 150 (GGGGEKGGGGG).

Aspect A77: The block copolymer of aspect A74, or any preceding aspect, wherein the sequence having 75% or greater (e.g., 80% or greater, 85% or greater, 90% or greater, 95% or greater, or 100%) sequence identity of SEQ ID NO: 150 (GGGGEKGGGGG) has a point mutation to comprise a glutamine residue.

Aspect A78: The block copolymer of any one of aspects A1-A66, or any preceding aspect, wherein P₂ comprises

SEQ ID NO: 151
(GGGGGKGGGGG);
SEQ ID NO: 152
(GGGGGKEGGGG);
SEQ ID NO: 153
(GGGGQKGGGGG);
SEQ ID NO: 189
(GGGDQKGGGGG);
SEQ ID NO: 190
(GGGDPKGGGGG);
or
SEQ ID NO: 191
(GGGQEKGGGGG).

Aspect A79: The block copolymer of any one of aspects A1-A66, or any preceding aspect, wherein P₂ comprises

(SEQ ID NO 217 and 218)
(ΦPGG)n-V
(SEQ ID NO 219 and 220)
(VPGΦG)n-V;
or
(SEQ ID NO 221 and 222);
V(APGΦGV)n

wherein
each Φ independently comprises V or K at approximately a 4:1 ratio;
the first amino acid residue is optionally attached to a carbonyl group;
the last amino acid residue is optionally attached to a methoxy group; and
each n is an integer having a value greater than or equal to 40 (e.g., 40 or greater, 45 or greater, 50 or greater, 55 or greater, 60 or greater, 65 or greater, 70 or greater, 75 or greater, 80 or greater, 85 or greater, 90 or greater, or 95 or greater.) Alternatively, or in addition, n is an integer having a value less than or equal to 200, less than or equal to 150, less than or equal to 125, or less than or equal to 100.

Aspect A80: The block copolymer of aspect A79, or any preceding aspect, wherein P₂ comprises

SEQ ID NO: 217
(VPGGV);
SEQ ID NO: 218
(KPGGV);
SEQ ID NO: 219
(VPGVGV);
SEQ ID NO: 220
(VPGKGV);
SEQ ID NO: 221
(VAPGVGV);
or
SEQ ID NO: 222
(VAPGKGV).

Aspect A81: The block copolymer of any one of aspects A1-A66, or any preceding aspect, wherein P₂ comprises

SEQ ID NO: 195
(ELSYGYDEKSTG);
SEQ ID NO: 196
(GYDEKST);
SEQ ID NO: 197
(GYDEKSA);
SEQ ID NO: 198
(GFDEKAG);
SEQ ID NO: 199
(GYDEKAG);
SEQ ID NO: 200
(AYDVKSG);
SEQ ID NO: 201
(SYDVKSG);
SEQ ID NO: 202
(FDAKGG);
SEQ ID NO: 203
(QYDGKGV);
SEQ ID NO: 204
(QYDPSKA);
SEQ ID NO: 205
(YSDKGV);
SEQ ID NO: 206
(PQQEKAH);
SEQ ID NO: 207
(GQREKGP);
SEQ ID NO: 208
(IGAEKAG);
SEQ ID NO: 209
(GAGEKGP);
or
SEQ ID NO: 210
(VGGEKSG).

Aspect A82: The block copolymer of any one of aspects A1-A66, or any preceding aspect, wherein P₂ comprises a sequence having 75% or greater (e.g., 80% or greater, 85% or greater, 90% or greater, 95% or greater, or 100%) sequence identity of SEQ ID NO: 213 (AKGS), wherein the first amino acid residue of the sequence identity is optionally attached to an N-acetyl group.

Aspect A83: The block copolymer of any one of aspects A1-A82, wherein P₂ comprises a sequence having a length selected from the range of 4.8 Å to 14.2 Å (e.g., 4.8 Å to 14.2 Å, 4.9 Å to 14.2 Å, 5.0 Å to 14.2 Å, 5.1 Å to 14.2 Å, 5.2 Å to 14.2 Å, 5.3 Å to 14.2 Å, 5.4 Å to 14.2 Å, 5.5 Å to 14.2 Å, 5.6 Å to 14.2 Å, 5.7 Å to 14.2 Å, 5.8 Å to 14.2 Å, 5.9 Å to 14.2 Å, 6.0 Å to 14.2 Å, 6.1 Å to 14.2 Å, 6.2 Å to 14.2 Å, 6.3 Å to 14.2 Å, 6.4 Å to 14.2 Å, 6.5 Å to 14.2 Å, 6.6 Å to 14.2 Å, 6.7 Å to 14.2 Å, 6.8 Å to 14.2 Å, 6.9 Å to 14.2 Å, 7.0 Å to 14.2 Å, 7.1 Å to 14.2 Å, 7.2 Å to 14.2 Å, 7.3 Å to 14.2 Å, 7.4 Å to 14.2 Å, 7.5 Å to 14.2 Å, 7.6 Å to 14.2 Å, 7.7 Å to 14.2 Å, 7.8 Å to 14.2 Å, 7.9 Å to 14.2 Å, 8.0 Å to 14.2 Å, 8.1 Å to 14.2 Å, 8.2 Å to 14.2 Å, 8.3 Å to 14.2 Å, 8.4 Å to 14.2 Å, 8.5 Å to 14.2 Å, 8.6 Å to 14.2 Å, 8.7 Å to 14.2 Å, 8.8 Å to 14.2 Å, 8.9 Å to 14.2 Å, 9.0 Å to 14.2 Å, 9.1 Å to 14.2 Å, 9.2 Å to 14.2 Å, 9.3 Å to 14.2 Å, 9.4 Å to 14.2 Å, 9.5 Å to 14.2 Å, 9.6 Å to 14.2 Å, 9.7 Å to 14.2 Å, 9.8 Å to 14.2 Å, 9.9 Å to 14.2 Å, 10.0 Å to 14.2 Å, 10.1 Å to 14.2 Å, 10.2 Å to 14.2 Å, 10.3 Å to 14.2 Å, 10.4 Å to 14.2 Å, 10.5 Å to 14.2 Å, 10.6 Å to 14.2 Å, 10.7 Å to 14.2 Å, 10.8 Å to 14.2 Å, 10.9 Å to 14.2 Å, 11.0 Å to 14.2 Å, 11.1 Å to 14.2 Å, 11.2 Å to 14.2 Å, 11.3 Å to 14.2 Å, 11.4 Å to 14.2 Å, 11.5 Å to 14.2 Å, 11.6 Å to 14.2 Å, 11.7 Å to 14.2 Å, 11.8 Å to 14.2 Å, 11.9 Å to 14.2 Å, 12.0 Å to 14.2 Å, 12.1 Å to 14.2 Å, 12.2 Å to 14.2 Å, 12.3 to 14.2 Å, 12.4 to 14.2 Å, 12.5 to 14.2 Å, 12.6 to 14.2 Å, 12.7 to 14.2 Å, 12.8 Å to 14.2 Å, 12.9 Å to 14.2 Å, 13.0 Å to 14.2 Å, 13.1 Å to 14.2 Å, 13.2 Å to 14.2 Å, 13.3 to 14.2, 13.4 to 14.2, 13.5 to 14.2 Å, 13.6 to 14.2 Å, 13.7 to 14.2 Å, 13.8 to 14.2 Å, 13.9 Å to 14.2 Å, 14.0 Å to 14.2 Å, or 14.1 Å to 14.2 Å).

Aspect A84: The block copolymer of any one of aspects A1-A83, wherein P₂ comprises a sequence having at least one lysine residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 lysine residues). Alternatively, or in addition, P₂ comprises a sequence having less than 150, 125, or 100 lysine residues.

Aspect A85: The block copolymer of any one of aspects A1-A66, or any preceding aspect, wherein P₂ comprises a sequence having at least one methylated lysine residue. (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 methylated lysine residues). Alternatively, or in addition, P₂ comprises a sequence having less than 150, 125, or 100 methylated lysine residues.

Aspect A86: The block copolymer of any one of aspects A1-A66, or any preceding aspect, wherein P₂ comprises a sequence having 75% or greater (e.g., 80% or greater, 85% or greater, 90% or greater, 95% or greater, or 100%) sequence identity of SEQ ID NO: 154 (GPLGLAGGWGERDGS).

Aspect A87: The block copolymer of aspect A86, or any preceding aspect, wherein P₂ comprises a sequence having 85% or greater (e.g., 90% or greater, 95% or greater, or 100%) sequence identity of SEQ ID NO: 154 (GPLGLAGGWGERDGS).

Aspect A88: The block copolymer of aspect A86 or aspect A87, or any preceding aspect, wherein P₂ comprises SEQ ID NO: 154 (GPLGLAGGWGERDGS).

Aspect A89: The block copolymer of any one of aspects A1-A66, or any preceding aspect, wherein P₂ comprises a sequence having 75% or greater (e.g., 80% or greater, 85% or greater, 90% or greater, 95% or greater, or 100%) sequence identity of SEQ ID NO: 155 (GGSGSGSGWGERDGS).

Aspect A90: The block copolymer of aspect A89, or any preceding aspect, wherein P₂ comprises a sequence having 85% or greater (e.g., 90% or greater, 95% or greater, or 100%) sequence identity of SEQ ID NO: 155 (GGSGSGSGWGERDGS).

Aspect A91: The block copolymer of aspect A89 or aspect A90, or any preceding aspect, wherein P₂ comprises SEQ ID NO: 155 (GGSGSGSGWGERDGS).

Aspect A92: The block copolymer of any one of aspects A1-A66, or any preceding aspect, wherein P₂ comprises a sequence having 75% or greater (e.g., 80% or greater, 85% or greater, 90% or greater, 95% or greater, or 100%) sequence identity of SEQ ID NO: 214 (CSTSMLKAC).

Aspect A93: The block copolymer of any one of aspects A1-A66, or any preceding aspect, wherein P₂ comprises a sequence having 75% or greater (e.g., 80% or greater, 85% or greater, 90% or greater, 95% or greater, or 100%) sequence identity of SEQ ID NO: 215 (CKPGTSSYC).

Aspect A94: The block copolymer of any one of aspects A1-A66, or any preceding aspect, wherein P₂ comprises a sequence having 75% or greater (e.g., 80% or greater, 85% or greater, 90% or greater, 95% or greater, or 100%) sequence identity of, or SEQ ID NO: 216 (CPDRSVNNC).

Aspect A95: The block copolymer of any one of aspects A1-A94, wherein each P₁ independently treats or manages a condition of a subject.

Aspect A96: The block copolymer of any one of aspect A1-A95, wherein each P₁ independently comprises a synthetic peptide.

Aspect A97: The block copolymer of any one of aspects A1-A96, wherein each P₁ independently comprises an anti-inflammatory peptide, an anti-microbial peptide, a wound healing peptide, a myocardial infarction treatment peptide, a cancer treatment peptide, or any combination thereof.

Aspect A98: The block copolymer of any one of aspects A1-A96, or any preceding aspect, wherein each P₁ independently treats or manages a neurodegenerative disease, inflammation, an inflammatory disease, myocardial infarction, cancer, a musculoskeletal disease, or any combination thereof.

Aspect A99: The block copolymer of any one of aspects A1-A98, wherein P₁ comprises a sequence having 75% or greater (e.g., 80% or greater, 85% or greater, 90% or greater, 95% or greater, or 100%) sequence identity of SEQ ID NO: 1 (LDEETGEFL).

Aspect A100: The block copolymer of aspect A99, or any preceding aspect, wherein P₁ comprises a sequence having 85% or greater (e.g., 90% or greater, 95% or greater, or 100%) sequence identity of SEQ ID NO: 1 (LDEETGEFL).

Aspect A101: The block copolymer of aspect A99 or aspect A100, or any preceding aspect, wherein P₁ comprises SEQ ID NO: 1 (LDEETGEFL).

Aspect A102: The block copolymer of aspect A99, or any preceding aspect, wherein the sequence having 75% or greater (e.g., 80% or greater, 85% or greater, 90% or greater, 95% or greater, or 100%) sequence identity of SEQ ID NO: 1 (LDEETGEFL) has a point mutation to comprise a proline residue.

Aspect A103: The block copolymer of aspect A102, or any preceding aspect, wherein P₁ comprises SEQ ID NO: 2 (LDPETGEFL).

Aspect A104: The block copolymer of aspect A99 or aspect A102, or any preceding aspect, wherein the sequence having 75% or greater (e.g., 80% or greater, 85% or greater, 90% or greater, 95% or greater, or 100%) sequence identity of SEQ ID NO: 1 (LDEETGEFL) has a point mutation to delete a glutamate residue.

Aspect A105: The block copolymer of aspect A104, or any preceding aspect, wherein P₁ comprises SEQ ID NO: 137 (LDPTGEFL) or SEQ ID NO: 138 (LDPETGFL).

Aspect A106: The block copolymer of any one of aspects A1-A98, or any preceding aspect, wherein P₁ is

SEQ ID NO: 3
(LDEETGEFLRR);
SEQ ID NO: 4
(LDEETGEFLRRR);
SEQ ID NO: 5
(LDEETGEFLRRRR);
SEQ ID NO: 6
(LDEETGEFLRRRRR);
SEQ ID NO: 7
(RRLDEETGEFL);
SEQ ID NO: 8
(RRRLDEETGEFL);
SEQ ID NO: 9
(RRRRLDEETGEFL);
SEQ ID NO: 10
(RRRRRLDEETGEFL);
SEQ ID NO: 11
(RRLDEETGEFLRR);
SEQ ID NO: 12
(RRRLDEETGEFLRRR);
SEQ ID NO: 13
(RLDEETGEFLR);
SEQ ID NO: 14
(RLDEETGEFLRR);
SEQ ID NO: 15
(RRLDEETGEFLR);
SEQ ID NO: 16
(RLDEETGEFLRRR);
SEQ ID NO: 17
(RRRLDEETGEFLR);
SEQ ID NO: 18
(RLDEETGEFLRRRR);
SEQ ID NO: 19
(RRLDEETGEFLRRR);
SEQ ID NO: 20
(RRRLDEETGEFLRR);
SEQ ID NO: 21
(RRRRLDEETGEFLR);
SEQ ID NO: 22
(RLDEETGEFLRRRRR);
SEQ ID NO: 23
(RRLDEETGEFLRRRR);
SEQ ID NO: 24
(RRRRLDEETGEFLRR);
SEQ ID NO: 25
(RRRRRLDEETGEFLR);
SEQ ID NO: 26
(LDEETGEFLKK);
SEQ ID NO: 27
(LDEETGEFLKKK);
SEQ ID NO: 28
(LDEETGEFLKKKK);
SEQ ID NO: 2
(LDEETGEFLKKKKK);
SEQ ID NO: 30
(KKLDEETGEFL);
SEQ ID NO: 31
(KKKLDEETGEFL);
SEQ ID NO: 32
(KKKKLDEETGEFL);
SEQ ID NO: 33
(KKKKKLDEETGEFL);
SEQ ID NO: 34
(KKLDEETGEFLKK);
SEQ ID NO: 35
(KKKLDEETGEFLKKK);
SEQ ID NO: 36
(KLDEETGEFLK);
SEQ ID NO: 37
(KLDEETGEFLKK);
SEQ ID NO: 38
(KKLDEETGEFLK);
SEQ ID NO: 39
(KLDEETGEFLKKK);
SEQ ID NO: 40
(KKKLDEETGEFLK);
SEQ ID NO: 41
(KLDEETGEFLKKKK);
SEQ ID NO: 42
(KKLDEETGEFLKKK);
SEQ ID NO: 43
(KKKLDEETGEFLKK);
SEQ ID NO: 44
(KKKKLDEETGEFLK);
SEQ ID NO: 45
(KLDEETGEFLKKKKK);
SEQ ID NO: 46
(KKLDEETGEFLKKKK);
SEQ ID NO: 47
(KKKKLDEETGEFLKK);
SEQ ID NO: 48
(KKKKKLDEETGEFLK);
SEQ ID NO: 49
(LDEETGEFLKRKR);
SEQ ID NO: 50
(KRKRLDEETGEFL);
SEQ ID NO: 51
(RKRKLDEETGEFL);
SEQ ID NO: 52
(LDEETGEFLRKRK);
SEQ ID NO: 53
(KKLDEETGEFLRR);
SEQ ID NO: 54
(RRLDEETGEFLKK);
SEQ ID NO: 55
(KLDEETGEFLRRR);
SEQ ID NO: 56
(KKKLDEETGEFLR);
SEQ ID NO: 57
(RRRLDEETGEFLK);
SEQ ID NO: 58
(KRLDEETGEFLKR);
SEQ ID NO: 59
(RKLDEETGEFLRK);
SEQ ID NO: 60
(RKLDEETGEFLKR);
SEQ ID NO: 61
(KRLDEETGEFLRK);
SEQ ID NO: 62
(LDEETGEFLKKRR);
SEQ ID NO: 63
(LDEETGEFLRRKK);
SEQ ID NO: 64
(KKRRLDEETGEFL);
SEQ ID NO: 65
(RRKKLDEETGEFL);
SEQ ID NO: 66
(LDEETGEFLGSGSGRR);
SEQ ID NO: 67
(GSGSGRRLDEETGEFL);
SEQ ID NO: 68
(LDEETGEFLGSGSGKK);
SEQ ID NO: 69
(GSGSGKKLDEETGEFL);
SEQ ID NO: 70
(LDPETGEFLRR);
SEQ ID NO: 71
(LDPETGEFLRRR);
SEQ ID NO: 72
(LDPETGEFLRRRR);
SEQ ID NO: 73
(LDPETGEFLRRRRR);
SEQ ID NO: 74
(RRLDPETGEFL);
SEQ ID NO: 75
(RRRLDPETGEFL);
SEQ ID NO: 76
(RRRRLDPETGEFL);
SEQ ID NO: 77
(RRRRRLDPETGEFL);
SEQ ID NO: 78
(RRLDPETGEFLRR);
SEQ ID NO: 79
(RRRLDPETGEFLRRR);
SEQ ID NO: 80
(RLDPETGEFLR);
SEQ ID NO: 81
(RLDPETGEFLRR);
SEQ ID NO: 82
(RRLDPETGEFLR);
SEQ ID NO: 83
(RLDPETGEFLRRR);
SEQ ID NO: 84
(RRRLDPETGEFLR);
SEQ ID NO: 85
(RLDPETGEFLRRRR);
SEQ ID NO: 86
(RRLDPETGEFLRRR);
SEQ ID NO: 87
(RRRLDPETGEFLRR);
SEQ ID NO: 88
(RRRRLDPETGEFLR);
SEQ ID NO: 89
(RLDPETGEFLRRRRR);
SEQ ID NO: 90
(RRLDPETGEFLRRRR);
SEQ ID NO: 91
(RRRRLDPETGEFLRR);
SEQ ID NO: 92
(RRRRRLDPETGEFLR);
SEQ ID NO: 93
(LDPETGEFLKK);
SEQ ID NO: 94
(LDPETGEFLKKK);
SEQ ID NO: 95
(LDPETGEFLKKKK);
SEQ ID NO: 96
(LDPETGEFLKKKKK);
SEQ ID NO: 97
(KKLDPETGEFL);
SEQ ID NO: 98
(KKKLDPETGEFL);
SEQ ID NO: 99
(KKKKLDPETGEFL);
SEQ ID NO: 100
(KKKKKLDPETGEFL);
SEQ ID NO: 101
(KKLDPETGEFLKK);
SEQ ID NO: 102
(KKKLDPETGEFLKKK);
SEQ ID NO: 103
(KLDPETGEFLK);
SEQ ID NO: 104
(KLDPETGEFLKK);
SEQ ID NO: 105
(KKLDPETGEFLK);
SEQ ID NO: 106
(KLDPETGEFLKKK);
SEQ ID NO: 107
(KKKLDPETGEFLK);
SEQ ID NO: 108
(KLDPETGEFLKKKK);
SEQ ID NO: 109
(KKLDPETGEFLKKK);
SEQ ID NO: 110
(KKKLDPETGEFLKK);
SEQ ID NO: 111
(KKKKLDPETGEFLK);
SEQ ID NO: 112
(KLDPETGEFLKKKKK);
SEQ ID NO: 113
(KKLDPETGEFLKKKK);
SEQ ID NO: 114
(KKKKLDPETGEFLKK);
SEQ ID NO: 115
(KKKKKLDPETGEFLK);
SEQ ID NO: 116
(LDPETGEFLKRKR);
SEQ ID NO: 117
(KRKRLDPETGEFL);
SEQ ID NO: 118
(RKRKLDPETGEFL);
SEQ ID NO: 119
(LDPETGEFLRKRK);
SEQ ID NO: 120
(KKLDPETGEFLRR);
SEQ ID NO: 121
(RRLDPETGEFLKK);
SEQ ID NO: 122
(KLDPETGEFLRRR);
SEQ ID NO: 123
(KKKLDPETGEFLR);
SEQ ID NO: 124
(RRRLDPETGEFLK);
SEQ ID NO: 125
(KRLDPETGEFLKR);
SEQ ID NO: 126
(RKLDPETGEFLRK);
SEQ ID NO: 127
(RKLDPETGEFLKR);
SEQ ID NO: 128
(KRLDPETGEFLRK);
SEQ ID NO: 129
(LDPETGEFLKKRR);
SEQ ID NO: 130
(LDPETGEFLRRKK);
SEQ ID NO: 131
(KKRRLDPETGEFL);
SEQ ID NO: 132
(RRKKLDPETGEFL);
SEQ ID NO: 133
(LDPETGEFLGSGSGRR);
SEQ ID NO: 134
(GSGSGRRLDPETGEFL);
SEQ ID NO: 135
(LDPETGEFLGSGSGKK);
SEQ ID NO: 136
(GSGSGKKLDPETGEFL);
SEQ ID NO: 140
(YGRKKRRLDPETGEFL);
or
SEQ ID NO: 141
(LDPETGEFLYGRKKRR).

Aspect A107: The block copolymer of any one of aspects A1-A98, or any preceding aspect, wherein at least one, and optionally all, of P₁ comprises a compound having the formula of C₁₇H₁₈BrNO₄S. For example, 100%, 99% or more, 98% or more, 97% or more, 96% or more, 95% or more, 90% or more, 85% or more, 80% or more, 75% or more, 70% or more, 65% or more, 60% or more, 55% or more, or 50% or more of P₁ comprises a compound having the formula of C₁₇H₁₈BrNO₄S. Alternatively, or in addition, at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least thirty-five, at least forty, at least forty-five, at least fifty, at least one-hundred, at least one-hundred and fifty, at least two-hundred, at least two-hundred and fifty, or at least five-hundred of P₁ comprises a compound having the formula of C₁₇H₁₈BrNO₄S.

Aspect A108: The block copolymer of any one of aspects A1-A98, or any preceding aspect, wherein at least one, and optionally all, of P₁ comprises a compound having the formula of C₂₂H₂₄N₂O₈. For example, 100%, 99% or more, 98% or more, 97% or more, 96% or more, 95% or more, 90% or more, 85% or more, 80% or more, 75% or more, 70% or more, 65% or more, 60% or more, 55% or more, or 50% or more of P₁ comprises a compound having the formula of C₂₂H₂₄N₂O₈. Alternatively, or in addition, at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least thirty-five, at least forty, at least forty-five, at least fifty, at least one-hundred, at least one-hundred and fifty, at least two-hundred, at least two-hundred and fifty, or at least five-hundred of P₁ comprises a compound having the formula of C₂₂H₂₄N₂O₈.

Aspect A109: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 156 (ISYGNDALMP).

Aspect A110: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 157 (SLINGPAYMD).

Aspect A111: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of TIMP-1.

Aspect A112: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 158 (GACLRSGRGCG).

Aspect A113: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 159 (GAALRSGRGAG).

Aspect A114: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 160 (PRCCGE), wherein a biphenylalanine (B) is optionally present between the third and fourth amino acid residues of the sequence having 75% or greater sequence identity of SEQ ID NO: 160. For example, P₁ may comprise PRC(B)CGE.

Aspect A115: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 161 (MCMPCFTTDHQMARKCDDCCGGKGRGKCYGPQCLCR).

Aspect A116: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 162 (HWWQWPSSLQLRGGGS).

Aspect A117: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 163 (HNWTRWLLHPDRGGGS).

Aspect A118: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 164 (GACFSIAHECGA).

Aspect A119: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 165 (VMDGYPMP).

Aspect A120: The block copolymer of aspect A119, or any preceding aspect, wherein the sequence having 75% or greater (e.g., 80% or greater, 85% or greater, 90% or greater, 95% or greater, or 100%) sequence identity of SEQ ID NO: 165 (VMDGYPMP) comprises an acetyl group attached to the N-terminus of the sequence identity and an amino group attached to the C-terminus of the sequence identity.

Aspect A121: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 166 (GYPKSALR).

Aspect A122: The block copolymer of aspect A121, or any preceding aspect, wherein the sequence having 75% or greater (e.g., 80% or greater, 85% or greater, 90% or greater, 95% or greater, or 100%) sequence identity of SEQ ID NO: 166 (GYPKSALR) comprises an acetyl group attached to the N-terminus of the sequence identity and an amino group attached to the C-terminus of the sequence identity.

Aspect A123: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 167 (NENLLRFFVAPFPEV).

Aspect A124: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 168 (CRVYGPYLLC).

Aspect A125: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 169 (ADGACGYGRFSPPCGAAG).

Aspect A126: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 170 (ADGACILWMDDGWCGAAG).

Aspect A127: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 171 (STTHWGFTLC).

Aspect A128: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 172 (SLRRSSCFGGRMDRIGAQSGLGCNSFRY).

Aspect A129: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 173 (SLRRSSCFGGRIDRIGAQSGLGCNSFRY).

Aspect A130: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 174 (MGSFSITLGFFLVLAFWLPGHIGPNPVYSAVSNTD).

Aspect A131: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 175 (SQGSTLRVQQRPQNSKVTHISSCFGHKIDRIGSVSRLGCNALKLL).

Aspect A132: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 176 (NSKMAHSSSCFGQKIDRIGAVSRLGCDGLRLF).

Aspect A133: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 177 (SPKMVQGSGCFGRKMDRISSSSGLGCKVLRRH).

Aspect A134: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 178 (GLSKGCFGLKLDRIGSMSGLGC).

Aspect A135: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 179 (EVKYDPCFGHKIDRINHVSNLGCPSLRDPRPNAPSTSA).

Aspect A136: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 180 (MAKSGIYLGCFTLILIQNMVA).

Aspect A137: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 181 (AMVSEFLKQAWFIENEEQEYVQTVK).

Aspect A138: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 182 (TAPRSLRRSSCFGGRMDRIGAQSGLGCNSFRY).

Aspect A139: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 183 (DNKPPKKGPPNGCFGHKIDRIGSHSGLGCNKVDDNKG).

Aspect A140: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 225 (RPKPQQFFGLM).

Aspect A141: The block copolymer of any one of aspects A1-A98, or any preceding aspect, wherein P₁ comprises a sequence having 75% or greater sequence identity of SEQ ID NO: 223 (GYGSSSRRAPQT).

Aspect A142: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of Annexin-A1 N-Terminal Peptide.

Aspect A143: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of a VEGF peptide.

Aspect A144: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of a ghrelin peptide.

Aspect A145: The block copolymer of any one of aspects A1-A98, or any preceding aspect, wherein P₁ comprises a sequence having 75% or greater (e.g., 80% or greater, 85% or greater, 90% or greater, 95% or greater, or 100%) sequence identity of SEQ ID NO: 224 (CTTHWGFTLC).

Aspect A146: The block copolymer of any one of aspects A1-A145, wherein P₁ further comprises a charge modulating domain.

Aspect A147: The block copolymer of aspect A146, or any preceding aspect, wherein the charge modulating domain has from 2 to 7 amino acid residues (e.g., from 2 to 6 amino acid residues, from 2 to 5 amino acid residues, from 2 to 4 amino acid residues, from 2 to 3 amino acid residues, from 3 to 7 amino acid residues, from 4 to 7 amino acid residues, from 5 to 7 amino acid residues, or from 6 to 7 amino acid residues). The 2 to 7 amino acids can be added in a single block containing from 2 to 7 amino acid residues or more than one block containing from 1 to 6 amino acid residues.

Aspect A148: The block copolymer of aspect A146 or aspect A147, or any preceding aspect, wherein the charge modulating domain is a glycine-serine domain, a cationic residue domain, or a combination thereof.

Aspect A149: The block copolymer of any one of aspects A1-A148, wherein P₁ has from 11 to 16 amino acid residues (e.g., 11 amino acid residues, 12 amino acid residues, 13 amino acid residues, 14 amino acid residues, 15 amino acid residues, or 16 amino acid residues).

Aspect A150: The block copolymer of any one of aspects A1-A149, wherein Q₃ is a one or more backbone monomers, such as substitute or unsubstituted norbornene (e.g., as norbornene imide, norbornene amide, etc), or one or more methylacrylamide or acrylamide backbone monomers.

Aspect A151: The block copolymer of any one of aspects A1-A149, wherein Q₃ is a single bond or —O—.

Aspect A151: The block copolymer of any one of aspects A1-A149, wherein Q₃ is a substituted or unsubstituted C₁-C₂₀ alkylene, C₂-C₂₀ heteroalkylene, C₃-C₂₀ cycloalkylene, C₃-C₂₀ arylene, C₃-C₂₀ heteroarylene, C₂-C₂₀ alkenylene, C₂-C₂₀ cycloalkenylene, C₁-C₂₀ alkoxy, or C₁-C₂₀ acyl, optionally a substituted or unsubstituted C₁-C₁₀ alkylene, C₂-C₁₀ heteroalkylene, C₃-C₁₀ cycloalkylene, C₃-C₁₀ arylene, C₃-C₁₀ heteroarylene, C₂-C₁₀ alkenylene, C₂-C₁₀ cycloalkenylene, C₁-C₁₀ alkoxy, or C₁-C₁₀ acyl.

Aspect B1: A pharmaceutical composition comprising the block copolymer of any one of aspects A1-A148 and a pharmaceutically acceptable excipient.

Aspect B2: The pharmaceutical composition of aspect B1 comprising the block copolymer of any one of aspects A107-A149.

Aspect C1: A method of treating or managing a condition of a subject comprising:
administering to the subject a therapeutically effective amount of the block copolymer of any one of aspects A1-A149 or the pharmaceutical composition of aspect B1, or aspect B2;
wherein the administering results in the treating or managing of a condition of the subject.

Aspect C2: The method of aspect C1 further comprising:
repeating the step of administering to the subject the therapeutically effective amount of the block copolymer of any one of aspects A1-A149 or the pharmaceutical composition of aspect B1, or aspect B2.

Aspect C3: The method of aspect C1 or C2, wherein the method results in an accumulation of P₁ at a disease site in the subject.

Aspect C4: The method of any one of aspects C1-C3, wherein the administering to the subject comprises intravenous administration, subcutaneous administration, intramuscular administration, topical administration, oral administration, or a combination thereof.

Aspect C5: The method of any one of aspects C1-C4, wherein the condition is associated with an inflammatory state, increased oxidative stress, autoimmune pathophysiology, chemo-preventative measures, neurodegeneration, or a combination thereof.

Aspect C6: The method of any one of aspects C1-C5, wherein the condition is an autoimmune disease.

Aspect C7: The method of aspect C6, or any preceding aspect, wherein the autoimmune disease is multiple sclerosis, systemic lupus erythematous, Sjogren syndrome, rheumatoid arthritis, vitiligo, or psoriasis.

Aspect C8: The method of any one of aspects C1-C5, or any preceding aspect, wherein the condition is a respiratory disease.

Aspect C9: The method of aspect C8, or any preceding aspect, wherein the respiratory disease is COPD, emphysema, potential treatment for smokers, idiopathic pulmonary fibrosis, chronic sarcoidosis, or hypersensitivity pneumonitis.

Aspect C10: The method of any one of aspects C1-C5, or any preceding aspect, wherein the condition is a gastrointestinal disease.

Aspect C11: The method of aspect C10, or any preceding aspect wherein the gastrointestinal disease is ulcerative colitis, ulcers, prevent acetaminophen toxicity, non-alcoholic steatohepatitis, primary biliary cholangitis, cirrhosis, type 2 diabetes, or diabetic nephropathy.

Aspect C12: The method of any one of aspects C1-C5, or any preceding aspect, wherein the condition is a cardiovascular disease.

Aspect C13: The method of aspect C12, or any preceding aspect, wherein the cardiovascular disease is myocardial infarction, cardiac ischemia-reperfusion injury, heart failure, or atherosclerosis.

Aspect C14: The method of any one of aspects C1-C5, or any preceding aspect, wherein the condition is a neurodegenerative disease.

Aspect C15: The method of aspect C14, or any preceding aspect, wherein the neurodegenerative disease is Alzheimer's disease, Parkinson's disease, amyotrophic lateral sclerosis (ALS), Huntington's disease, Friedreich ataxia, or frontotemporal lobar degeneration.

Aspect C16: The method of any one of aspects C1-C5, or any preceding aspect, wherein the method interrupts the protein-protein interaction between Nuclear factor (erythroid-derived 2)-like 2 (Nrf2) and Kelch-like ECH-Associating protein 1 (Keap1).

Aspect C17: The method of any one of aspects C1-C5, or any preceding aspect, wherein the method inhibits activity of MMP enzymes and/or LOX enzymes.

### EXAMPLES

In the following examples, the block copolymer of the present invention and related formulations are occasionally referred to as protein-like polymers (PLPs). It will be understood by a skilled artisan that these terms maybe be used interchangeably subject to the surrounding context and that the block copolymer of the present invention is a member of the PLP class.

Materials: All amino acids used to prepare peptides by solid-phase peptide synthesis (SPPS) were obtained from AAPPTec, Chem-Impex, and NovaBiochem. All reagents were obtained from chemical sources and used without further purification. Polymerizations were performed in a dry nitrogen atmosphere using dimethylformamide (DMF) as the solvent. For kinetics experiments, sealed ampules of DMF-d7 (Sigma Aldrich) were used. N-(hexanoic acid)-cis-5-norbornene-exo-dicarboximide and the olefin metathesis initiator (IMesH2)(C5H5N)2(C1)₂Ru=CHPh were synthesized according to standard procedures. Cy5.5 dye and NorCy5.5 were synthesized according to standard procedures. Enzymes α-Chymotrypsin and Thermolysin were purchased from Sigma Aldrich. Phosphate Buffered Saline (DPBS) without Ca2+ and Mg2+ was purchased from Corning Cellgro.

### Instrumentation:

¹H Nuclear Magnetic Resonance (¹H NMR): ¹H NMR data was recorded on either a Bruker Avance III HD 500 MHz spectrometer, a Bruker Avance III 600 MHz spectrometer, or a Bruker Neo 600 MHz spectrometer. The 500 MHz spectrometer was primarily used for small molecule characterization, the Bruker Avance III 600 MHz spectrometer was used for polymerization kinetics studies, and the Bruker Neo 600 MHz spectrometer was used for diffusion ordered spectroscopy (DOSY) studies done on PLPs in deuterated buffered saline. ¹H NMR chemical shifts are given in reference to residual proton peaks from either the DMSO-d6 or the DMF-d7 solvent.

Analytical High-Performance Liquid Chromatography (HPLC): Analytical HPLC analysis of peptides, peptide monomers, and degradation kinetics were performed on a Jupiter 4μ Proteo90 Å Phenomenex column (150×4.60 mm) using a Hitachi-Elite LaChrom L-2130 pump equipped with UV-Vis detector (Hitachi-Elite LaChromL2420) monitoring at 214 nm. The solvent system consists of (A) 0.1% TFA in water and (B) 0.1% TFA in acetonitrile.

Preparative HPLC: Synthesized peptides, peptide monomers, and both the Cy5.5 dye and NorCy5.5 monomer were purified on a preparative-scale Jupiter Proteo90 Å Phenomenex column (25×250 mm) with an Armen Spot Prep II System. All RP-HPLC was performed with a gradient buffer system in which Buffer A is 0.1% TFA in water and Buffer B is 0.1% TFA in acetonitrile.

Electrospray Ionization Mass Spectrometry (ESI-MS): ESI-MS spectra of peptides were collected using a Bruker Amazon-SL spectrometer configured with an ESI source in both negative and positive ionization modes.

Organic Phase Gel Permeation Chromatography (Organic GPC): Polymer molecular weights (Mn), degrees of polymerization (DP) and dispersities (Mw/Mn) were determined by size-exclusion chromatography coupled with multiangle light scattering (SEC-MALS). SEC was performed with a Phenomenex Phenogel 5μ, 1K-75K (300×7.8 mm) column in series with a Phenomenex Phenogel 5μ, 10K-1000K (300×7.8 mm) column, which was run in 0.05 M LiBr in dimethylformamide (DMF) using a flow rate of 0.75 mL/min controlled by a Shimadzu pump. Also in sequence were a Hitachi UV-Vis Detector L-2420, a Wyatt DAWN®HELEOS® II multiangle light scattering detector operating at 659 nm, and a Wyatt Optilab T-rEX refractive index (RI) detector operating at 659 nm. This SEC-MALS system was normalized to a 30K polystyrene standard.

LI-COR Odyssey Clx Infrared Fluorescent Western Scanning/Imaging System (LI-COR Odyssey): For whole organ and segmented organs scanning and quantification, organs were kept on ice until scanning. A clear transparency was placed on the scanner and the organs were arranged and scanned at intensity level 1 at the 700-nanometer wavelength with an offset of 1 millimeter. LI-COR scanning occurred with row by row scans that were sequentially added to a larger image containing all organs. Scanned images were analyzed using a custom MATLAB script where individual organs, the left ventricle, and right ventricle were outlined and analyzed for Cy5.5 fluorescence intensity per area.

Abcam Fluorometric Activity Assay: To determine LOX activity, an Abcam fluorometric assay was used. In the presence of a proprietary substrate from Abcam, if a given tissue has LOX enzyme present, hydrogen peroxide is released. A secondary reaction with horseradish peroxidase and HRP fluorescent substrate, which emits light that can be read around 570-590 nm, is then measured. Samples are read in triplicate per condition. Typical plate readers (Cytek, Tecan) can be used to measure fluorescence.

Confocal Microscopy: Confocal microscopy is used with spectra ranging from blue (400 nm) to far red (680 nm). Samples are cryosectioned (10 uM sections) onto positively charged slides to allow for the sample to stick to the slide. For immunohistochemistry, samples are fixed in 4% paraformaldehyde for up to 5 minutes, and subjected to blocking buffer. Primary and secondary antibodies are added to visualize targets of interest. DAPI is added to visualize nuclei. Slides are coverslipped with fluoromount and left to dry overnight. For confocal imaging, typical instruments include Nikon Ti2 Eclipse, Zeiss 780, and Andor Dragonfly. Samples are cleaned with ethanol prior to imaging. For images at 40× or above, oil is added to the objective to visualize the sample and specific targets of interest.

alamarBlue: To determine overall cell viability and metabolic profile, alamarBlue is a cell permeating dye that measures the ATP levels of living cells. After 24 hours of co-culture with PLPs, alamarBlue is added at a 1:10 ratio (for every 100 uL, 10 uL of alamarBlue is added). After 4 hours, a plate reader is used to measure the absorbance (detected at 570 and 600 nm) or fluorescence (using an excitation between 530-560 and an emission at 590 nm). Typical plate readers (Cytek, Tecan) can be used to measure fluorescence or absorbance. Cells can be given fresh media after reading cell health, and alamarBlue can be used for time course studies on cell viability.

### Example 1

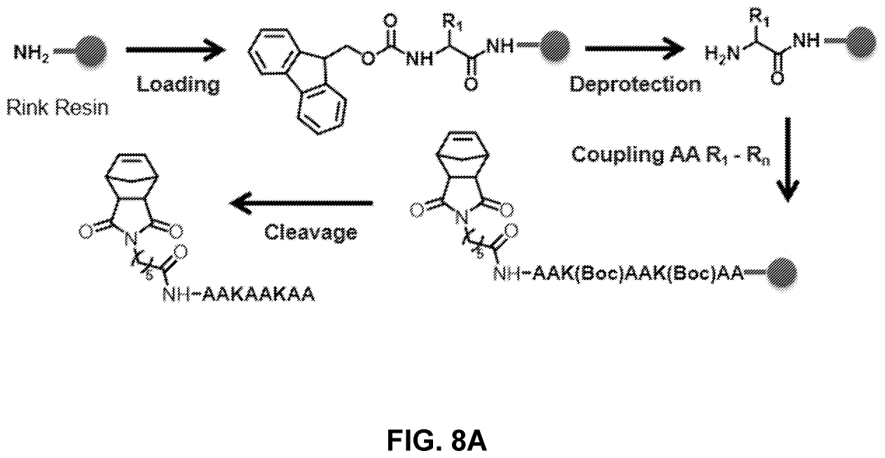

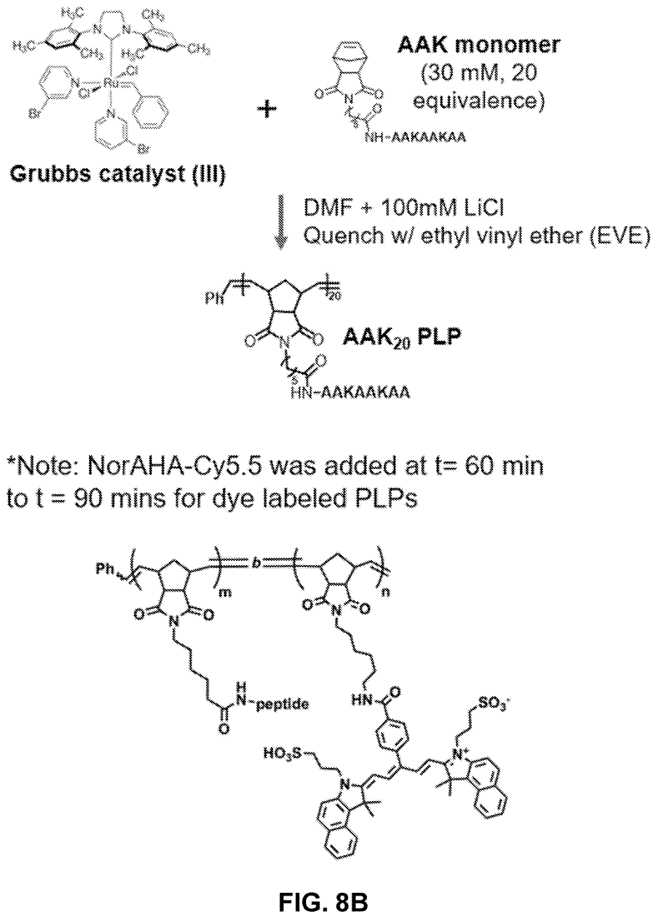

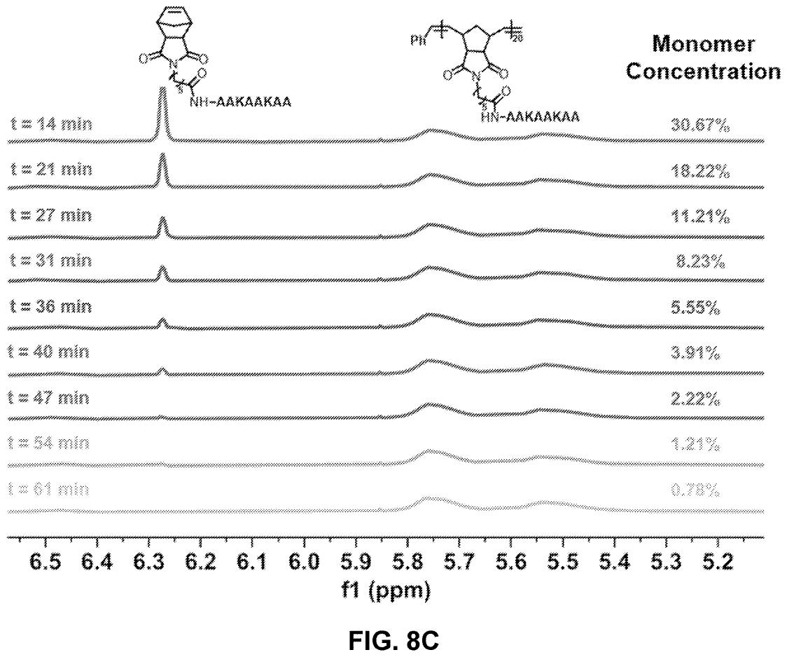

This example provides an exemplary synthesis of a polynorbornene dicarboxyimide-based block copolymer comprising an exemplary targeting agent SEQ ID NO: 142 (AAKAAKAA), and a non-therapeutic agent with an optionally bound fluorescent dye, as depicted in FIGS. 8A and 8B.

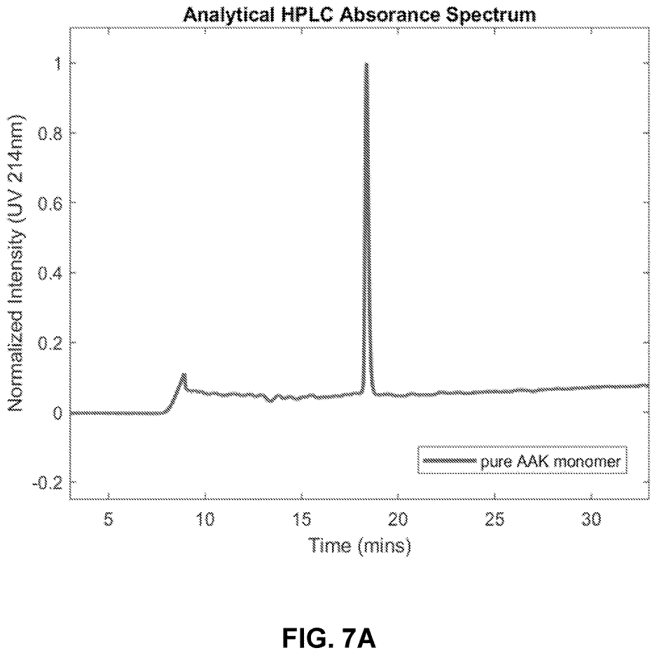

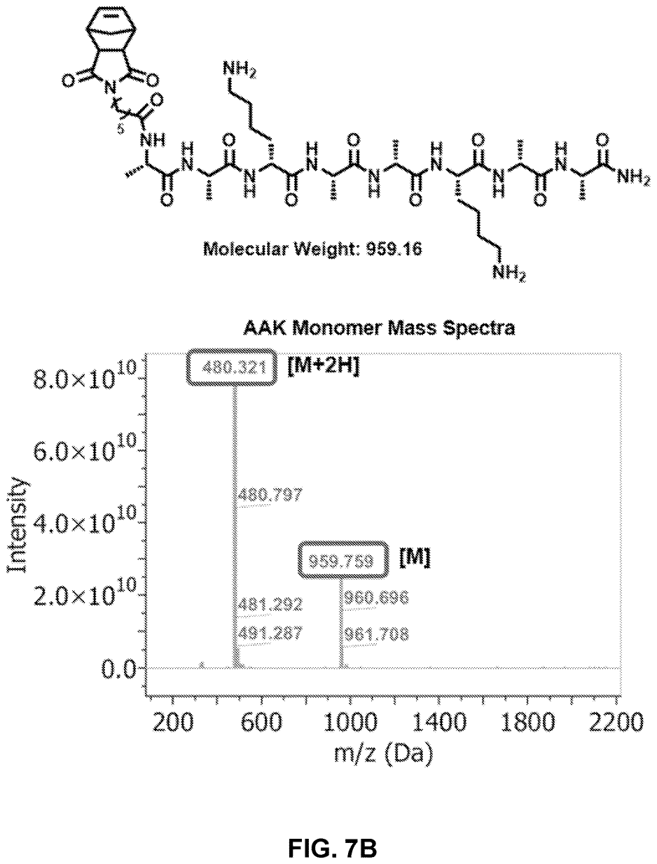

Synthesis Methods: A peptide monomer with SEQ ID NO: 142 (AAKAAKAA) (i.e., “AAK monomer”) was synthesized on rink resin with a Liberty Blue Microwave Peptide Synthesizer through solid-phase peptide synthesis as shown in FIG. 8A. AAK monomer was cleaved from the resin using a TFA/TIPS/H₂O (95:2.5:2.5) cleavage cocktail, precipitated into ether, and dried with N₂ gas. AAK monomer was solubilized in water and purified by Preparative High Performance Liquid Chromatography (HPLC) using a gradient of 0-12-38% buffer B (Acetonitrile+0.01% TFA) over 0-5-30 minutes (buffer A: water+0.01% TFA) (FIG. 7A). Mass characterization was performed on pure AAK monomer by electron spray ionization mass spectroscopy (MS-ESI) (FIG. 7B).

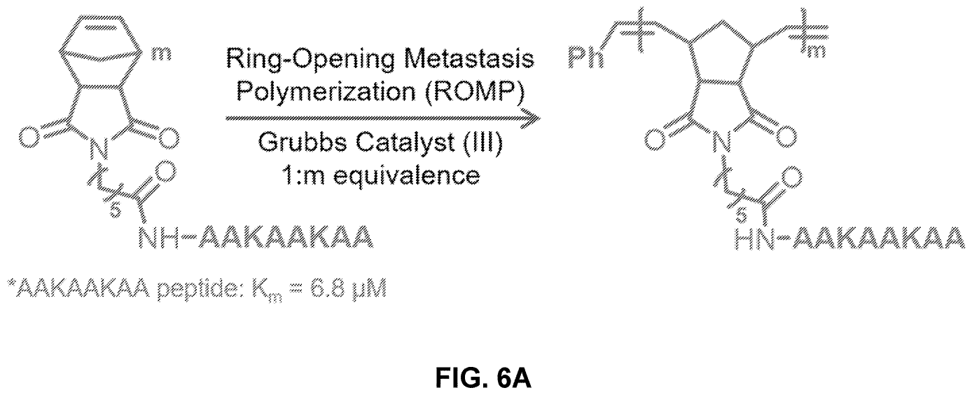

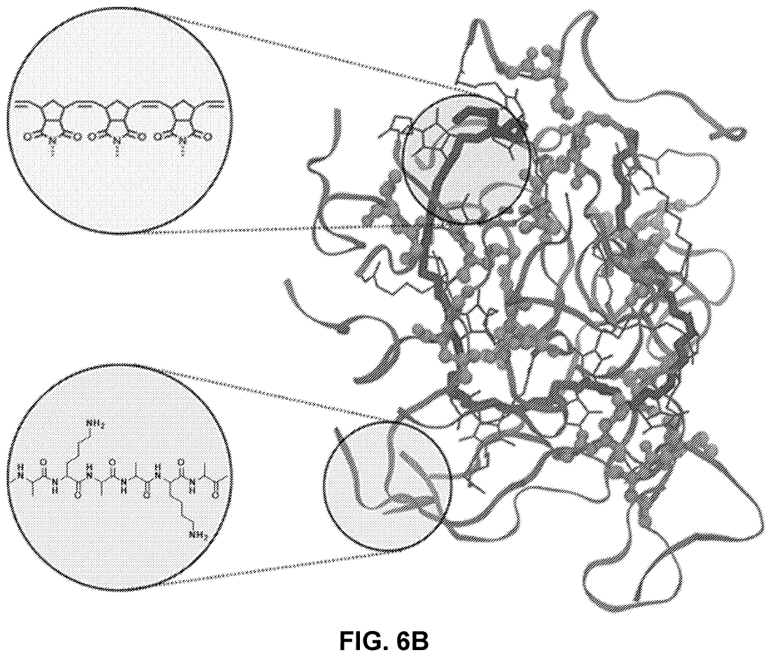

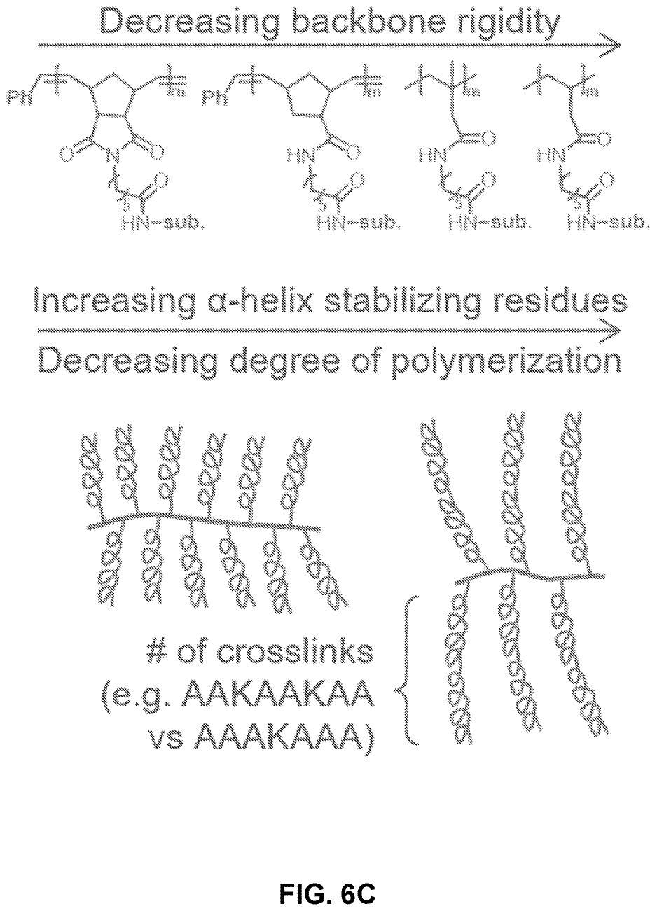

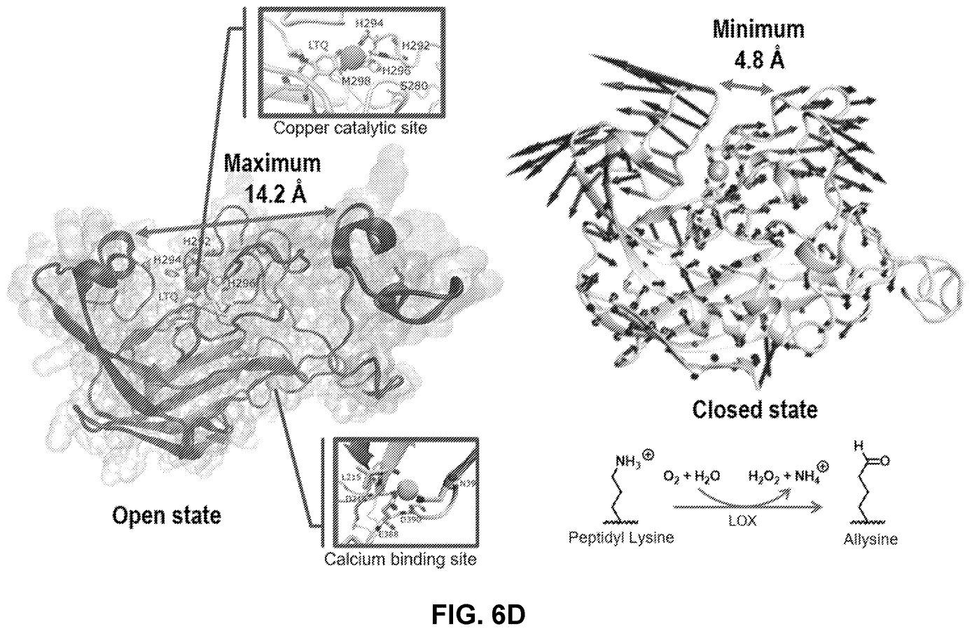

AAK₂₀ PLPs (or “AAK₂₀”) were synthesized through ring-opening metastasis polymerization (ROMP) using 30 mM AAK monomer at 20 molar equivalence to Grubbs catalyst (III) as shown in FIG. 6A and FIG. 8B. The kinetics of this reaction was monitored by Proton Nuclear Magnetic Resonance (NMR) and found to polymerize to completion in 60 minutes (FIG. 8C). In AAK₂₀ PLP samples to be labeled with a fluorophore (i.e., Cy5.5-AAK₂₀), as shown at the bottom of FIG. 8B, a NorAHA-cy5.5 monomer was added at 1 molar equivalence to the catalyst at t=60 minutes and was consumed by t=90 minutes. In both unlabeled and labeled AAK₂₀ PLP reactions, ethyl vinyl ether (EVE) was used to quench the reaction at t=60 minutes and t=90 minutes, respectively. These samples were dialyzed into water and lyophilized into dry powders.

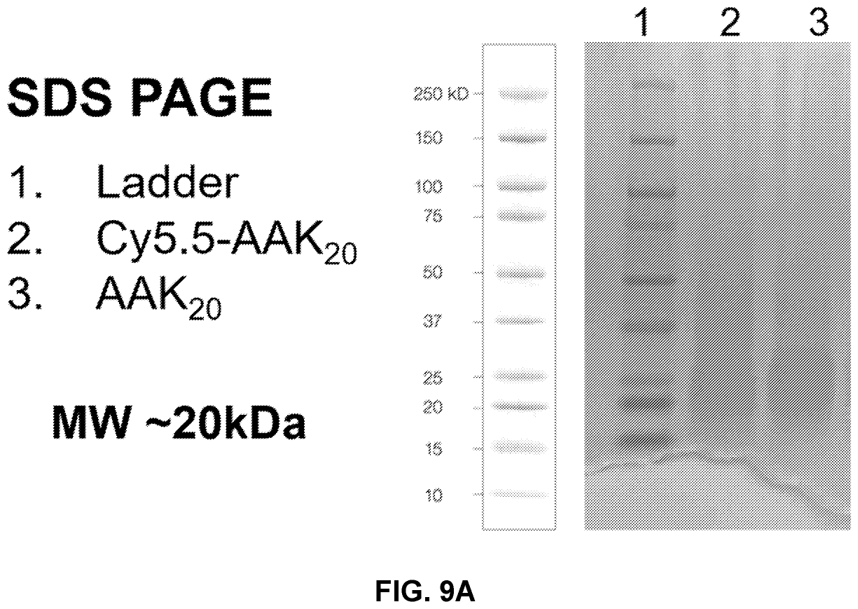

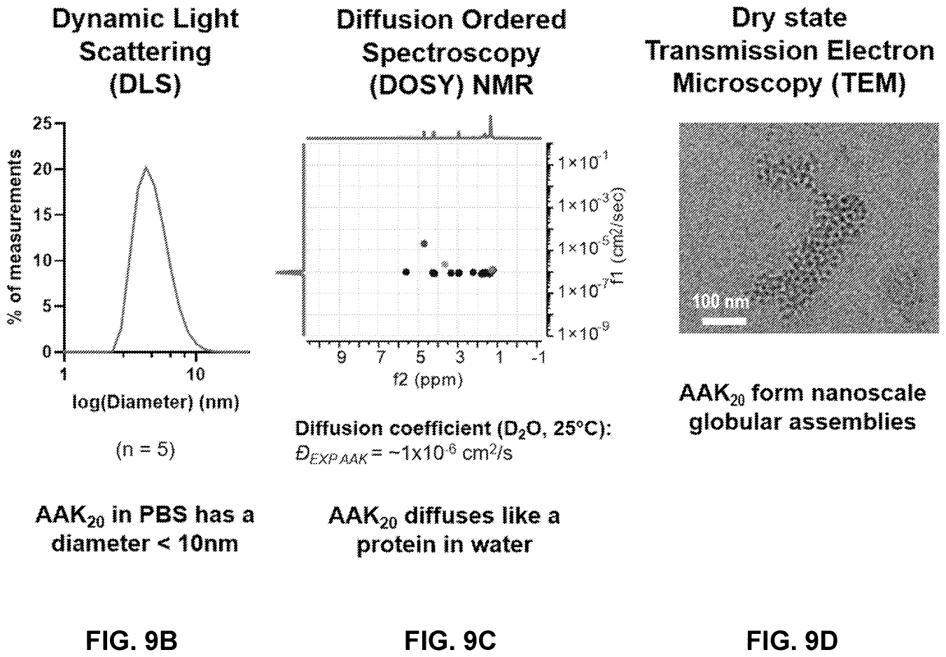

Characterization Results: The molar mass of both unlabeled and labeled AAK₂₀ PLPs were analyzed by SDS PAGE using a polyacrylamide gel with a pore gradient of 4-15% (cat #: 4561083) and Precision Plus Kaleidoscope Protein Standard (cat #: 1610395). The mass of both AAK₂₀ and Cy5.5-AAK₂₀ samples were ˜20 kDa and found to correspond with the average molecular weight of an AAK PLP with a DP of 20 (FIG. 9A). The average diameter of AAK₂₀ in PBS was analyzed by Dynamic Light Scattering (DLS) and was found to be <10 nm, as expected for a PLP (FIG. 9B). The diffusion rate of AAK₂₀ in deuterated water was analyzed by Diffusion Ordered Spectroscopy (DOSY) NMR. AAK₂₀ was found to diffuse at a rate of approximately 1×10⁻⁶ cm²/s, as depicted in FIG. 9C, which is on the order of protein diffusion in water. Additional physical characterization of AAK₂₀ by dry state Transmission Electron Microscopy (TEM) was performed to provide complementary data to SDS PAGE, DLS, and DOSY NMR. AAK₂₀ was found to form nanoscale globular assemblies as shown in FIG. 9D.

The composition of the block copolymer, having one block more hydrophilic than the other block, results in an amorphous structure that generally does not form large micellar structures. Indeed, the disclosed formulations typically comprise structures having diameters of ˜2-3 nanometers in size. Additionally, PLPs in general have a molecular weight of ˜20-40 kDa. Without wishing to be bound by any particular theory, it is believed that the morphology, smaller size, and lower molecular weight compared to previously published nanoparticles allows the block copolymer to preferentially accumulate in the kidneys over the liver, where passage through the glomerular basement membrane requires a hydrodynamic radius of less than 6 nm. Additionally, morphology also plays a role in clearance and accumulation. Because of their amorphous, protein-like structure, the block copolymers described herein have a longer half-life in circulation and a higher aspect ratio, allowing for easier diffusion through the glomerular filtration barrier. Longer circulation times may also contribute to the significant degree of accumulation observed in the heart. Finally, the block copolymer retention in the kidneys allows for excretion through urine, a clearance pathway completing a process which accounts for a majority of drug excretion and is much simpler than hepatic clearance. This is further confirmed by the fact that the filtration-size threshold for globular proteins is <5 nm in diameter.

The low molecular weight and amphiphilicity of the block copolymer also contributes to the block copolymer's ability to penetrate intracellular sites of a subject. Therapeutic agents alone are generally composed of hydrophobic material, which may result in premature aggregation in solution due to the lack of a hydrophilic region that a targeting agent bonded to the same backbone imparts. As such, block copolymer amphiphilicity plays a role in favorable accumulation and biodistribution in a target site.

### Example 2

The following methods and results demonstrate characteristics (retention, localization, cytocompatibility, etc.) of an exemplary block copolymer PLP in a target site, namely a heart. The representative polymer of these examples includes a targeting agent configured to target an infarcted myocardium, in part, by interacting with upregulated lysyl oxidase (LOX) known in the art to be present in a reperfused heart. A representative depiction of the LOX enzyme is provided in FIG. 6B.

Methods 2.1: Confirmation of Acute Myocardial Infarction (MI) LOX Presence in Rat Ischemia-Reperfusion (IR) MI Model. MI was induced in rats via IR and harvested at the following timepoints: 6 hours, 12 hours, 1 day, and 3 days, with healthy hearts as a control (n=3). RT-qPCR was performed for LOX and GAPDH as a reference gene at 6 hours, 12 hours, 1 day, and 3 days post-MI (n=3). Additionally, an Abcam Fluorometric LOX Activity Assay was performed at 1-day and 3-days post-MI with healthy hearts as controls (n=3). Finally, validation of activity and gene expression data was performed with immunohistochemistry for LOX through representative images of healthy myocardium, 1-day post-MI tissue, and 3-day post-MI tissue.

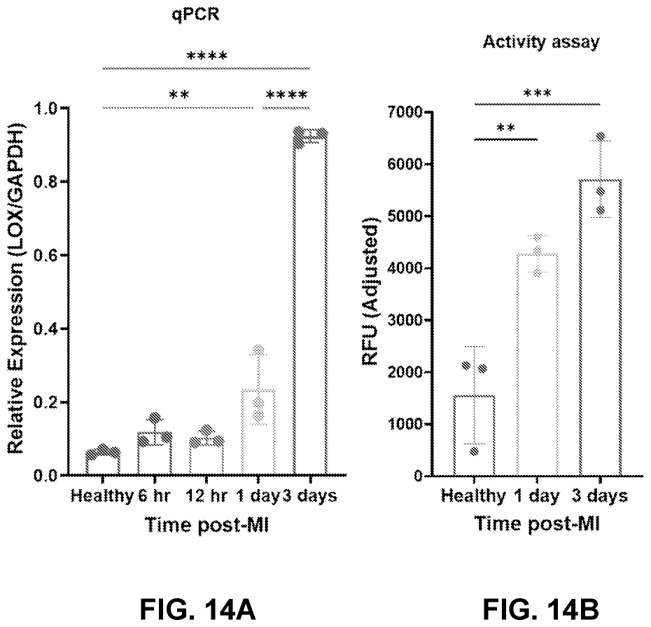

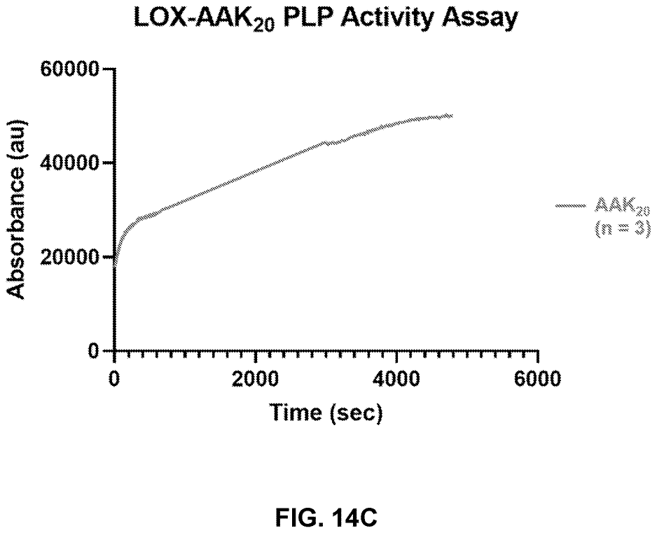

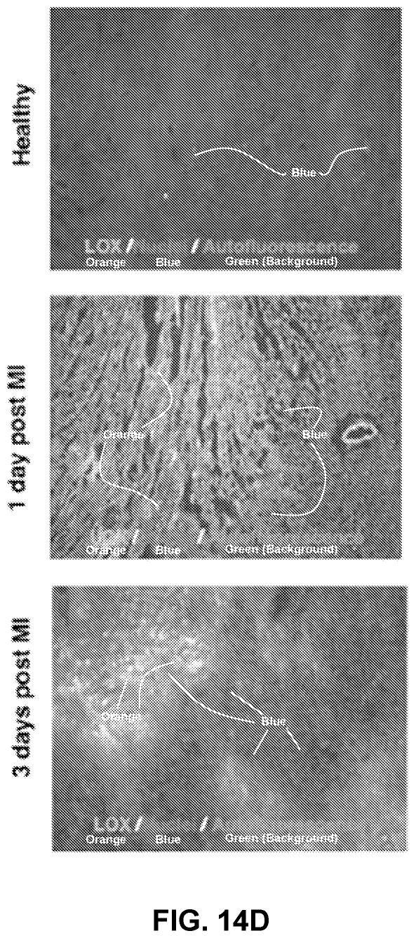

Results 2.1: Increased relative LOX gene expression over time was observed in MI as shown in FIG. 14A with a significant increase of expression at 1- and 3-days post-MI compared to healthy hearts control. Similarly, FIG. 14B exhibits an observed increase in LOX activity at 1- to 3-days post-MI compared to healthy hearts control shown in FIG. 14C. FIG. 14D compliments the results shown in FIGS. 14A and 14B by displaying the observed increased LOX staining in infarct at 1- and 3-days post-MI compared to healthy hearts control (FIG. 14C). These results support the conclusion of an increased presence of LOX post-MI at acute timepoints. Previously, the earliest timepoint LOX was known to be expressed was 3 days post-MI. With the present results showing expression upregulation at acute timepoints, LOX's crosslinking capabilities are exploitable to target and deliver a therapeutic to the infarct. As such, LOX is a targetable biomarker capable of assembling a scaffold of LOX-responsive substrates in the infarct upon minimally invasive administration.

Methods 2.2: Evaluation of LOX-Responsive PLP Systemic Retention in the Infarcted Myocardium. A pilot study using a rat IR MI model was employed to evaluate whether a LOX-responsive PLP would crosslink in the infarct and to itself in the presence of LOX enzyme. The LOX-responsive PLP was created using the synthesis method described in Example 1 with a polynorbornene backbone and a targeting agent comprising a LOX-responsive targeting agent (AAKAAKAA). The PLP was labeled with Cy5.5 to visualize localization. 300 μM of the Cy5.5-labeled LOX-responsive PLPs was administered intravenously through tail-vein injection at 1 day post-MI (n=2) based on the preliminary LOX target validation at 1-day post-MI that found higher relative LOX activity at that timepoint and the clinical relevance of administering at very acute timepoints (FIG. 14B). Animals were harvested 2 days post injection (3 days post-MI). Overall localization was measured via LI-COR Odyssey on transverse sectioned hearts at 700 nm. The LI-COR data was validated and LOX-responsive PLPs were visualized within the injured myocardium via anti-alpha-actinin and anti-LOX antibody immunohistochemistry staining within the infarct, showing infarct targeting of the LOX-responsive PLPs.

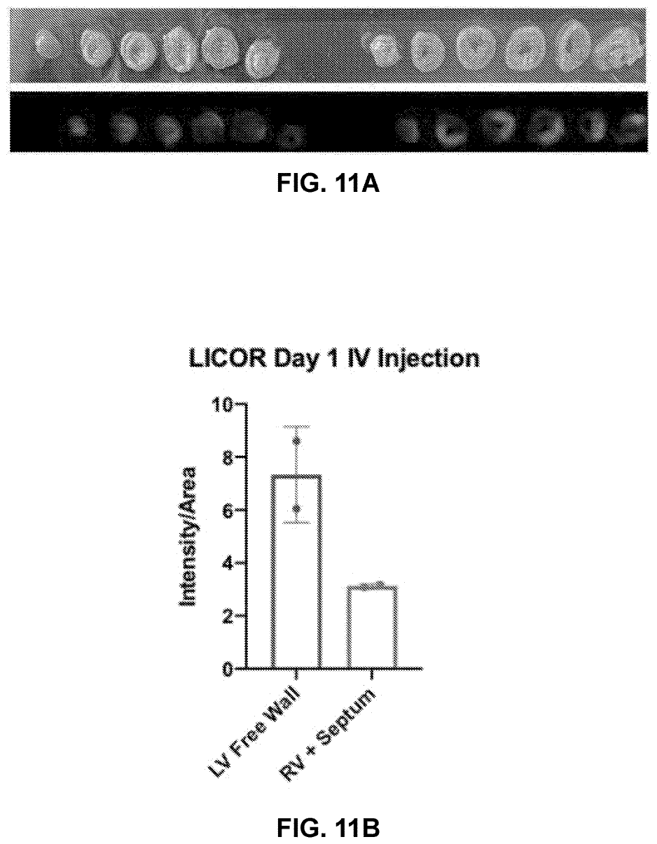

Results 2.2: LOX-responsive PLP retention was observed in the sectioned hearts, as depicted in FIG. 11A, where the fluorescent red coloring in the bottom photo of FIG. 11A portrays the presence of Cy5.5-labeled LOX-responsive PLP. The LI-COR measurements were used to compare fluorescence in the left ventricle (LV) free wall vs. the right ventricle (RV) and septum. Based on these measurements, there was a trending higher amount of retention in the infarcted LV compared to the RV and septum (FIG. 11B).

Methods 2.3: In vivo Evaluation of LOX-Responsive PLP Systemic Retention in the Infarcted Myocardium. To validate in vivo accumulation in the infarct, an acute timepoint study was performed using a rat IR model. Healthy rats were used as the control group. Rats (N=2) were surgically given an MI at t=0 and were treated with 300 μM cy5.5-AAK₂₀ via tail vein (TV) injection at t=6 hours, 12 hours, 1 day, 2 days, 3 days, 7 days (N=1), and 28 days. Cy5.5-AAK₂₀ was allowed to circulate for 2 days post-injection. The animal was then sacked, and the heart and the satellite organs (lungs, liver, kidneys, and spleen) were harvested. The heart was sliced along the short axis into 6 segments. The slices were stained with α-actinin for cardiomyocytes, DAPI for cell nuclei, and anti-LOX antibody (cat #: ab31238) for LOX and imaged via bright field fluorescence microscopy for cy5.5-AAK₂₀.

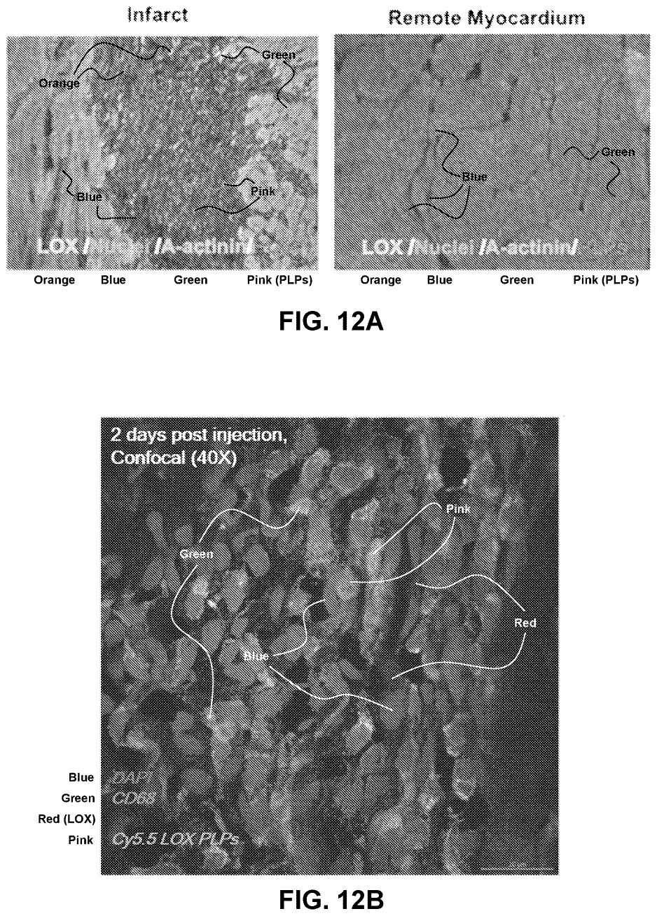

Results 2.3: A representative image of cy5.5-AAK₂₀ localization to the infarct of the animal injected 2 days post-MI is shown in FIG. 12A and can be compared to the remote myocardium in the same figure. As demonstrated in the key of FIG. 12A, LOX presence is indicated by orange, and cy5.5-AAK₂₀ is indicated by pink. Trace amounts, if any, of LOX and cy5.5-AAK₂₀ were observed in the remote myocardium, which supports AAK₂₀ localization ability. Confocal microscopy was used to resolve colocalization of cy5.5-AAK₂₀ with CD68 macrophages, stained with anti-CD68 antibody (FIG. 12B). The colocalization of cy5.5-AAK₂₀ with CD68 in the infarct demonstrates AAK₂₀ potential ability to target a site in a subject comprising an elevated concentration of macrophages. This colocalization also suggests macrophage mediated clearance of the AAK₂₀ via remodeling processes.

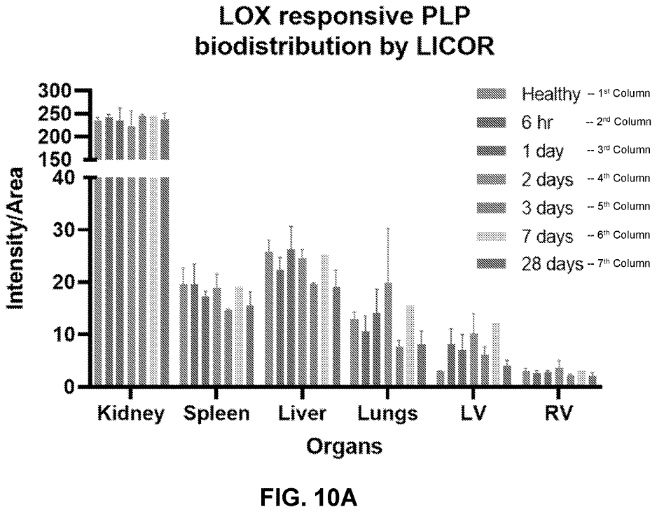

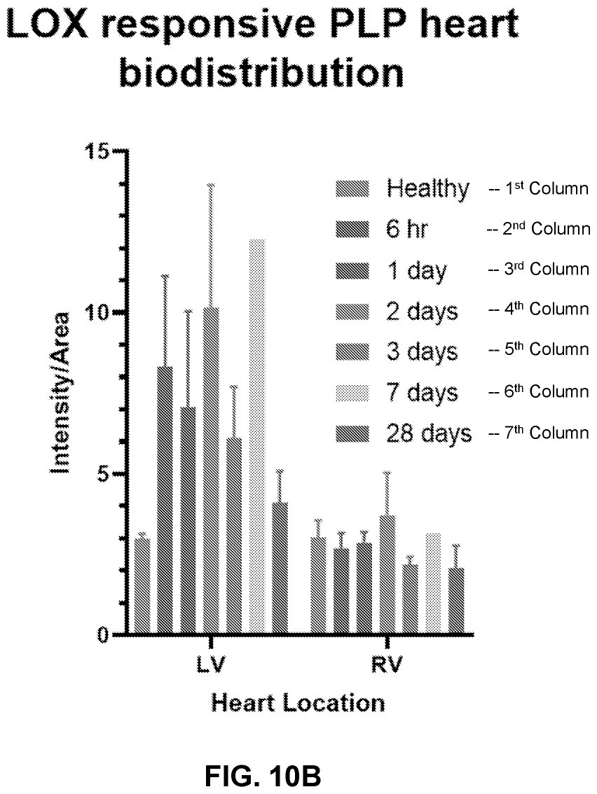

Additionally, LI-COR imaging was used to quantify biodistribution of cy5.5-AAK₂₀ across the heart and satellite organs. FIG. 10A shows said biodistribution during acute timepoints in the heart and satellite organs. Intense signal in the kidneys across all timepoints supports cy5.5-AAK₂₀ clearance by the renal system. FIG. 10B compares accumulation in the RV with the LV. In all animals, the signal in the RV stayed baseline compared with the healthy group. Still, the intensity of the infarcted LV increased post-MI in all animals, supporting the hypothesis of cy5.5-AAK₂₀ LV accumulation following systemic injection.

Methods 2.4: Evaluating Cytocompatibility of LOX-Responsive PLPs in vitro. The LOX-responsive PLPs were co-designed by the applicant and the Gianneschi lab, and were fabricated by the Gianneschi Lab for all studies using the same or substantially similar method described in Example 1. The PLP contain 20 norbornenes on the polymer backbone, and the LOX-responsive PLPs contain the substrate (AAKAAKAA). Cytocompatibility tests of LOX-responsive PLPs with L929 fibroblasts, primary rat cardiac fibroblasts and neonatal cardiomyocytes are performed. For initial testing, L929s are used, which are standard for assessing cytotoxicity. To evaluate PLP cytocompatibility on cardiac-specific cell lines, we then isolate primary rat cardiac fibroblasts and cardiomyocytes. Cells are pre-plated in a 96 well plate for at least 12 hours and are evaluated for initial cell health via an alamarBlue™ cell viability reagent. Afterward, we dose in LOX-responsive PLPs with concentrations from 1 μM to 100 μM to a 96 well plate and perform alamarBlue™ (n=3 replicate plates), as 30 μM is the expected material concentration once injected and diluted in the bloodstream.

Results 2.4: L929 fibroblasts, primary rat cardiac fibroblasts, and neonatal cardiomyocytes are all expected to demonstrate substantially similar absorbance values between those taken pre-treatment and absorbance values taken post-treatment. These results are expected to be consistent with all tested concentrations of PLPs, including 1 μM LOX-responsive PLPs, 30 μM LOX-responsive PLPs, and 100 μM LOX-responsive PLPs. The substantially similar absorbance values indicate a non-detectable change in cell viability when treated with a concentration of LOX-responsive PLPs expected in the bloodstream of a subject once injected. As such, these results will support that LOX-responsive PLPs are nontoxic to L929 fibroblasts, primary rat cardiac fibroblasts, and neonatal cardiomyocytes.

Methods 2.5: Characterizing the Morphology and Responsiveness of LOX-Responsive PLPs. LOX-responsive PLPs are generated by the same or substantially similar methods described in Example 1. Non-responsive PLPs have a scrambled substrate sequence assuring that the PLP is specifically reacting to LOX. To confirm that the LOX-responsive PLP can crosslink to itself and form a densely crosslinked scaffold, recombinant LOX enzyme is incubated with BMP-1, its activator, and the LOX-responsive PLP. To evaluate the morphology of the LOX-responsive PLP crosslinking onto itself, dynamic light scattering (DLS) is performed before and after incubating LOX-responsive PLPs and nonresponsive PLPs with activated LOX, or activated LOX and a LOX inhibitor, BAPN, each for 24 hours. Finally, to evaluate the morphology of the PLPs, LOX-responsive PLPs are pretreated with active LOX enzyme and a LOX inhibitor and are then visualized via transmission electron microscopy (TEM) to evaluate morphology change.

Results 2.5: Prior to enzymatic incubation, DLS findings show a LOX-responsive PLP diameter of <10 nm (FIG. 9B). Similarly, DLS is expected to show a diameter of <10 nm of non-responsive PLPs as well. After enzymatic incubation, DLS is expected show the presence of particle aggregates having diameters >10 nm in samples with LOX-responsive PLPs. There should be untraceable amounts, if any, aggregates in the samples with non-responsive PLPs. These results suggest that LOX-responsive PLPs are subject to enzyme regulated crosslinking, including crosslinks to itself, to form crosslinked scaffolds. Support for these findings can be shown in TEM images showing morphological changes in the LOX-responsive PLPs compared to the expected lack thereof in the non-responsive PLPs.

Methods 2.6: Evaluation of Targeting and Biodistribution of LOX-responsive PLPs in vivo. Female, Sprague Dawley rats undergo IR via temporary occlusion of the left coronary artery for 35 minutes. Cy5.5 conjugated LOX-responsive PLPs are administered intravenously via tail-vein (300 μM) post-MI at the following timepoints (n=3 per timepoint): 6 hours, 12 hours, 1 day, 3 days, or 7 days. All animals are euthanized 2 days post injection. For biodistribution studies, satellite organs are harvested where all organs are imaged on a LI-COR Odyssey to measure overall biodistribution of LOX-responsive PLPs with all quantification done via a custom MATLAB image analysis script. Hearts are excised, fresh frozen, and sectioned. To confirm localization in the infarct and colocalization to LOX, an antibody co-stain of LOX, collagen, elastin, and alpha-actinin to stain for cardiomyocytes alongside the LOX-responsive PLPs is performed. At 3 days post-MI, LOX and L-PLPs colocalize in the infarct, with negligible signal from LOX and L-PLPs in the remote myocardium (FIG. 12A).

Results 2.6: Compared to the RV, a significant increase in signal corresponding to LOX-responsive PLPs is expected to be present in the LV, which would be consistent with results shown in FIG. 10B for Results 2.3. The same level of signal increase is not expected in the healthy animal LVs, which evidences that the LOX-responsive PLPs may depend, at least in part, on the presence of upregulated LOX enzyme at the target site and/or leaky vasculature associated with the reperfused heart. Additionally, all animals are expected to exhibit high signal intensity corresponding to LOX-responsive PLPs in the kidneys, as exemplified in FIG. 10A showing the results from Results 2.3. This will further demonstrate PLP's disposition for renal clearance.

Methods 2.7: Evaluation of Retention Time of Responsive vs Non-Responsive PLPs in the Infarct in vivo. Female, Sprague Dawley rats undergo IR procedures as described in Methods 2.6 above. One day post-MI, animals are injected with either a Cy5.5 conjugated responsive PLP (n=3 per timepoint) or a Cy5.5 conjugated nonresponsive PLP (n=3 per timepoint). Retentions are studied at the following timepoints post injection: 2 days, 7 days, 14 days, and 28 days. At each timepoint, the heart is harvested. LI-COR Odyssey imaging of the heart and all satellite organs (kidneys, liver, spleen, and lungs) is performed. Histology is then done via hematoxylin and eosin (H&E) staining to confirm infarct size in rodents. Finally, antibody staining for LOX and alpha-actinin is performed.

Results 2.7: In animals treated with responsive PLPs, LI-COR results are expected to show significant signal strength in the LV in the hearts harvested 2 days post-injection, indicating a high retention rate of responsive PLPs in the LV 2 days after PLP injection (e.g., FIG. 10A). The hearts harvested 7 days, 14 days, and 28 days post-MI are expected to display a lower signal than the 2-day samples, with a decreasing signal strength over time (i.e., 14-day post-injection samples having a lower signal strength than 7-day post-injection samples). The decreasing signal strength combined with responsive PLP-CD68+ macrophage colocalization (e.g., FIG. 12B) provides evidence for macrophage mediated clearance via remodeling processes. The animals treated with nonresponsive PLPs are expected to have LI-COR results that show little, if any, signal in the LV across all timepoints. For both responsive and non-responsive PLPs, LI-COR results are expected to demonstrate high signal strength in the kidneys at 2 days post-injection, which is indicative of high retention of PLPs. The signal strength should weaken over time, with samples harvested 28-days post-injection having the lowest signal among the other timepoints. These results support renal excretion mediated clearance.

### Example 3

Methods 3.1: Development of LOX Crosslinkable PLP for Targeting MI in a Rat Ischemia-Reperfusion (IR) model. Responsive and non-responsive PLPs labeled with fluorophores are synthesized via solid phase support synthesis of peptides and living polymerization. Further physical characterization of Cy5.5-AAK₂₀ by SEC-MALS, dry state TEM, and SAXS provides complementary data to SDS PAGE, DLS, and DOSY NMR data. In vitro biological characterization of Cy5.5-AAK₂₀ is analyzed to establish proteolytic resistance, enzymology, toxicity, binding, hemocompatibility, cell viability, and immunogenicity. Cy5.5-AAK₂₀ is administered to infarcted rats via TV. The heart and satellite organs are analyzed by fluorescence imaging. Time course and retention studies are completed on the responsive PLPs to evaluate accumulation in the infarct at acute timepoints and retention over time.

Methods 3.2: Development of Therapeutic LOX Crosslinkable PLP for Targeting and Treating MI in a Rat IR model. Regeneration promoting therapeutic motifs, such as Nrf-2 or IL-10, are identified which have been shown to repolarize macrophages from pro-inflammatory phenotypes to anti-inflammatory phenotypes when uptaken intracellularly. Therapeutic-cy5.5-AAK₂₀ are synthesized following synthetic and characterization protocols described in Examples 1 & 2. Assess and optimize the therapeutic effect of therapeutic-cy5.5-AAK₂₀ through histological and fluorescent staining of ex vivo heart tissue and efficacy tests in vitro.

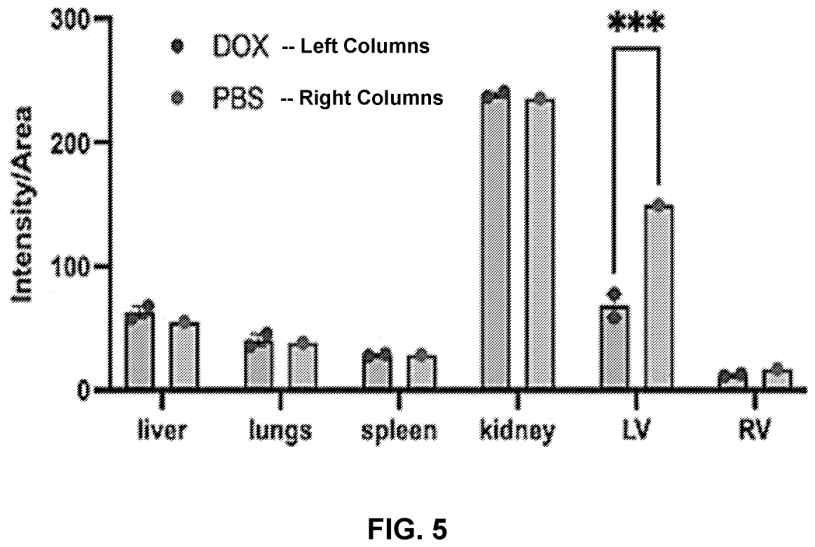

Methods 3.3: Optimization of LOX Crosslinkable PLP Physical Properties and Cardiac Biocompatibility. Computer simulations, together with small-angle X-ray scattering (SAXS) were performed to evaluate PLP physical properties. The functional domain of LOX enzyme (FIG. 6D) is considered when designing the LOX responsive substrate for the LOX-responsive PLP. D simulations predict the calcium-binding site of the LOX enzyme (FIG. 5D) tethers LOX-responsive substrates to the surface of the LOX enzyme. The sequence of the LOX responsive substrate is changed such that the crosslinked structure is more compliant. Reducing the number and density of crosslinkable lysine residues on each substrate as exemplified in FIG. 6C and/or on each PLP improves crosslinked PLP compliance. Utilizing amino acid sequences that assemble into coils as opposed to beta sheets reduces shear stress and improves elasticity. The norbornene imide PLP rigid backbone also contributes to ventricular stiffness and may be changed to a norbornene amide and/or a methylacrylamide or acrylamide backbone to improve crosslinked PLP elasticity and compliance with a LV wall (FIG. 6C). The elasticity of AAK₂₀ and experimental PLPs is measured by rheology.

### Example 4

The following exemplary methods evaluate the efficacy of a block copolymer PLP system in inhibiting matrix metalloproteinase (MMP). The block copolymer PLP system has 20 norbornenes on its ampiphilic copolymer backbone, a targeting agent configured to target the LOX enzyme having the sequence (AAKAAKAA), and a therapeutic agent corresponding to PD166793, which is configured to inhibit MMP activity. In this Example 4, this block copolymer PLP system is also referred to as “drug-loaded PLP,” “PLP-Loaded Drug,” or “L-PLPMMPi.” Block copolymer PLPs having all components described in this paragraph other than the therapeutic agent are referred to as “non-drug-loaded PLPs” or “L-PLPs.” The unattached therapeutic agent is referred to as “Free-Drug” or “MMPi.”

Methods 4.1: Confirming General Bioactivity in vitro with Free-Drug vs. PLP-Loaded Drug. First, a fluorometric hydrogen peroxidase-based LOX activity assay is used to confirm PLP activation by the LOX enzyme by incubating active LOX enzyme with L-PLP (1-100 μM). To evaluate the therapeutic potential of mitigating MMP activity, rat cardiac fibroblasts are isolated. Cells are then pre-plated and treated with phorbol myristate acetate (PMA) to induce MMP production as well as TGF-B to induce LOX production in vitro. Cells are then either treated with drug-loaded PLPs (L-PLPMMPi), non-drug-loaded PLPs (L-PLPs), or PBS (control). MMP activity is measured via a fluorescence resonance energy transfer (FRET) peptide-based assay (n=3 plates) 1 day post-treatment to confirm drug release.

Results 4.1: The cells treated with L-PLP_{MMPi} are expected to show a reduction in MMP activity compared to the cells treated with PBS control as well as those treated with L-PLPs. These results would suggest at least a portion of the PLP systems having a therapeutic (i.e., L-PLPMMPi) will be activated by the LOX enzyme to release the MMP inhibitor.

Methods 4.2: Evaluating Cytocompatibility and Hemocompatibility of L-PLPs in vitro. PLP cytocompatibility tests are performed with primary cardiac fibroblasts, neonatal cardiomyocytes, and macrophages. To evaluate PLP cytocompatibility on cardiac-specific cell lines, primary cardiac fibroblasts, neonatal cardiomyocytes, and differentiated macrophages are isolated. Cells are then pre-plated in a 96 well plate for 12 hours and are evaluated for initial cell viability via alamarBlue™ assays. Crosslinked L-PLPs and drug-loaded PLPs are dosed in at 1 μM to 100 μM with respect to polymer, given that 30 μM is the expected concentration in the bloodstream. AlamarBlue™ (n=3 replicate plates) assays are run. Finally, hemocompatibility studies are performed by measuring clotting times, platelet activation, and complement activation via PLP dosing at 1 μM to 100 μM with respect to polymer.

Results 4.2: Cardiac fibroblasts, neonatal cardiomyocytes, and macrophages are all expected to demonstrate substantially similar absorbance values taken pre-treatment and absorbance values taken post-treatment. These results are consistent with all tested concentrations of both crosslinked L-PLPs and drug-loaded PLPs, including concentrations 1 μM, 30 μM, and 100 μM. The substantially similar absorbance values indicate a non-detectable change in cell viability when treated with a concentration of PLPs expected in the bloodstream of a subject once injected. As such, these results will support that both L-PLPs and drug-loaded PLPs are nontoxic to cardiac fibroblasts, neonatal cardiomyocytes, and macrophages.

Hemocompatibility studies are expected to demonstrate no observable differences in clotting, platelet activation, nor compliment activation between the samples dosed with crosslinked L-PLPs and the samples dosed with drug-loaded PLPs.

Methods 4.3: Performing in vitro Efficacy Studies with L-PLPs via qPCR and Immunofluorescence. Cardiac fibroblasts are seeded and treated with PMA treatment and L-PLPMMPi dosing at 1 μM to 100 μM with respect to polymer. To assess drug efficacy, RNA is isolated and qPCR is performed for MMP2, MMP9, interleukin 6 (IL6), interleukin 10 (IL10), and interleukin 4 (IL4) to thus demonstrate the immunomodulatory properties of L-PLP_{MMPi}. Finally, caspase 3 is stained for, to show how L-PLP_{MMPi} affects cellular survival.

Results 4.3: For cardiac fibroblasts treated with L-PLP_{MMPi}, reduced expression of MMP2, MMP9, and IL6 is observed compared to the fibroblasts treated with PMA control. Since MMP2, MMP9, and IL6 (pro-inflammatories) are known to be upregulated in infarcted myocardium, their reduced expression suggests L-PLP_{MMPi} facilitates the induction of anti-inflammatory processes. In contrast, IL10 and IL4 are shown to have increased expression when treated with L-PLP_{MMPi} compared to PMA control. Since IL10 and IL4 code for anti-inflammatory cytokines, their increased expression supports the conclusion that L-PLP_{MMPi} facilitates the induction of anti-inflammatory processes.

Additionally, caspase 3 stains are expected to demonstrate reduced presence of caspase 3 in cardiac fibroblasts treated with L-PLP_{MMPi} compared to fibroblasts treated with PMA control. Since caspase 3 protein plays a substantial role in the induction of cell apoptosis, its reduction corresponds to a reduction in apoptosis providing evidence that L-PLP_{MMPi} facilitates promotion of cellular survival.

### Example 5

The following exemplary methods further evaluate the efficacy of a block copolymer PLP system in inhibiting matrix metalloproteinase (MMP) in vivo. The block copolymer PLP system has 20 norbornenes on its amphiphilic copolymer backbone, a targeting agent configured to target the LOX enzyme having the sequence (AAKAAKAA), and a therapeutic agent corresponding to PD166793, which is configured to inhibit MMP activity. In this Example 5, this block copolymer PLP system is also referred to as “drug-loaded PLP,” “PLP-Loaded Drug,” or “L-PLP_{MMPi}.” Block copolymer PLPs having all components described in this paragraph other than the therapeutic agent are referred to as “non-drug-loaded PLPs” or “L-PLPs.” The unattached therapeutic agent is referred to as “Free-Drug” or “MMPi.”

Methods 5.1: Assessing Efficacy of L-PLP_{MMPi} on LV Function. Male and female Sprague Dawley rats undergo IR procedures as previously described in Example 2. Prior to surgery, all animals undergo magnetic resonance imaging (MRI) to establish a baseline for LV ejection fraction (EF), wall thickness, end diastolic volume (EDV), and end systolic volume (ESV). Prior to tail-vein injections 1-day post-MI, animals that do not have an EF at least 1 standard deviation below healthy values (<68%) are excluded from the study as evidenced by echocardiography. MRI is performed at 3 days and 5 weeks (n=7 per sex for saline, MMPi, L-PLP, and L-PLP_{MMPi} treated animals with randomized groups for each harvest timepoint) post-injection to assess cardiac function via LV EF, wall thickness, EDV, and ESV. For power analysis, an a value of 0.05 and a power of 0.8 was used to determine experimental sample sizes with additional rats based on an expected 30% mortality from surgical procedures. A 2-way ANOVA with post-hoc Tukey Test is then performed with significance at α=0.05.

Results 5.1: Animals treated with L-PLP_{MMPi} are expected to depict negligible differences between pre- and post-MI measurements of wall thickness, EF, EDV, and ESV. It is expected that animals treated with L-PLP will depict negligible differences between pre- and post-MI measurements of wall thickness. However, L-PLP samples are expected to depict decreased EF and EDV values post-MI and increased ESV values post-MI compared to the pre-MI counterparts. Animals treated with MMPi are expected to depict negligible differences between pre- and post-MI measurements of wall thickness. However, it is anticipated that MMPi samples will depict decreased EF values and increased EDV and SESV values post-MI compared to the pre-MI counterparts. Finally, as expected with the saline negative control, saline-treated samples should depict decreased wall thickness, EF, EDV, and ESV values post-MI compared to the pre-MI saline counterparts.

Methods 5.2: Confirming Therapeutic Efficacy via Histological Analysis and MMP Activity. Using the same hearts or same preparation of hearts from Methods 5.1, hearts from animals treated with L-PLP_{MMPi} and animals treated with saline are excised 3 days post injection and used for MMP activity assay studies to confirm inhibition. Hearts from animals treated with L-PLP_{MMPi} and animals treated with saline from 5 weeks post injection are harvested and stained with hematoxylin and eosin to assess infarct size and Masson's trichrome to evaluate fibrosis. Infarct size, percent fibrosis in the infarct, and interstitial fibrosis in the remote myocardium are then compared.

Results 5.2: MMP activity assays are expected to show that animals treated with L-PLP_{MMPi} have lower MMP activity compared to samples from animals treated with the saline control. Additionally, animals treated with L-PLP_{MMPi} are expected to depict decreased infarct size, decreased percent fibrosis in the infarct, and decreased interstitial fibrosis in the remote myocardium compared to animals treated with saline controls. These results suggest that L-PLP_{MMPi} mitigate negative LV remodeling (i.e., decreased infarct size, decreased percent fibrosis, and decreased interstitial fibrosis) by inhibiting MMP2 and MMP9 (i.e., lower MMP activity) and preserving EF.

### Example 6

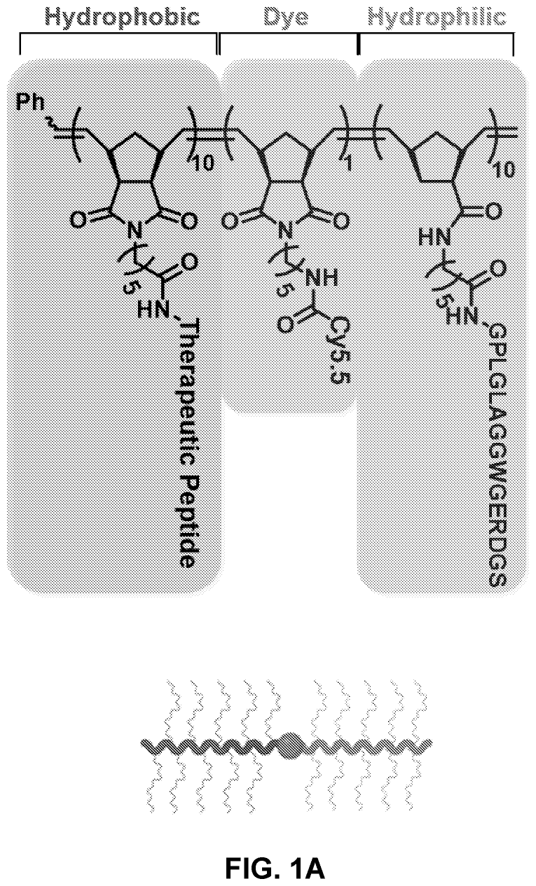

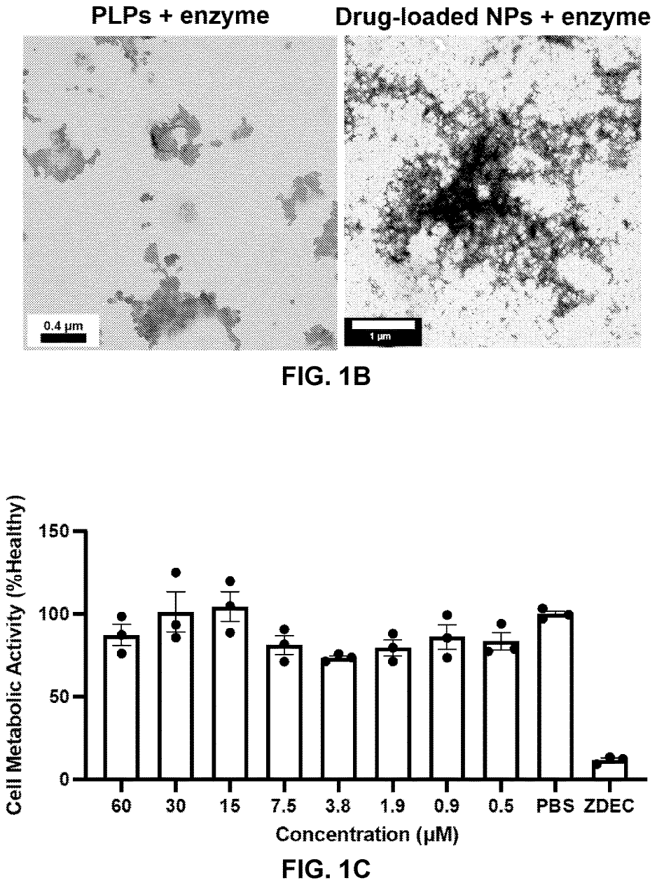

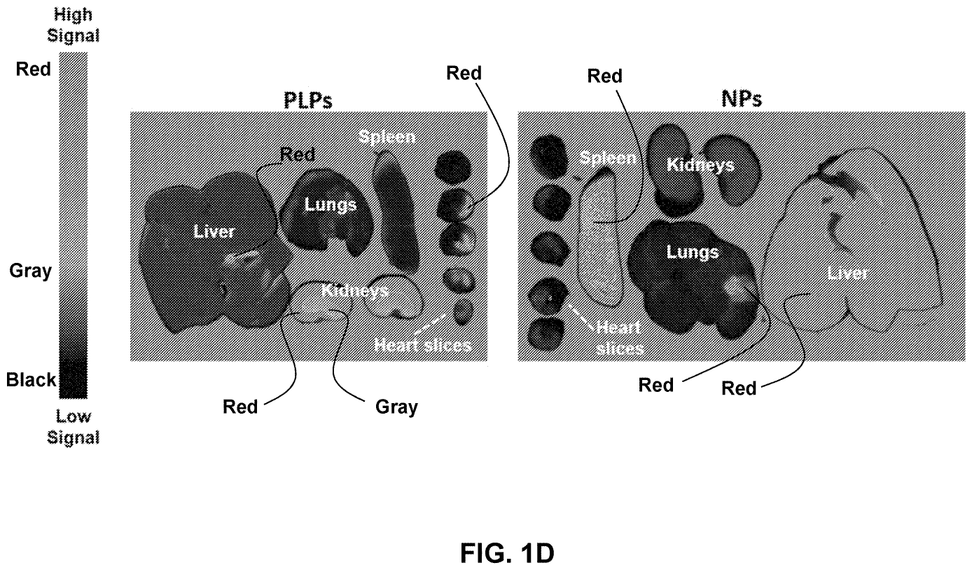

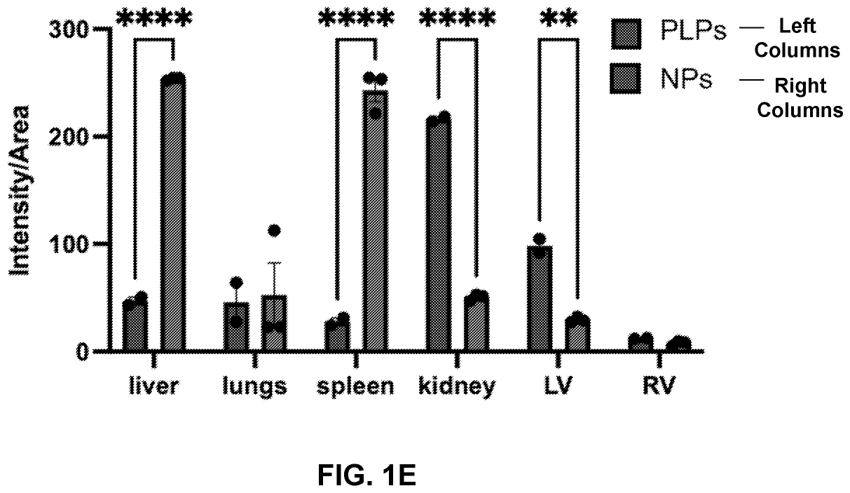

The following methods provide an exemplary block copolymer PLP system capable of targeted delivery to regions of inflammation in a subject, namely to infarcted regions of a heart. Retention, cytocompatibility, and therapeutic efficacy studies, among others, are provided below to characterize the exemplary block copolymer. The block copolymer PLP system has an amphiphilic polynorbornene backbone, a targeting agent configured to target MMP enzyme (i.e., MMP-responsive), and a therapeutic agent, which is configured to inhibit MMP activity. FIG. 1A provides an example of an MMP-responsive PLP formula employed in this Example 6.

### Procedures:

Surgical Procedures and IV Injection. All procedures in this study were approved by the Committee on Animal Research at the University of California, San Diego and the Association for the Assessment and Accreditation of Laboratory Animal Care. Female, Sprague Dawley rats (225-250 g) underwent ischemia-reperfusion (TR) procedures via left thoracotomy and temporary occlusion of the left anterior descending artery for 35 minutes. One day post-MI, animals were anesthetized using isoflurane and randomly intravenously injected with 1 mL of PLPs (300 μM with respect to polymer) and harvested at 1, 7, 21, and 28 days post-injection (n=3 for each timepoint). Animals were euthanized via overdose of pentobarbital (200 mg/kg) and the satellite organs (kidney, spleen, lungs, and liver) were collected for LI-COR analysis. The heart was excised and matrix sliced into 5 sections for LI-COR analysis and immunohistochemistry. This process was repeated for all control materials as well but animals were only harvested at the 1 day post-injection timepoint.

Methods 6.1: MMP-Responsive PLPs Infarcted Heart Retention and Avoidance of Satellite Organ Accumulation. PLPs (100 M, with respect to polymer) were treated with thermolysin, an MMP alternative with improved thermostability, (1 μM) or DPBS for 24 hours at 37° C. in 1X DPBS. The resulting nanoparticle solutions were analyzed by dynamic light scattering (DLS) and transmission electron microscopy (TEM) to examine the change in morphology. For the TEM samples, 5 μL of sample was applied to a 400-mesh carbon grid (Ted Pella, Inc.) that had been glow discharged for 15 seconds. 5 L of 2 wt. % uranyl acetate solution was then applied and wicked away post 30 sec for staining.

Results 6.1: Formation of micron-scale aggregates were observed following MMP enzymatic cleavage (FIG. 1B, “PLPs+enzyme”).

Methods 6.2: MMP-responsive PLP Cytocompatibility. For cytocompatibility assessment, murine fibroblast cells (L929) were used in accordance with the UNI ISO 10993/2009 for cytotoxicity assays. Cells were plated and left to adhere overnight. Following cell adhesion, MMP-Responsive PLPs were added at physiologically relevant concentrations spanning 60-0.5 μM with PBS and zinc diethyldithiocarbamate (ZDEC) serving as positive and negative controls, respectively. Here, the maximum concentration was chosen to be higher than that the initial dilution of drug-loaded nanoparticles (DNPs) into the blood volume of a rat, in this case, ˜17 μM. Treated cells were then incubated for 24 hours before performing an alamarBlue™ assay to evaluate their metabolic activity. All treatments were normalized to the healthy PBS control.

Results 6.2: No significant decrease in metabolic activity compared to a healthy control was observed (FIG. 1C).

Methods 6.3: MMP-Responsive PLP Localization. The methods performed to measure MMP-Responsive PLP localization generally correspond to “Surgical Procedures and IV Injection” described above under “Procedures.”

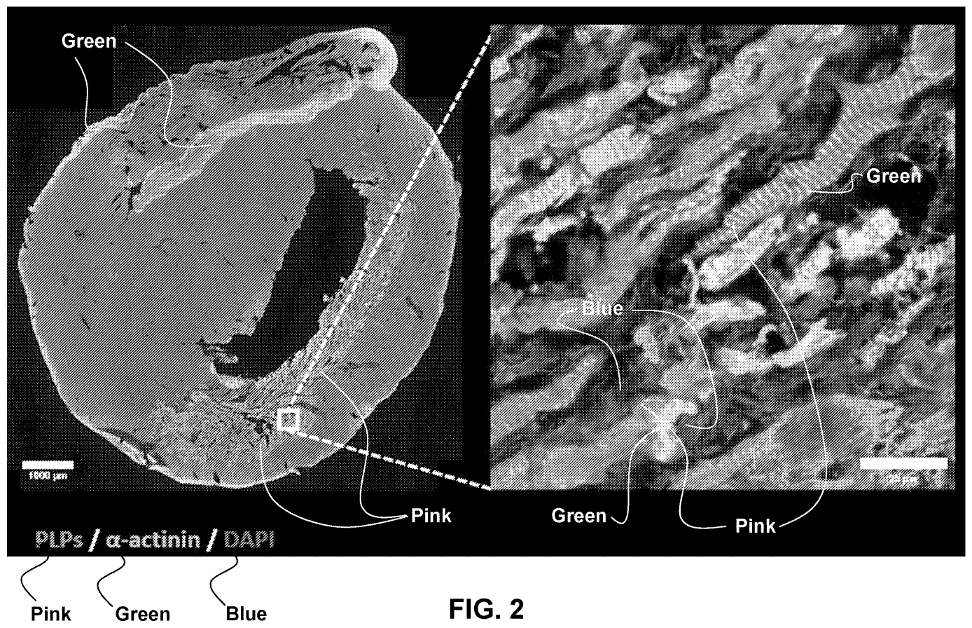

Results 6.3: At one day post-injection, strong PLP localization in the infarcted region of the heart was observed. Similar to nanoparticle platforms, the PLPs exhibited the most accumulation in the infarct with little material retention in the remote myocardium and borderzone. (FIG. 2) The morphology of PLPs resembled a bolus style injection with strong signal and spread throughout the entire infarct. Negligible amounts of material localized to the borderzone and remote myocardium, but that which did have more rounded, punctate morphology. Higher resolution imaging demonstrates that PLPs very specifically localized to the infarcted region of the left ventricle and appear to colocalize with cardiomyocytes (i.e., DAPI in FIG. 2) within the necrotic core of the infarct (FIG. 2, right).

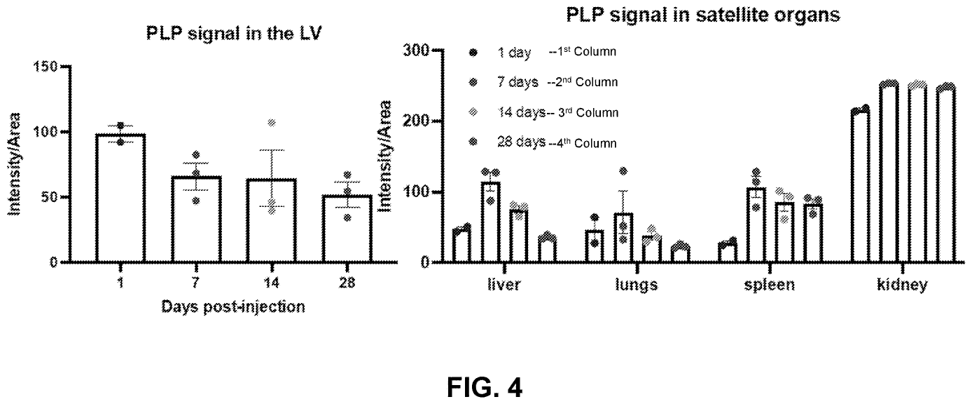

PLP biodistribution at one day post-injection was very concentrated in the kidneys and infarcted region of the heart (FIG. 4), this trend continued at one- and two-weeks post-injection with some increased signal in the liver as well (FIG. 4). At 4 weeks post-injection, a decrease in signal in the left ventricle was observed but no change in PLP signal in the kidneys (FIG. 4). Quantification of LI-COR scans showed that though PLP signal shows a decrease over time in the left ventricle (FIG. 4, left), there was no significant difference in this signal over time. Looking at the satellite organs, we confirmed the presential retention of PLPs in the kidneys over the liver and spleen at all timepoints (FIG. 4, right). Additionally, a decrease in signal in the liver, lungs, and spleen over time, but not the kidneys was observed.

Methods 6.4: MMP-Responsive PLP Biodistribution and Cellular Uptake. To assess the ability of MMP-Responsive PLPs to be uptaken by necrotic cells in vitro, neonatal cardiomyocytes were treated with 500 μM H₂O₂ for 3 hours to induce inflammation that mimics the infarct environment. Cells were then treated overnight with 50 μL of MMP-Responsive PLPs amounting to a final well concentration of 17 μM. After 24 hours of PLP incubation, cells were stained with propidium iodide (PI), which is known to stain necrotic nuclei, and calcein am to identify necrotic and live cells, respectively, and imaged on a Keyence BZ-X microscope. To determine PLP localization to necrotic cells in vivo, PI was resuspended in PBS at 100 mg/mL. That solution was then used to resuspend PLPs at 300 μM and sterile filtered for injection. As before, animals who had undergone IR injury one day prior then received a 1 mL of PI+PLPs and were harvested 24 hours post-injection. Hearts were excised and frozen in OCT for cryosectioning.

Results 6.4: After induction of inflammation via H₂O₂ treatment, PLP incubation, and staining, strong colocalization of PLPs by cells that stained positively for calcein and propidium iodide was observed. Looking just at the Cy5.5 channel, representing fluorescently tagged PLPs, the material appeared to be filling in the cell body, indicating material uptake. Colocalization of PLPs and necrotic cells was confirmed by looking at the merged image where all channels appeared to overlap. When a co-injection of PLPs and propidium iodide in vivo one day post-MI was administered, microscopy of these infarcts revealed strong colocalization of propidium iodide positive nuclei with Cy5.5-labeled material in the infarcted region of the heart.

Methods 6.5: Comparison of MMP-Responsive PLP Biodistribution and Cellular Uptake in vivo with MMP-Nanoparticles.

Results 6.5: When comparing this platform to MMP-responsive nanoparticles, we noticed stark differences in biodistribution. Generally, PLPs demonstrate a higher degree accumulation in the kidneys and infarct (FIG. 1D, left) whereas the nanoparticles appear to accumulate more in satellite organs, such as the spleen and liver (FIG. 1D, right). These visual differences were confirmed via quantification of the organ scans (FIG. 1E).

Methods 6.6: Evaluation of Role MMP Activity Plays in PLP Accumulation. To determine how important the MMP-responsive nature of this material was to accumulation in the heart, we aimed to quell MMP activity through a one-time intraperitoneal (IP) injection of doxycycline hyclate (DOX). One day post-MI, animals received an IP injection of doxycycline hyclate (DOX, Sigma) at a dose of 100 mg/kg. Two hours following administration of DOX, when the concentration of DOX was expected to be peaking in serum concentration, animals underwent a tail vein injection 1 mL of PLPs (300 μM) and were then harvested 3 hours later. The satellite organs (kidney, spleen, lungs, and liver) were collected for LI-COR analysis, and heart was excised and matrix sliced into 5 sections for LI-COR analysis and immunohistochemistry.

Results 6.6: Quantification of LI-COR scanned satellite organs and heart slices showed a significant decrease in PLP accumulation in the heart when DOX was administered compared to a PBS control (FIG. 5). We observed a significant difference in PLP accumulation in the LV but no differences in accumulation in any of the satellite organs for either group (FIG. 5). The results suggest that MMP-responsive peptide sequence not only provides the PLP an amphiphilic nature but also induces a morphological switch once it is cleaved. These results suggest that the MMP-responsive peptide sequence is relevant for targeting and material retention in areas of interest.

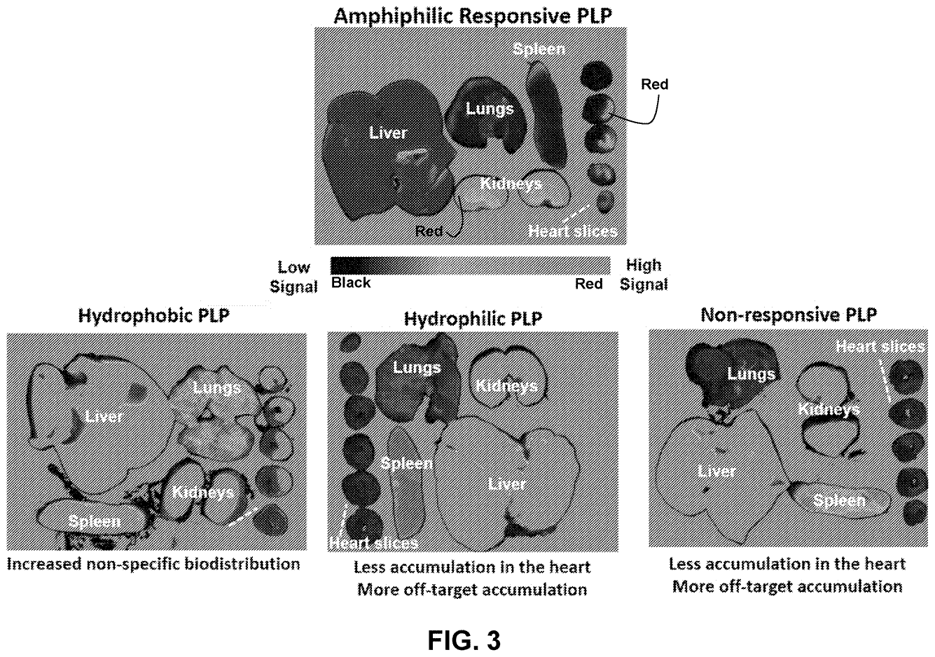

Methods 6.7: MMP-Responsive PLP Control Materials. Various controls were run to better understand the mechanisms by which MMP-Responsive PLPs accumulated in the heart and displayed favorable biodistribution in satellite organs. Comparing all control groups to the biodistribution of the original PLP material (FIG. 3), we first injected healthy animals with PLPs and observed no signal in the heart slices and much more material retention in the satellite organs (liver, spleen, and kidneys) (FIG. 3). We observed a similar trend in biodistribution with a non-responsive control, a control where we removed the MMP-responsive sequence of the cleavable peptide and instead inserted a glycine-serine spacer (FIG. 3, bottom right). Administration of the therapeutic block only, without the MMP-responsive block resulted in high PLP accumulation in all the satellite organs in addition to the infarcted heart (FIG. 3, bottom left). Quantification of all these groups further demonstrated the difference in how altering the PLP drastically affects the biodistribution, specifically in the kidneys, liver, spleen, and heart (FIG. 3). These results suggest that MMP-responsiveness and amphiphilic material plays a role in accumulation in the heart and favorable biodistribution in satellite organs.

### Example 7

Methods 7.1: MMP-Responsive PLP Regulation of Downstream Cellular Processes. A custom Nanostring cardiac rat panel (374 genes) was run to compare the overall downstream genetic differences between MMPi responsive PLPs and saline in a rat MI model. Animals were subjected to MI as mentioned previously, and treated with MMPi responsive PLP (N=6) or saline (N=6) 1 day post-MI. Hearts were harvested 4 days post-injection (total 5 days post-MI). RNA was isolated from even matrix slices of heart tissue. The Volcano Plot was analyzed with the Enhanced Volcano package in R software with (p<0.05, −0.25<Avg2FC<0.25) between conditions. Enrichr was then used to determine enrichment of differentially expressed genes from samples treated with MMPi-Responsive PLPs.

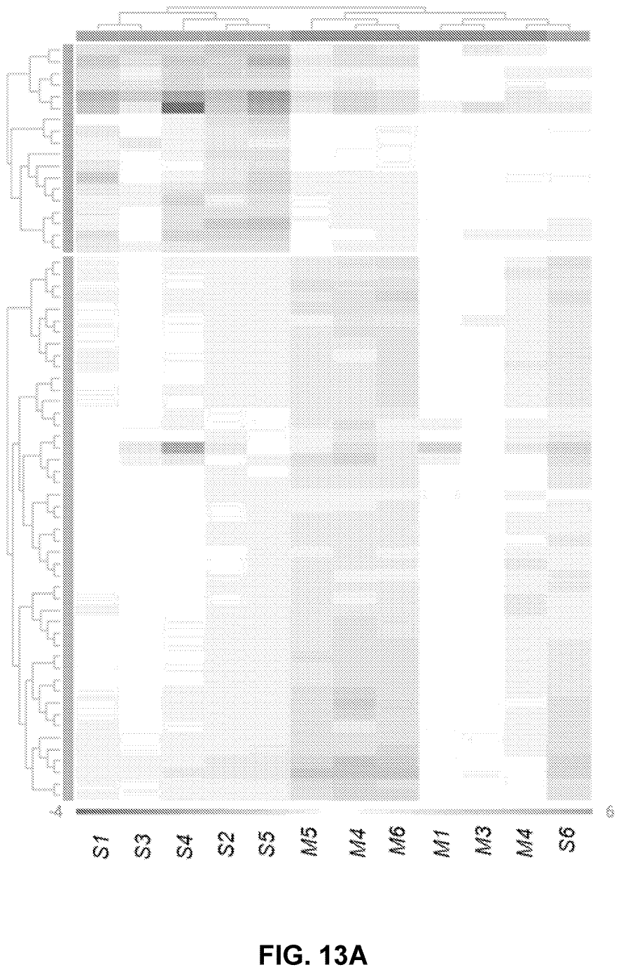

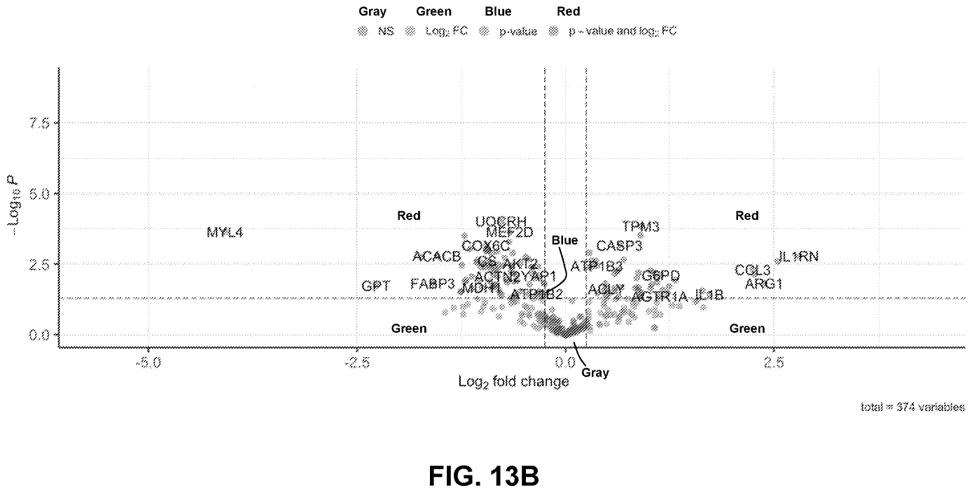

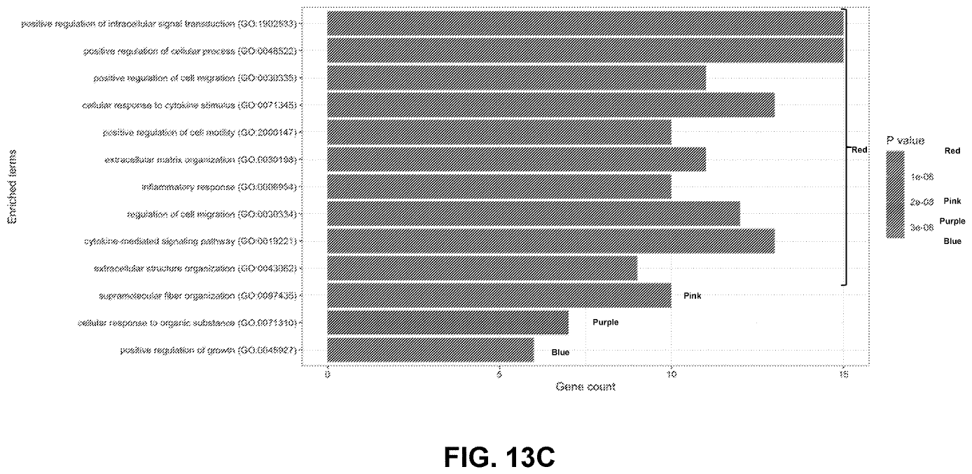

Results 7.1: The Volcano Plot of FIG. 13A highlights 133 genes that were differentially expressed. The differentially expressed genes highlighted in the Volcano Plot (FIG. 13A) are further displayed in FIG. 13C. While in general extracellular matrix organization and positive regulation of cell growth and motility were found via enrichment as a result of MMP inhibition, we noted similar high presence of Tgfb1, Tgfb2, Arg1, Il1rn, and Tgfbr1, as shown in FIG. 13B. Upregulation of those genes demonstrates the MMPi responsive PLP is involved in shifting the immune response towards an anti-inflammatory phenotype by reducing the inflammation present due to MI pathophysiology.

### Example 8

Examples of therapeutic agents directed to Keap1/Nrf2 interactions herein are described in detail, including descriptions of exemplary synthesis, three-dimensional structures, and binding interactions, in PCT Patent App. No. PCT/US2022/023274, which is hereby incorporated by reference in its entirety.

### STATEMENTS REGARDING INCORPORATION BY REFERENCE AND VARIATIONS

(1) All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

(2) The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be clear to one of skill in the art, methods and devices useful for the present methods may include a large number of optional composition and processing elements and steps.

(3) When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

(4) It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

(5) Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. Nothing herein is to be construed as an admission that the invention is not entitled to antedate such disclosure by virtue of prior invention.

(6) Whenever a range is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range, unless otherwise indicated. For example, a range of 1 to 100 specifically includes the end point values of 1 and 100. It will be understood that any subranges or individual values in a range or subrange that are included in the description herein can be excluded from the claims herein.

(7) As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and 25 “consisting of” may be replaced with either of the other two terms.

(8) One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

(9) All references, including publications, patent applications, and patents, cited herein are hereby incorporated by reference to the same extent as if each reference were individually and specifically indicated to be incorporated by reference and were set forth in its entirety herein.

(10) The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

(11) Specific embodiments of this invention are described herein. However, variations of those specific embodiments will become apparent to those of ordinary skill in the art upon reading the foregoing description. It is generally recognized, therefore, that skilled artisans may employ such variations as appropriate. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The invention claimed is:

1. A block copolymer characterized by a formula (FX1a): 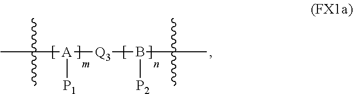
wherein
each A is independently a first backbone monomer;
each B is independently a second backbone monomer, wherein B is more hydrophilic than A;
m is an integer selected from the range of 1 to 1000;
n is an integer selected from the range of 1 to 1000;
Q₃ is optionally present and is a linking group, optionally one or more polymer grafting groups;
each P₁ is optionally present and is a therapeutic agent, optionally a therapeutic peptide, provided that at least one P₁ is present, and optionally a plurality of P₁ is present; and
each P₂ is optionally present and is a targeting agent, optionally a targeting peptide, provided that at least one P₂ is present, and optionally a plurality of P₂ is present.

2. The block copolymer of claim 1, wherein the block copolymer is characterized by a formula (FX1b): 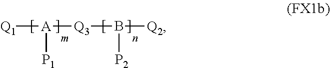
wherein Q₁ and Q₂ are each independently a polymer block terminating group.

3. The block copolymer of claim 1, wherein monomer [A(P₁)] is characterized by a formula (FX1c): 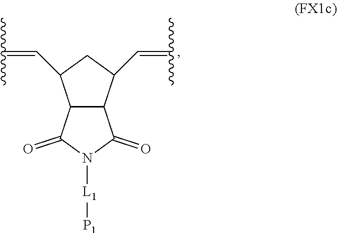
wherein L₁ is optionally present and is a first linking group; wherein monomer [B(P₂)] is characterized by a formula (FX1d) or (FX1e): 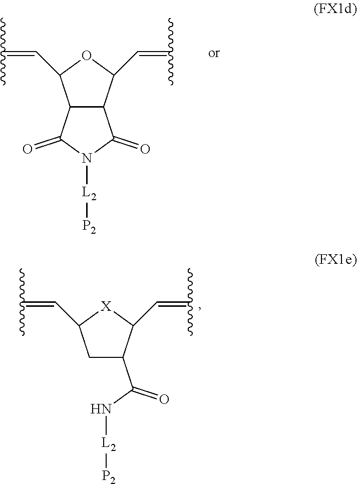
wherein L₂ is optionally present and is a second linking group, and X is CH₂ or O; or
any combination thereof.

4. (canceled)

5. (canceled)

6. The block copolymer of claim 1, wherein the block copolymer comprises a first backbone monomer characterized by a formula (FX1c1), (FX1c2), or (FX1c3): 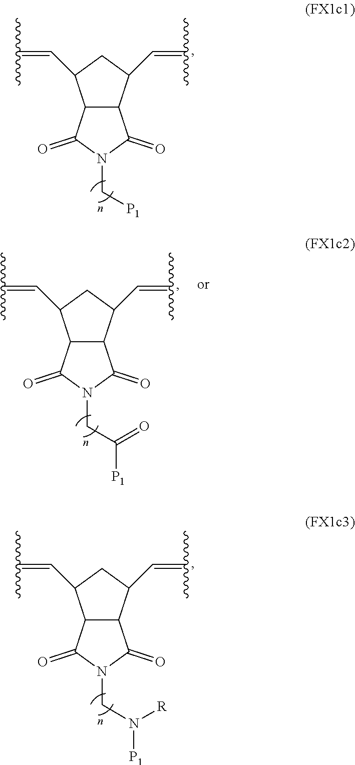
wherein n is an integer from 1 to 20 and R is a hydrogen or a C₁-C₅ alkyl; wherein the block copolymer comprises a second backbone monomer characterized by a formula (FX1d1), (FX1d2), (FX1d3), (FX1e1), (FX1e2), or (FX1e3): 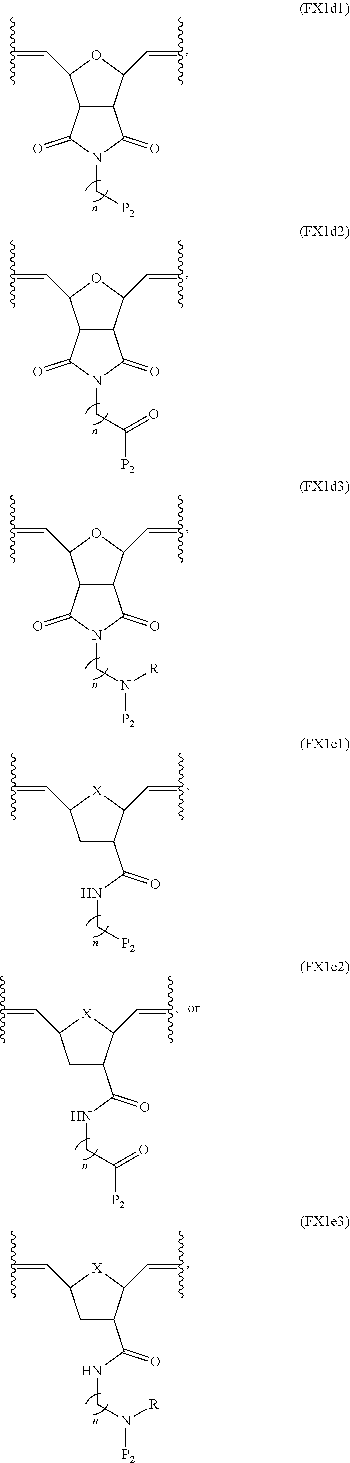
wherein n is an integer from 1 to 20, X is CH₂ or O, and R is hydrogen or a C₁-C₅ alkyl: or any combination thereof.

7. (canceled)

8. (canceled)

9. The block copolymer of claim 3, wherein each of L₁ and L₂ is independently selected from a single bond, an oxygen, and groups having an alkylene group, a heteroalkylene group, an alkenylene group, an arylene group, an alkoxy group, an acyl group, a triazole group, a diazole group, a pyrazole group, —O—, C₁-C₁₀ alkyl, C₂-C₁₀ alkylene, C₁-C₁₀ heteroalkylene, C₃-C₁₀ arylene, C₁-C₁₀ alkoxy, C₁-C₁₀ acyl, a C₁-C₂₀ alkylene and C₁-C₂₀ heteroalkylene, optionally terminated in a carbonyl, an amine, or an amide, —(CH₂)ₙNR—, —(CH₂)ₙC(O)NR—, —(CH₂)ₙNRC(O)—, —(CH₂)ₙC(O)— and —(CH₂)ₙ—, wherein n is an integer from 1 to 20 and R is hydrogen or a C₁-C₅ alkyl, and combinations thereof.

10. (canceled)

11. (canceled)

12. (canceled)

13. (canceled)

14. The block copolymer of claim 3, wherein each of L₁ and/or L₂ further comprises an enzymatically degradable linker, and wherein at least a portion of each of P₁ and P₂ is independently linked to the enzymatically degradable linker: wherein the enzymatically degradable linker is a MMP cleavage sequence, a cathepsin B cleavage sequence, an ester bond, a reductive sensitive bond-disulfide bond, a pH sensitive bond-imine bond, a carbamate, or any combination thereof.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. The block copolymer of claim 1, wherein P₁ comprises a therapeutic peptide having a chain length of 3 to 100 amino acid residues, P₂ comprises a targeting peptide having a chain length of 3 to 100 amino acid residues, or any combination thereof.

31. (canceled)

32. (canceled)

33. (canceled)

34. (canceled)

35. (canceled)

36. (canceled)

37. (canceled)

38. (canceled)

39. (canceled)

40. The block copolymer of claim 1, wherein the block copolymer has a length of less than or equal to 20 nm.

41. (canceled)

42. The block copolymer of claim 1, wherein the block copolymer comprises a formula configured to form crosslinkages with itself, wherein the crosslinkages comprise disulfide bonds: wherein the formation of crosslinkages with itself results in a block copolymer scaffold, or any combination thereof.

43. (canceled)

44. (canceled)

45. (canceled)

46. (canceled)

47. (canceled)

48. (canceled)

49. (canceled)

50. The block copolymer of claim 1, wherein P₂ comprises a net cationic sequence, P₂ targets a surface membrane protein, P₂ targets an extracellular receptor or a transmembrane receptor, P₂ targets an enzyme, P₂ comprises a tumor-targeting sequence, or any combination thereof.

51. (canceled)

52. (canceled)

53. (canceled)

54. The block copolymer of claim 1, wherein P₂ comprises an interleukin-6 inhibitor, an interleukin-10 inhibitor, a JAK/STAT inhibitor, or a LOX inhibitor.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. The block copolymer of claim 1, wherein P₂ comprises a sequence having 75% or greater sequence identity of SEQ ID NO: 142 (AAKAAKAA), wherein the sequence having 75% or greater sequence identity of SEQ ID NO: 142 (AAKAAKAA) has a point mutation to comprise an arginine residue, wherein the sequence having 75% or greater sequence identity of SEQ ID NO: 142 (AAKAAKAA) has a point mutation to comprise a glutamate residue, or any combination thereof.

68. (canceled)

69. (canceled)

70. (canceled)

71. (canceled)

72. (canceled)

73. (canceled)

74. The block copolymer of claim 1, wherein P₂ comprises a sequence having 75% or greater sequence identity of SEQ ID NO: 150 (GGGGEKGGGGG), wherein the sequence having 75% or greater sequence identity of SEQ ID NO: 150 (GGGGEKGGGGG) has a point mutation to comprise a glutamine residue, or any combination thereof.

75. (canceled)

76. (canceled)

77. (canceled)

78. (canceled)

79. (canceled)

81. (canceled)

82. (canceled)

83. The block copolymer of claim 1, wherein P₂ comprises a sequence having a length selected from the range of 4.8 Å to 14.2 Å, P₂ comprises a sequence having at least one lysine residue, P₂ comprises a sequence having at least one methylated lysine residue, or any combination thereof.

84. (canceled)

85. (canceled)

86. The block copolymer of claim 1, wherein P₂ comprises a sequence having 75% or greater sequence identity of:
SEQ ID NO: 154
(GPLGLAGGWGERDGS);
SEQ ID NO: 155
(GGSGSGSGWGERDGS);
SEQ ID NO: 214
(CSTSMLKAC);
or
SEQ ID NO: 216
(CPDRSVNNC).

87. (canceled)

88. (canceled)

89. (canceled)

90. (canceled)

91. (canceled)

92. (canceled)

93. (canceled)

94. (canceled)

95. The block copolymer of claim 1, wherein each P₁ independently treats or manages a condition of a subject, wherein each P₁ independently treats or manages a neurodegenerative disease, inflammation, an inflammatory disease, myocardial infarction, cancer, a musculoskeletal disease, or any combination thereof.

96. (canceled)

97. (canceled)

98. (canceled)

99. The block copolymer of claim 1, wherein P₁ comprises a sequence having 75% or greater sequence identity of SEQ ID NO: 1 (LDEETGEFL), wherein the sequence having 75% or greater sequence identity of SEQ ID NO: 1 (LDEETGEFL) has a point mutation to comprise a proline residue, wherein the sequence having 75% or greater sequence identity of SEQ ID NO: 1 (LDEETGEFL) has a point mutation to delete a glutamate residue, or any combination thereof.

100. (canceled)

101. (canceled)

102. (canceled)

103. (canceled)

104. (canceled)

105. (canceled)

106. (canceled)

107. The block copolymer of claim 1, wherein at least one, and optionally all, of P₁ comprises a compound having the formula of C₁₇H₁₈BrNO₄S, C₂₂H₂₄N₂O₈, or any combination thereof.

108. (canceled)

109. The block copolymer of claim 1, wherein P₁ comprises a sequence having 75% or greater sequence identity of:
SEQ ID NO: 156
(ISYGNDALMP);
SEQ ID NO: 157
(SLINGPAYMD);
TIMP-1;
SEQ ID NO: 158
(GACLRSGRGCG);
SEQ ID NO: 159
(GAALRSGRGAG);
SEQ ID NO: 160 (PRCCGE), wherein a biphenylalanine (B) is optionally present between the third and fourth amino acid residues of the sequence having 75% or greater sequence identity of SEQ ID NO: 160:
SEQ ID NO: 161
(MCMPCFTTDHQMARKCDDCCGGKGRGKCYGPQCLCR);
SEQ ID NO: 162
(HWWQWPSSLQLRGGGS);
SEQ ID NO: 163
(HNWTRWLLHPDRGGGS);
SEQ ID NO: 164
(GACFSIAHECGA);
SEQ ID NO: 165
(VMDGYPMP),
wherein the sequence having 75% or greater sequence identity of SEQ ID NO: 165 (VMDGYPMP) comprises an acetyl group attached to the N-terminus of the sequence identity and an amino group attached to the C-terminus of the sequence identity.

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. The block copolymer of claim 1, wherein P₁ comprises a sequence having 75% or greater sequence identity of:
SEQ ID NO: 166 (GYPKSALR), wherein the sequence having 75% or greater sequence identity of SEQ ID NO: 166 (GYPKSALR) comprises an acetyl group attached to the N-terminus of the sequence identity and an amino group attached to the C-terminus of the sequence identity;
SEQ ID NO: 167
(NENLLRFFVAPFPEV);
SEQ ID NO: 168
(CRVYGPYLLC);
SEQ ID NO: 169
(ADGACGYGRFSPPCGAAG);
SEQ ID NO: 170
(ADGACILWMDDGWCGAAG);
SEQ ID NO: 171
(STTHWGFTLC);
SEQ ID NO: 172
(SLRRSSCFGGRMDRIGAQSGLGCNSFRY);
SEQ ID NO: 173
(SLRRSSCFGGRIDRIGAQSGLGCNSFRY);
SEQ ID NO: 174
(MGSFSITLGFFLVLAFWLPGHIGPNPVYSAVSNTD);
SEQ ID NO: 175
(SQGSTLRVQQRPQNSKVTHISSCFGHKIDRIGSVSRLGCNALKLL);
SEQ ID NO: 176
(NSKMAHSSSCFGQKIDRIGAVSRLGCDGLRLF)
SEQ ID NO: 177
(SPKMVQGSGCFGRKMDRISSSSGLGCKVLRRH);
SEQ ID NO: 178
(GLSKGCFGLKLDRIGSMSGLGC);
SEQ ID NO: 179
(EVKYDPCFGHKIDRINHVSNLGCPSLRDPRPNAPSTSA);
SEQ ID NO: 180
(MAKSGIYLGCFILILIQNMVA);
SEQ ID NO: 181
(AMVSEFLKQAWFIENEEQEYVQTVK);
SEQ ID NO: 182
(TAPRSLRRSSCFGGRMDRIGAQSGLGCNSFRY);
SEQ ID NO: 183
(DNKPPKKGPPNGCFGHKIDRIGSHSGLGCNKVDDNKG);
SEQ ID NO: 224
(CTTHWGFTLC);
SEQ ID NO: 225
(RPKPQQFFGLM);
or
SEQ ID NO: 223
(GYGSSSRRAPQT).

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. The block copolymer of claim 1, wherein P₁ comprises a sequence having 75% or greater sequence identity of Annexin-A1 N-Terminal Peptide, a VEGF peptide, a ghrelin peptide, or any combination thereof.

143. (canceled)

144. (canceled)

145. (canceled)

146. The block copolymer of claim 1, wherein P₁ further comprises a charge modulating domain, the charge modulating domain has from 2 to 7 amino acid residues, the charge modulating domain is a glycine-serine domain, the charge modulating domain is a cationic residue domain, or a combination thereof.

147. (canceled)

148. (canceled)

149. The block copolymer of claim 1, wherein P₁ has from 11 to 16 amino acid residues.

150. A pharmaceutical composition comprising the block copolymer of claim 1 and a pharmaceutically acceptable excipient.

151. (canceled)

152. A method of treating or managing a condition of a subject comprising:
administering to the subject a therapeutically effective amount of the block copolymer of claim 1;
wherein the administering results in the treating or managing of a condition of the subject.

153. (canceled)

154. The method of claim 152, wherein the method results in an accumulation of P₁ at a disease site in the subject.

155. (canceled)

156. (canceled)

157. (canceled)

158. (canceled)

159. (canceled)

160. (canceled)

161. (canceled)

162. (canceled)

163. (canceled)

164. (canceled)

165. (canceled)

166. (canceled)

167. (canceled)

168. (canceled)


SEQUENCE LISTING (226 sequences)

SEQ ID NO: 1 - protein, 9 aa, synthetic construct
  REGION 1..9: note = Synthetic construct
LDEETGEFL

SEQ ID NO: 2 - protein, 9 aa, synthetic construct
  REGION 1..9: note = Synthetic construct
LDPETGEFL

SEQ ID NO: 3 - protein, 11 aa, synthetic construct
  REGION 1..11: note = Synthetic construct
LDEETGEFLRR

SEQ ID NO: 4 - protein, 12 aa, synthetic construct
  REGION 1..12: note = Synthetic construct
LDEETGEFLRRR

SEQ ID NO: 5 - protein, 13 aa, synthetic construct
  REGION 1..13: note = Synthetic construct
LDEETGEFLRRRR

SEQ ID NO: 6 - protein, 14 aa, synthetic construct
  REGION 1..14: note = Synthetic construct
LDEETGEFLRRRRR

SEQ ID NO: 7 - protein, 11 aa, synthetic construct
  REGION 1..11: note = Synthetic construct
RRLDEETGEFL

SEQ ID NO: 8 - protein, 12 aa, synthetic construct
  REGION 1..12: note = Synthetic construct
RRRLDEETGEFL

SEQ ID NO: 9 - protein, 13 aa, synthetic construct
  REGION 1..13: note = Synthetic construct
RRRRLDEETGEFL

SEQ ID NO: 10 - protein, 14 aa, synthetic construct
  REGION 1..14: note = Synthetic construct
RRRRRLDEETGEFL

SEQ ID NO: 11 - protein, 13 aa, synthetic construct
  REGION 1..13: note = Synthetic construct
RRLDEETGEFLRR

SEQ ID NO: 12 - protein, 15 aa, synthetic construct
  REGION 1..15: note = Synthetic construct
RRRLDEETGEFLRRR

SEQ ID NO: 13 - protein, 11 aa, synthetic construct
  REGION 1..11: note = Synthetic construct
RLDEETGEFLR

SEQ ID NO: 14 - protein, 12 aa, synthetic construct
  REGION 1..12: note = Synthetic construct
RLDEETGEFLRR

SEQ ID NO: 15 - protein, 12 aa, synthetic construct
  REGION 1..12: note = Synthetic construct
RRLDEETGEFLR

SEQ ID NO: 16 - protein, 13 aa, synthetic construct
  REGION 1..13: note = Synthetic construct
RLDEETGEFLRRR

SEQ ID NO: 17 - protein, 13 aa, synthetic construct
  REGION 1..13: note = Synthetic construct
RRRLDEETGEFLR

SEQ ID NO: 18 - protein, 14 aa, synthetic construct
  REGION 1..14: note = Synthetic construct
RLDEETGEFLRRRR

SEQ ID NO: 19 - protein, 14 aa, synthetic construct
  REGION 1..14: note = Synthetic construct
RRLDEETGEFLRRR

SEQ ID NO: 20 - protein, 14 aa, synthetic construct
  REGION 1..14: note = Synthetic sequence
RRRLDEETGEFLRR

SEQ ID NO: 21 - protein, 14 aa, synthetic construct
  REGION 1..14: note = Synthetic construct
RRRRLDEETGEFLR

SEQ ID NO: 22 - protein, 15 aa, synthetic construct
  REGION 1..15: note = Synthetic construct
RLDEETGEFLRRRRR

SEQ ID NO: 23 - protein, 15 aa, synthetic construct
  REGION 1..15: note = Synthetic construct
RRLDEETGEFLRRRR

SEQ ID NO: 24 - protein, 15 aa, synthetic construct
  REGION 1..15: note = Synthetic construct
RRRRLDEETGEFLRR

SEQ ID NO: 25 - protein, 15 aa, synthetic construct
  REGION 1..15: note = Synthetic construct
RRRRRLDEETGEFLR

SEQ ID NO: 26 - protein, 11 aa, synthetic construct
  REGION 1..11: note = Synthetic construct
LDEETGEFLKK

SEQ ID NO: 27 - protein, 12 aa, synthetic construct
  REGION 1..12: note = Synthetic construct
LDEETGEFLKKK

SEQ ID NO: 28 - protein, 13 aa, synthetic construct
  REGION 1..13: note = Synthetic construct
LDEETGEFLKKKK

SEQ ID NO: 29 - protein, 14 aa, synthetic construct
  REGION 1..14: note = Synthetic construct
LDEETGEFLKKKKK

SEQ ID NO: 30 - protein, 11 aa, synthetic construct
  REGION 1..11: note = Synthetic construct
KKLDEETGEFL

SEQ ID NO: 31 - protein, 12 aa, synthetic construct
  REGION 1..12: note = Synthetic construct
KKKLDEETGEFL

SEQ ID NO: 32 - protein, 13 aa, synthetic construct
  REGION 1..13: note = Synthetic construct
KKKKLDEETGEFL

SEQ ID NO: 33 - protein, 14 aa, synthetic construct
  REGION 1..14: note = Synthetic construct
KKKKKLDEETGEFL

SEQ ID NO: 34 - protein, 13 aa, synthetic construct
  REGION 1..13: note = Synthetic construct
KKLDEETGEFLKK

SEQ ID NO: 35 - protein, 15 aa, synthetic construct
  REGION 1..15: note = Synthetic construct
KKKLDEETGEFLKKK

SEQ ID NO: 36 - protein, 11 aa, synthetic construct
  REGION 1..11: note = Synthetic construct
KLDEETGEFLK

SEQ ID NO: 37 - protein, 12 aa, synthetic construct
  REGION 1..12: note = Synthetic construct
KLDEETGEFLKK

SEQ ID NO: 38 - protein, 12 aa, synthetic construct
  REGION 1..12: note = Synthetic construct
KKLDEETGEFLK

SEQ ID NO: 39 - protein, 13 aa, synthetic construct
  REGION 1..13: note = Synthetic construct
KLDEETGEFLKKK

SEQ ID NO: 40 - protein, 13 aa, synthetic construct
  REGION 1..13: note = Synthetic construct
KKKLDEETGEFLK

SEQ ID NO: 41 - protein, 14 aa, synthetic construct
  REGION 1..14: note = Synthetic construct
KLDEETGEFLKKKK

SEQ ID NO: 42 - protein, 14 aa, synthetic construct
  REGION 1..14: note = Synthetic construct
KKLDEETGEFLKKK

SEQ ID NO: 43 - protein, 14 aa, synthetic construct
  REGION 1..14: note = Synthetic construct
KKKLDEETGEFLKK

SEQ ID NO: 44 - protein, 14 aa, synthetic construct
  REGION 1..14: note = Synthetic construct
KKKKLDEETGEFLK

SEQ ID NO: 45 - protein, 15 aa, synthetic construct
  REGION 1..15: note = Synthetic construct
KLDEETGEFLKKKKK

SEQ ID NO: 46 - protein, 15 aa, synthetic construct
  REGION 1..15: note = Synthetic construct
KKLDEETGEFLKKKK

SEQ ID NO: 47 - protein, 15 aa, synthetic construct
  REGION 1..15: note = Synthetic construct
KKKKLDEETGEFLKK

SEQ ID NO: 48 - protein, 15 aa, synthetic construct
  REGION 1..15: note = Synthetic construct
KKKKKLDEETGEFLK

SEQ ID NO: 49 - protein, 13 aa, synthetic construct
  REGION 1..13: note = Synthetic construct
LDEETGEFLKRKR

SEQ ID NO: 50 - protein, 13 aa, synthetic construct
  REGION 1..13: note = Synthetic construct
KRKRLDEETGEFL

SEQ ID NO: 51 - protein, 13 aa, synthetic construct
  REGION 1..13: note = Synthetic construct
RKRKLDEETGEFL

SEQ ID NO: 52 - protein, 13 aa, synthetic construct
  REGION 1..13: note = Synthetic construct
LDEETGEFLRKRK

SEQ ID NO: 53 - protein, 13 aa, synthetic construct
  REGION 1..13: note = Synthetic construct
KKLDEETGEFLRR

SEQ ID NO: 54 - protein, 13 aa, synthetic construct
  REGION 1..13: note = Synthetic construct
RRLDEETGEFLKK

SEQ ID NO: 55 - protein, 13 aa, synthetic construct
  REGION 1..13: note = Synthetic construct
KLDEETGEFLRRR

SEQ ID NO: 56 - protein, 13 aa, synthetic construct
  REGION 1..13: note = Synthetic construct
KKKLDEETGEFLR

SEQ ID NO: 57 - protein, 13 aa, synthetic construct
  REGION 1..13: note = Synthetic construct
RRRLDEETGEFLK

SEQ ID NO: 58 - protein, 13 aa, synthetic construct
  REGION 1..13: note = Synthetic construct
KRLDEETGEFLKR

SEQ ID NO: 59 - protein, 13 aa, synthetic construct
  REGION 1..13: note = Synthetic construct
RKLDEETGEFLRK

SEQ ID NO: 60 - protein, 13 aa, synthetic construct
  REGION 1..13: note = Synthetic construct
RKLDEETGEFLKR

SEQ ID NO: 61 - protein, 13 aa, synthetic construct
  REGION 1..13: note = Synthetic construct
KRLDEETGEFLRK

SEQ ID NO: 62 - protein, 13 aa, synthetic construct
  REGION 1..13: note = Synthetic construct
LDEETGEFLKKRR

SEQ ID NO: 63 - protein, 13 aa, synthetic construct
  REGION 1..13: note = Synthetic construct
LDEETGEFLRRKK

SEQ ID NO: 64 - protein, 13 aa, synthetic construct
  REGION 1..13: note = Synthetic construct
KKRRLDEETGEFL

SEQ ID NO: 65 - protein, 13 aa, synthetic construct
  REGION 1..13: note = Synthetic construct
RRKKLDEETGEFL

SEQ ID NO: 66 - protein, 16 aa, synthetic construct
  REGION 1..16: note = Synthetic construct
LDEETGEFLGSGSGRR

SEQ ID NO: 67 - protein, 16 aa, synthetic construct
  REGION 1..16: note = Synthetic construct
GSGSGRRLDEETGEFL

SEQ ID NO: 68 - protein, 16 aa, synthetic construct
  REGION 1..16: note = Synthetic construct
LDEETGEFLGSGSGKK

SEQ ID NO: 69 - protein, 16 aa, synthetic construct
  REGION 1..16: note = Synthetic construct
GSGSGKKLDEETGEFL

SEQ ID NO: 70 - protein, 11 aa, synthetic construct
  REGION 1..11: note = Synthetic construct
LDPETGEFLRR

SEQ ID NO: 71 - protein, 12 aa, synthetic construct
  REGION 1..12: note = Synthetic construct
LDPETGEFLRRR

SEQ ID NO: 72 - protein, 13 aa, synthetic construct
  REGION 1..13: note = Synthetic construct
LDPETGEFLRRRR

SEQ ID NO: 73 - protein, 14 aa, synthetic construct
  REGION 1..14: note = Synthetic construct
LDPETGEFLRRRRR

SEQ ID NO: 74 - protein, 11 aa, synthetic construct
  REGION 1..11: note = Synthetic construct
RRLDPETGEFL

SEQ ID NO: 75 - protein, 12 aa, synthetic construct
  REGION 1..12: note = Synthetic construct
RRRLDPETGEFL

SEQ ID NO: 76 - protein, 13 aa, synthetic construct
  REGION 1..13: note = Synthetic construct
RRRRLDPETGEFL

SEQ ID NO: 77 - protein, 14 aa, synthetic construct
  REGION 1..14: note = Synthetic construct
RRRRRLDPETGEFL

SEQ ID NO: 78 - protein, 13 aa, synthetic construct
  REGION 1..13: note = Synthetic construct
RRLDPETGEFLRR

SEQ ID NO: 79 - protein, 15 aa, synthetic construct
  REGION 1..15: note = Synthetic construct
RRRLDPETGEFLRRR

SEQ ID NO: 80 - protein, 11 aa, synthetic construct
  REGION 1..11: note = Synthetic construct
RLDPETGEFLR

SEQ ID NO: 81 - protein, 12 aa, synthetic construct
  REGION 1..12: note = Synthetic construct
RLDPETGEFLRR

SEQ ID NO: 82 - protein, 12 aa, synthetic construct
  REGION 1..12: note = Synthetic construct
RRLDPETGEFLR

SEQ ID NO: 83 - protein, 13 aa, synthetic construct
  REGION 1..13: note = Synthetic construct
RLDPETGEFLRRR

SEQ ID NO: 84 - protein, 13 aa, synthetic construct
  REGION 1..13: note = Synthetic construct
RRRLDPETGEFLR

SEQ ID NO: 85 - protein, 14 aa, synthetic construct
  REGION 1..14: note = Synthetic construct
RLDPETGEFLRRRR

SEQ ID NO: 86 - protein, 14 aa, synthetic construct
  REGION 1..14: note = Synthetic construct
RRLDPETGEFLRRR

SEQ ID NO: 87 - protein, 14 aa, synthetic construct
  REGION 1..14: note = Synthetic construct
RRRLDPETGEFLRR

SEQ ID NO: 88 - protein, 14 aa, synthetic construct
  REGION 1..14: note = Synthetic construct
RRRRLDPETGEFLR

SEQ ID NO: 89 - protein, 15 aa, synthetic construct
  REGION 1..15: note = Synthetic construct
RLDPETGEFLRRRRR

SEQ ID NO: 90 - protein, 15 aa, synthetic construct
  REGION 1..15: note = Synthetic construct
RRLDPETGEFLRRRR

SEQ ID NO: 91 - protein, 15 aa, synthetic construct
  REGION 1..15: note = Synthetic construct
RRRRLDPETGEFLRR

SEQ ID NO: 92 - protein, 15 aa, synthetic construct
  REGION 1..15: note = Synthetic construct
RRRRRLDPETGEFLR

SEQ ID NO: 93 - protein, 11 aa, synthetic construct
  REGION 1..11: note = Synthetic construct
LDPETGEFLKK

SEQ ID NO: 94 - protein, 12 aa, synthetic construct
  REGION 1..12: note = Synthetic construct
LDPETGEFLKKK

SEQ ID NO: 95 - protein, 13 aa, synthetic construct
  REGION 1..13: note = Synthetic construct
LDPETGEFLKKKK

SEQ ID NO: 96 - protein, 14 aa, synthetic construct
  REGION 1..14: note = Synthetic construct
LDPETGEFLKKKKK

SEQ ID NO: 97 - protein, 11 aa, synthetic construct
  REGION 1..11: note = Synthetic construct
KKLDPETGEFL

SEQ ID NO: 98 - protein, 12 aa, synthetic construct
  REGION 1..12: note = Synthetic construct
KKKLDPETGEFL

SEQ ID NO: 99 - protein, 13 aa, synthetic construct
  REGION 1..13: note = Synthetic construct
KKKKLDPETGEFL

SEQ ID NO: 100 - protein, 14 aa, synthetic construct
  REGION 1..14: note = Synthetic construct
KKKKKLDPETGEFL

SEQ ID NO: 101 - protein, 13 aa, synthetic construct
  REGION 1..13: note = Synthetic construct
KKLDPETGEFLKK

SEQ ID NO: 102 - protein, 15 aa, synthetic construct
  REGION 1..15: note = Synthetic construct
KKKLDPETGEFLKKK

SEQ ID NO: 103 - protein, 11 aa, synthetic construct
  REGION 1..11: note = Synthetic construct
KLDPETGEFLK

SEQ ID NO: 104 - protein, 12 aa, synthetic construct
  REGION 1..12: note = Synthetic construct
KLDPETGEFLKK

SEQ ID NO: 105 - protein, 12 aa, synthetic construct
  REGION 1..12: note = Synthetic construct
KKLDPETGEFLK

SEQ ID NO: 106 - protein, 13 aa, synthetic construct
  REGION 1..13: note = Synthetic construct
KLDPETGEFLKKK

SEQ ID NO: 107 - protein, 13 aa, synthetic construct
  REGION 1..13: note = Synthetic construct
KKKLDPETGEFLK

SEQ ID NO: 108 - protein, 14 aa, synthetic construct
  REGION 1..14: note = Synthetic construct
KLDPETGEFLKKKK

SEQ ID NO: 109 - protein, 14 aa, synthetic construct
  REGION 1..14: note = Synthetic construct
KKLDPETGEFLKKK

SEQ ID NO: 110 - protein, 14 aa, synthetic construct
  REGION 1..14: note = Synthetic construct
KKKLDPETGEFLKK

SEQ ID NO: 111 - protein, 14 aa, synthetic construct
  REGION 1..14: note = Synthetic construct
KKKKLDPETGEFLK

SEQ ID NO: 112 - protein, 15 aa, synthetic construct
  REGION 1..15: note = Synthetic construct
KLDPETGEFLKKKKK

SEQ ID NO: 113 - protein, 15 aa, synthetic construct
  REGION 1..15: note = Synthetic construct
KKLDPETGEFLKKKK

SEQ ID NO: 114 - protein, 15 aa, synthetic construct
  REGION 1..15: note = Synthetic construct
KKKKLDPETGEFLKK

SEQ ID NO: 115 - protein, 15 aa, synthetic construct
  REGION 1..15: note = Synthetic construct
KKKKKLDPETGEFLK

SEQ ID NO: 116 - protein, 13 aa, synthetic construct
  REGION 1..13: note = Synthetic construct
LDPETGEFLKRKR

SEQ ID NO: 117 - protein, 13 aa, synthetic construct
  REGION 1..13: note = Synthetic construct
KRKRLDPETGEFL

SEQ ID NO: 118 - protein, 13 aa, synthetic construct
  REGION 1..13: note = Synthetic construct
RKRKLDPETGEFL

SEQ ID NO: 119 - protein, 13 aa, synthetic construct
  REGION 1..13: note = Synthetic construct
LDPETGEFLRKRK

SEQ ID NO: 120 - protein, 13 aa, synthetic construct
  REGION 1..13: note = Synthetic construct
KKLDPETGEFLRR

SEQ ID NO: 121 - protein, 13 aa, synthetic construct
  REGION 1..13: note = Synthetic construct
RRLDPETGEFLKK

SEQ ID NO: 122 - protein, 13 aa, synthetic construct
  REGION 1..13: note = Synthetic construct
KLDPETGEFLRRR

SEQ ID NO: 123 - protein, 13 aa, synthetic construct
  REGION 1..13: note = Synthetic construct
KKKLDPETGEFLR

SEQ ID NO: 124 - protein, 13 aa, synthetic construct
  REGION 1..13: note = Synthetic construct
RRRLDPETGEFLK

SEQ ID NO: 125 - protein, 13 aa, synthetic construct
  REGION 1..13: note = Synthetic construct
KRLDPETGEFLKR

SEQ ID NO: 126 - protein, 13 aa, synthetic construct
  REGION 1..13: note = Synthetic construct
RKLDPETGEFLRK

SEQ ID NO: 127 - protein, 13 aa, synthetic construct
  REGION 1..13: note = Synthetic construct
RKLDPETGEFLKR

SEQ ID NO: 128 - protein, 13 aa, synthetic construct
  REGION 1..13: note = Synthetic construct
KRLDPETGEFLRK

SEQ ID NO: 129 - protein, 13 aa, synthetic construct
  REGION 1..13: note = Synthetic construct
LDPETGEFLKKRR

SEQ ID NO: 130 - protein, 13 aa, synthetic construct
  REGION 1..13: note = Synthetic construct
LDPETGEFLRRKK

SEQ ID NO: 131 - protein, 13 aa, synthetic construct
  REGION 1..13: note = Synthetic construct
KKRRLDPETGEFL

SEQ ID NO: 132 - protein, 13 aa, synthetic construct
  REGION 1..13: note = Synthetic construct
RRKKLDPETGEFL

SEQ ID NO: 133 - protein, 16 aa, synthetic construct
  REGION 1..16: note = Synthetic construct
LDPETGEFLGSGSGRR

SEQ ID NO: 134 - protein, 16 aa, synthetic construct
  REGION 1..16: note = Synthetic construct
GSGSGRRLDPETGEFL

SEQ ID NO: 135 - protein, 16 aa, synthetic construct
  REGION 1..16: note = Synthetic construct
LDPETGEFLGSGSGKK

SEQ ID NO: 136 - protein, 16 aa, synthetic construct
  REGION 1..16: note = Synthetic construct
GSGSGKKLDPETGEFL

SEQ ID NO: 137 - protein, 8 aa, synthetic construct
  REGION 1..8: note = Synthetic construct
LDPTGEFL

SEQ ID NO: 138 - protein, 8 aa, synthetic construct
  REGION 1..8: note = Synthetic construct
LDPETGFL

SEQ ID NO: 139 - skipped

SEQ ID NO: 140 - protein, 16 aa, synthetic construct
  REGION 1..16: note = Synthetic construct
YGRKKRRLDPETGEFL

SEQ ID NO: 141 - protein, 16 aa, synthetic construct
  REGION 1..16: note = Synthetic construct
LDPETGEFLYGRKKRR

SEQ ID NO: 142 - protein, 8 aa, synthetic construct
AAKAAKAA

SEQ ID NO: 143 - protein, 5 aa, synthetic construct
AAKAA

SEQ ID NO: 144 - protein, 8 aa, synthetic construct
AAKAARAA

SEQ ID NO: 145 - protein, 6 aa, synthetic construct
AAKRAA

SEQ ID NO: 146 - protein, 6 aa, synthetic construct
AAKEAA

SEQ ID NO: 147 - protein, 6 aa, synthetic construct
AAEKAA

SEQ ID NO: 148 - protein, 8 aa, synthetic construct
AAEAAKAA

SEQ ID NO: 149 - protein, 6 aa, synthetic construct
AAKYAA

SEQ ID NO: 150 - protein, 11 aa, synthetic construct
GGGGEKGGGGG

SEQ ID NO: 151 - protein, 11 aa, synthetic construct
GGGGGKGGGGG

SEQ ID NO: 152 - protein, 11 aa, synthetic construct
GGGGGKEGGGG

SEQ ID NO: 153 - protein, 11 aa, synthetic construct
GGGGQKGGGGG

SEQ ID NO: 154 - protein, 15 aa, synthetic construct
GPLGLAGGWGERDGS

SEQ ID NO: 155 - protein, 15 aa, synthetic construct
GGSGSGSGWGERDGS

SEQ ID NO: 156 - protein, 10 aa, synthetic construct
ISYGNDALMP

SEQ ID NO: 157 - protein, 10 aa, synthetic construct
SLINGPAYMD

SEQ ID NO: 158 - protein, 11 aa, synthetic construct
GACLRSGRGCG

SEQ ID NO: 159 - protein, 11 aa, synthetic construct
GAALRSGRGAG

SEQ ID NO: 160 - protein, 6 aa, synthetic construct
PRCCGE

SEQ ID NO: 161 - protein, 36 aa, synthetic construct
MCMPCFTTDHQMARKCDDCCGGKGRGKCYGPQCLCR

SEQ ID NO: 162 - protein, 16 aa, synthetic construct
HWWQWPSSLQLRGGGS

SEQ ID NO: 163 - protein, 16 aa, synthetic construct
HNWTRWLLHPDRGGGS

SEQ ID NO: 164 - protein, 12 aa, synthetic construct
GACFSIAHECGA

SEQ ID NO: 165 - protein, 8 aa, synthetic construct
VMDGYPMP

SEQ ID NO: 166 - protein, 8 aa, synthetic construct
GYPKSALR

SEQ ID NO: 167 - protein, 15 aa, synthetic construct
NENLLRFFVAPFPEV

SEQ ID NO: 168 - protein, 10 aa, synthetic construct
CRVYGPYLLC

SEQ ID NO: 169 - protein, 18 aa, unidentified
ADGACGYGRFSPPCGAAG

SEQ ID NO: 170 - protein, 18 aa, synthetic construct
ADGACILWMDDGWCGAAG

SEQ ID NO: 171 - protein, 10 aa, synthetic construct
STTHWGFTLC

SEQ ID NO: 172 - protein, 28 aa, synthetic construct
SLRRSSCFGGRMDRIGAQSGLGCNSFRY

SEQ ID NO: 173 - protein, 28 aa, synthetic construct
SLRRSSCFGGRIDRIGAQSGLGCNSFRY

SEQ ID NO: 174 - protein, 35 aa, synthetic construct
MGSFSITLGFFLVLAFWLPGHIGPNPVYSAVSNTD

SEQ ID NO: 175 - protein, 45 aa, synthetic construct
SQGSTLRVQQRPQNSKVTHISSCFGHKIDRIGSVSRLGCNALKLL

SEQ ID NO: 176 - protein, 32 aa, synthetic construct
NSKMAHSSSCFGQKIDRIGAVSRLGCDGLRLF

SEQ ID NO: 177 - protein, 32 aa, synthetic construct
SPKMVQGSGCFGRKMDRISSSSGLGCKVLRRH

SEQ ID NO: 178 - protein, 22 aa, synthetic construct
GLSKGCFGLKLDRIGSMSGLGC

SEQ ID NO: 179 - protein, 38 aa, synthetic construct
EVKYDPCFGHKIDRINHVSNLGCPSLRDPRPNAPSTSA

SEQ ID NO: 180 - protein, 21 aa, synthetic construct
MAKSGIYLGCFILILIQNMVA

SEQ ID NO: 181 - protein, 25 aa, synthetic construct
AMVSEFLKQAWFIENEEQEYVQTVK

SEQ ID NO: 182 - protein, 32 aa, synthetic construct
TAPRSLRRSSCFGGRMDRIGAQSGLGCNSFRY

SEQ ID NO: 183 - protein, 37 aa, synthetic construct
DNKPPKKGPPNGCFGHKIDRIGSHSGLGCNKVDDNKG

SEQ ID NO: 184 - protein, 4 aa, synthetic construct
AKAK

SEQ ID NO: 185 - protein, 4 aa, synthetic construct
VKVK

SEQ ID NO: 186 - protein, 4 aa, synthetic construct
KKKK

SEQ ID NO: 187 - protein, 4 aa, synthetic construct
LKLK

SEQ ID NO: 188 - skipped

SEQ ID NO: 189 - protein, 11 aa, synthetic construct
GGGDQKGGGGG

SEQ ID NO: 190 - protein, 11 aa, synthetic construct
GGGDPKGGGGG

SEQ ID NO: 191 - protein, 11 aa, synthetic construct
GGGQEKGGGGG

SEQ ID NO: 192-194 - skipped (3 entries)

SEQ ID NO: 195 - protein, 12 aa, synthetic construct
ELSYGYDEKSTG

SEQ ID NO: 196 - protein, 7 aa, synthetic construct
GYDEKST

SEQ ID NO: 197 - protein, 7 aa, synthetic construct
GYDEKSA

SEQ ID NO: 198 - protein, 7 aa, synthetic construct
GFDEKAG

SEQ ID NO: 199 - protein, 7 aa, synthetic construct
GYDEKAG

SEQ ID NO: 200 - protein, 7 aa, synthetic construct
AYDVKSG

SEQ ID NO: 201 - protein, 7 aa, synthetic construct
SYDVKSG

SEQ ID NO: 202 - protein, 6 aa, synthetic construct
FDAKGG

SEQ ID NO: 203 - protein, 7 aa, synthetic construct
QYDGKGV

SEQ ID NO: 204 - protein, 7 aa, synthetic construct
QYDPSKA

SEQ ID NO: 205 - protein, 6 aa, synthetic construct
YSDKGV

SEQ ID NO: 206 - protein, 7 aa, synthetic construct
PQQEKAH

SEQ ID NO: 207 - protein, 7 aa, synthetic construct
GQREKGP

SEQ ID NO: 208 - protein, 7 aa, synthetic construct
IGAEKAG

SEQ ID NO: 209 - protein, 7 aa, synthetic construct
GAGEKGP

SEQ ID NO: 210 - protein, 7 aa, synthetic construct
VGGEKSG

SEQ ID NO: 211-212 - skipped (2 entries)

SEQ ID NO: 213 - protein, 4 aa, synthetic construct
AKGS

SEQ ID NO: 214 - protein, 9 aa, synthetic construct
CSTSMLKAC

SEQ ID NO: 215 - protein, 9 aa, synthetic construct
CKPGTSSYC

SEQ ID NO: 216 - protein, 9 aa, synthetic construct
CPDRSVNNC

SEQ ID NO: 217 - protein, 5 aa, synthetic construct
VPGGV

SEQ ID NO: 218 - protein, 5 aa, synthetic construct
KPGGV

SEQ ID NO: 219 - protein, 6 aa, synthetic construct
VPGVGV

SEQ ID NO: 220 - protein, 6 aa, synthetic construct
VPGKGV

SEQ ID NO: 221 - protein, 7 aa, synthetic construct
VAPGVGV

SEQ ID NO: 222 - protein, 7 aa, synthetic construct
VAPGKGV

SEQ ID NO: 223 - protein, 12 aa, synthetic construct
GYGSSSRRAPQT

SEQ ID NO: 224 - protein, 10 aa, synthetic construct
CTTHWGFTLC

SEQ ID NO: 225 - protein, 11 aa, synthetic construct
RPKPQQFFGLM

SEQ ID NO: 226 - protein, 201 aa, synthetic construct
VPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGVPGGKPGGKPGGKPGGKPGGKPGGKPGGKPGGKPGGKPGGKPGGV